(19) United States
(12) Patent Application Publication
(10) Pub. No.: US 2023/0270745 A1
(43) Pub. Date: Aug. 31, 2023

(54) METHODS OF TREATING HER2 POSITIVE CANCER WITH TUCATINIB IN COMBINATION WITH TRASTUZUMAB, A TAXANE, AND A VEGFR-2 ANTAGONIST

(71) Applicant: Seagen Inc., Bothell, WA (US)

(72) Inventor: Luke Walker, Bothell, WA (US)

(21) Appl. No.: 18/017,905

(22) PCT Filed: Jul. 28, 2021
(86) PCT No.: PCT/US2021/043395

§ 371 Date: Jan. 25, 2023

Related U.S. Application Data
(60) Provisional application No. 63/058,146, filed on Jul. 29, 2020.

(51) Int. Cl. A61K 31/517; A61K 39/395; A61K 31/337; A61P 35/00; A61P 11/00

(52) U.S. Cl. CPC A61K 31/517; A61K 39/3955; A61K 31/337; A61P 35/00; A61P 11/00

(57) ABSTRACT

The present disclosure relates to a method of treating a HER2 positive cancer in a subject in need thereof, the method comprising: administering to the subject a therapeutically effective amount of a combination therapy comprising tucatinib, trastuzumab, a taxane, and a VEGFR-2 antagonist.

### CLAIM OF PRIORITY

This application claims the benefit of U.S. Provisional Application Ser. No. 63/058,146, filed on Jul. 29, 2020. The entire contents of the foregoing are incorporated herein by reference.

### BACKGROUND

Encoded by the ERBB2 gene, human epidermal growth factor receptor 2 (HER2) is part of a family of 4 related receptor tyrosine kinases, which include HER1 (also known as epidermal growth factor receptor [EGFR]), HER2, HER3, and HER4. HER1-4 are single-pass transmembrane glycoprotein receptors containing an extracellular ligand binding region and an intracellular signaling domain. HER2 has no known ligand, but it is the preferred dimerization partner for the other HER family receptors. When overexpressed in tumors, HER2 forms ligand-independent homodimeric complexes that autophosphorylate. HER2 homo- or heterodimerization results in the activation of multiple signaling cascades, including the Ras/Raf/MEK/MAPK, PI3K/AKT, Src, and STAT pathways. These signaling pathways lead to cell proliferation, inhibition of apoptosis, and metastasis.

The treatment and prevention of HER2 positive cancers represents an unmet need. Cancers that are characterized by the overexpression of HER2 (referred to as HER2 positive cancers) are often correlated with poor prognosis or are resistant to many standard therapies. Accordingly, there is a need for new therapies that are effective for the treatment of cancers such as HER2 positive cancers or metastatic HER2 positive cancers.

All references cited herein, including patent applications, patent publications, and scientific literature, are herein incorporated by reference in their entirety, as if each individual reference were specifically and individually indicated to be incorporated by reference.

### SUMMARY

Provided herein are methods of treating cancer in a subject in need thereof, the method comprising administering to the subject a therapeutically effective amount of a combination therapy comprising tucatinib, trastuzumab, a taxane, and a vascular endothelial growth factor receptor 2 (VEGFR-2) antagonist.

Also provided herein are methods of treating cancer in a subject in need thereof, the method comprising: (a) identifying the subject as having a HER2 positive cancer; and (b) administering to the subject a therapeutically effective amount of a combination therapy comprising tucatinib, trastuzumab, a taxane, and a vascular endothelial growth factor receptor 2 (VEGFR-2) antagonist.

In some embodiments, the trastuzumab is administered to the subject at a dose of about 6 mg/kg. In some embodiments, the trastuzumab is administered to the subject at a dose of about 4 mg/kg. In some embodiments, the tucatinib is administered to the subject at a dose of about 150 mg to about 650 mg. In some embodiments, the tucatinib is administered twice daily. In some embodiments, the tucatinib is administered to the subject orally.

In some embodiments, the VEGFR-2 antagonist is selected from the group consisting of bevacizumab, ramucirumab, aflibercept, cetuximab, panitumumab, regorafenib, sunitinib, sorafenib, pazopanib, vandetanib, axitinib, cediranib, vatalanib, motesanib, lucatinib, intedanib, semaxanib, apatinib, lenvatinib, carbozantinib, and a combination thereof. In some embodiments, the VEGFR-2 antagonist is a monoclonal antibody selected from the group consisting of bevacizumab, ramucirumab, aflibercept, cetuximab, panitumumab, and combinations thereof. In some embodiments, the VEGFR-2 antagonist is ramucirumab. In some embodiments, the ramucirumab is administered to the subject at a dose of about 8 mg/kg.

In some embodiments, the taxane is selected from the group consisting of paclitaxel, docetaxel, cabazitaxel, larotaxel, BMS-184476, BMS-188797, BMS-275183, milataxel, ortaxel, TL-310, docosahexaenoic acid-paclitaxel (DHA-paclitaxel), nab paclitaxel, EndoTAG+paclitaxel, XRP9881, polymeric-micellar paclitaxel, RPR-109881A, a pharmaceutically acceptable salt or solvate thereof, and a combination thereof. In some embodiments, the taxane is selected from the group consisting of paclitaxel, docetaxel, and cabazitaxel. In some embodiments, the taxane is paclitaxel. In some embodiments, the paclitaxel is administered to the subject at a dose of about 50 mg/m² to about 100 mg/m². In some embodiments, the paclitaxel is administered to the subject at a dose of about 80 mg/m².

In some embodiments, the HER2 positive cancer is selected from the group consisting of gastric adenocarcinoma, gastroesophageal junction (GEC) adenocarcinoma, esophageal adenocarcinoma, colorectal carcinoma (CRC), cholangiocarcinoma, gallbladder carcinoma, gastric cancer, lung cancer, biliary cancers, bladder cancer, esophageal cancer, melanoma, ovarian cancer, liver cancer, prostate cancer, pancreatic cancer, small intestine cancer, non-small cell lung cancer, head and neck cancer, uterine cancer, cervical cancer, brain cancer, and breast cancer. In some embodiments, the HER2 positive cancer is selected from the group consisting of gastric adenocarcinoma, gastroesophageal junction (GEC) adenocarcinoma, esophageal adenocarcinoma, colorectal carcinoma (CRC), cholangiocarcinoma, gallbladder carcinoma, gastric cancer, lung cancer, biliary cancers, bladder cancer, esophageal cancer, melanoma, ovarian cancer, liver cancer, prostate cancer, pancreatic cancer, small intestine cancer, non-small cell lung cancer, head and neck cancer, uterine cancer, cervical cancer, and brain cancer. In some embodiments, the HER2 positive cancer is gastric adenocarcinoma. In some embodiments, the HER2 positive cancer is gastroesophageal junction (GEC) adenocarcinoma. In some embodiments, the HER2 positive cancer is unresectable or metastatic.

In some embodiments, the subject has been previously treated with a HER2-directed antibody. In some embodiments, the subject has not been previously treated with an anti-HER2 and/or an anti-EGFR tyrosine kinase inhibitor. In some embodiments, the subject has not been previously treated with a HER2-directed antibody-drug conjugate. In some embodiments, the wherein the anti-HER2/EGFR tyrosine kinase inhibitor is selected from the group consisting of tucatinib, lapatinib, neratinib, or afatinib. In some embodiments, the antibody-drug conjugate is selected from the group consisting of ado-trastuzumab (T-DM1) or trastuzumab deruxtecan (DS8201a).

In some embodiments, the subject has not been previously treated with an anthracycline. In some embodiments, the anthracycline is selected from the group consisting of doxorubicin, epirubicin, mitoxantrone, idarubicin, liposomal doxorubicin, and combinations thereof.

In some embodiments, the subject was previously treated with at least one anticancer therapy. In some embodiments, the at least one anticancer therapy is selected from the group consisting of trastuzumab, lapatinib, trastuzumab and a taxane, pertuzumab, and combinations thereof. In some embodiments, the subject is refractory to the at least one anticancer therapy. In some embodiments, the subject developed a brain metastasis during the previous treatment with the at least one anticancer therapy.

Provided herein are methods of treating a HER2 positive cancer in a subject that has exhibited an adverse event after starting treatment with a combination therapy comprising tucatinib, trastuzumab, a taxane, and a vascular endothelial growth factor receptor 2 (VEGFR-2) antagonist at an initial dosage level, comprising administering to the subject at least one component of the combination therapy at a reduced dosage level.

In some embodiments, the taxane is selected from the group consisting of paclitaxel, docetaxel, cabazitaxel, larotaxel, BMS-184476, BMS-188797, BMS-275183, milataxel, ortaxel, TL-310, docosahexaenoic acid-paclitaxel (DHA-paclitaxel), nab paclitaxel, EndoTAG+paclitaxel, XRP9881, polymeric-micellar paclitaxel, RPR-109881A, a pharmaceutically acceptable salt or solvate thereof, and a combination thereof. In some embodiments, the taxane is paclitaxel.

In some embodiments, paclitaxel is administered to the subject at an initial dose of about 50 mg/m² to about 100 mg/m². In some embodiments, the paclitaxel is administered to the subject at an initial dose of about 80 mg/m². In some embodiments, the paclitaxel is administered to the subject at a reduced dose of about 50 mg/m² to about 75 mg/m². In some embodiments, the paclitaxel is administered to the subject at a reduced dose of about 70 mg/m². In some embodiments, the paclitaxel is administered to the subject at a reduced dose of about 60 mg/m².

In some embodiments, the tucatinib is administered to the subject at an initial dose of about 150 mg to about 650 mg. In some embodiments, the tucatinib is administered to the subject at an initial dose of about 300 mg. In some embodiments, the tucatinib is administered to the subject at a reduced dose of about 125 mg to about 275 mg.

In some embodiments, the VEGFR-2 antagonist is selected from the group consisting of bevacizumab, ramucirumab, aflibercept, cetuximab, panitumumab, regorafenib, sunitinib, sorafenib, pazopanib, vandetanib, axitinib, cediranib, vatalanib, motesanib, lucatinib, intedanib, semaxanib, apatinib, lenvatinib, carbozantinib, and a combination thereof. In some embodiments, the VEGFR-2 antagonist is ramucirumab. In some embodiments, the ramucirumab is administered to the subject at an initial dose of about 8 mg/kg. In some embodiments, the ramucirumab is administered to the subject at a reduced dose of about 6 mg/kg. In some embodiments, the ramucirumab is administered to the subject at a reduced dose of about 5 mg/kg.

Also provided herein are methods of treating a HER2 positive cancer in a subject in need thereof, the method comprising: (a) administering to the subject a therapeutically effective amount of a combination therapy comprising tucatinib, trastuzumab, a taxane, and a vascular endothelial growth factor receptor 2 (VEGFR-2) antagonist; and (b) administering an effective amount of an anti-diarrheal agent.

Also provided herein are methods of reducing the severity or incidents of diarrhea, or preventing diarrhea in a subject having a HER2 positive cancer and being treated with an effective amount of a combination therapy comprising tucatinib, trastuzumab, a taxane, and a vascular endothelial growth factor receptor 2 (VEGFR-2) antagonist, the method comprising administering an effective amount of an anti-diarrheal agent prophylactically.

Also provided herein are methods of reducing the likelihood of a subject developing diarrhea, wherein the subject has a HER2 positive cancer and is being treated with an effective amount of a combination therapy comprising tucatinib, trastuzumab, a taxane, and a vascular endothelial growth factor receptor 2 (VEGFR-2) antagonist the method comprising administering an effective amount of an anti-diarrheal agent prophylactically.

In some embodiments, the combination therapy and the anti-diarrheal agent are administered concurrently. In some embodiments, the anti-diarrheal agent is administered prior to administration of the combination therapy. In some embodiments, the subject is exhibiting symptoms of diarrhea. In some embodiments, the subject is not exhibiting symptoms of diarrhea.

### DESCRIPTION OF THE DRAWINGS

FIG. 1 Shows a study schema including study visits, and the overall design of phase 2, and phase 3 associated with a phase 2/3 study of tucatinib in combination with trastuzumab, a taxane, and a VEGFR-2 antagonist for HER2+ positive cancers described herein in connection with Example 1.

FIG. 2 Shows a phase 2 study design associated with a phase 2/3 study of tucatinib in combination with trastuzumab, a taxane, and a VEGFR-2 antagonist for HER2+ positive cancers described herein in connection with Example 1.

FIG. 3 Shows a phase 3 study design associated with a phase 2/3 study of tucatinib in combination with trastuzumab, a taxane, and a VEGFR-2 antagonist for HER2+ positive cancers described herein in connection with Example 1.

FIG. 4 provides the amino acid sequence of the heavy (SEQ. ID NO. 1) and light chains (SEQ. ID NO. 2) of trastuzumab and the light chain variable domain (SEQ. ID NO. 3) and the heavy chain variable domain (SEQ. ID NO. 4).

FIGS. 5A-5C show that a combination of tucatinib and trastuzumab was active in HER2 amplified colorectal cancer (CRC) patient-derived xenograft (PDX) models. Data are shown as group mean+/−S.E.M. FIG. 5A shows the effects of tucatinib and trastuzumab, alone and in combination, on tumor growth in a CTG-0121 CRC PDX model. FIG. 5B shows the effects of tucatinib and trastuzumab, alone and in combination, on tumor growth in a CTG-0784 CRC PDX model. FIG. 5C shows the effects of tucatinib and trastuzumab, alone and in combination, on tumor growth in a CTG-0383 CRC PDX model.

FIGS. 6A and 6B show that a combination of tucatinib and trastuzumab was active in HER2 amplified esophageal cancer patient-derived xenograft (PDX) models. Data are shown as group mean+/−S.E.M. FIG. 6A shows the effects of tucatinib and trastuzumab, alone and in combination, on tumor growth in a CTG-0137 esophageal cancer PDX model. FIG. 6B shows the effects of tucatinib and trastuzumab, alone and in combination, on tumor growth in a CTG-0138 esophageal cancer PDX model.

FIGS. 7A-7C show that a combination of tucatinib and trastuzumab was active in HER2 positive gastric cancer patient-derived xenograft (PDX) models. Data are shown as group mean+/−S.D. FIG. 7A shows the effects of tucatinib and trastuzumab, alone and in combination, on tumor growth in a GXA 3038 gastric cancer PDX model. FIG. 7B shows the effects of tucatinib and trastuzumab, alone and in combination, on tumor growth in a GXA 3039 gastric cancer PDX model. FIG. 7C shows the effects of tucatinib and trastuzumab, alone and in combination, on tumor growth in a GXA 3054 gastric cancer PDX model.

FIG. 8 shows that a combination of tucatinib and trastuzumab was active in a CTG-0927 HER2 positive cholangiocarcinoma patient-derived xenograft (PDX) model. Data are shown as mean+/−S.E.M.

FIGS. 9A and 9B show that a combination of tucatinib and trastuzumab was active in HER2 positive non-small cell lung cancer (NSCLC) models. Data are shown as group mean+/−S.E.M. FIG. 9A shows the effects of tucatinib and trastuzumab, alone and in combination, on tumor growth in a Calu-3 NSCLC xenograft model. FIG. 9B shows the effects of tucatinib and trastuzumab, alone and in combination, on tumor growth in an NCI-H2170 NSCLC xenograft model.

### DETAILED DESCRIPTION

### I. Definitions

In order that the present disclosure can be more readily understood, certain terms are first defined. As used in this application, except as otherwise expressly provided herein, each of the following terms shall have the meaning set forth below. Additional definitions are set forth throughout the application.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure. For purposes of the present disclosure, the following terms are defined.

Units, prefixes, and symbols are denoted in their Système International de Unites (SI) accepted form. Numeric ranges are inclusive of the numbers defining the range. The headings provided herein are not limitations of the various aspects of the disclosure, which can be had by reference to the specification as a whole. Accordingly, the terms defined immediately below are more fully defined by reference to the specification in its entirety.

The terms “a,” “an,” or “the” as used herein not only include aspects with one member, but also include aspects with more than one member. For instance, the singular forms “a,” “an,” and “the” include plural referents unless the context clearly dictates otherwise. Thus, for example, reference to “a cell” includes a plurality of such cells and reference to “the agent” includes reference to one or more agents known to those skilled in the art, and so forth.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that aspects and embodiments of the disclosure described herein include “comprising,” “consisting,” and “consisting essentially of” aspects and embodiments. It is understood that aspects and variations of the embodiments described herein include “consisting of” and/or “consisting essentially of” aspects and variations. In some embodiments, methods consisting essentially of an administration step as disclosed herein include methods wherein a patient has failed a prior therapy (administered to the patient before the period of time) or has been refractory to such prior therapy, and/or wherein the cancer has metastasized or recurred. In some embodiments, methods consisting essentially of an administration step as disclosed herein include methods wherein a patient undergoes surgery, radiation, and/or other regimens prior to, substantially at the same time as, or following such an administration step as disclosed herein, and/or where the patient is administered other chemical and/or biological therapeutic agents following such an administration step as disclosed herein.

The terms “about” and “approximately” as used herein shall generally mean an acceptable degree of error for the quantity measured given the nature or precision of the measurements. Typical, exemplary degrees of error are within 20 percent (%), preferably within 10%, and more preferably within 5% of a given value or range of values. Any reference to “about X” specifically indicates at least the values X, 0.95X, 0.96X, 0.97X, 0.98X, 0.99X, 1.01X, 1.02X, 1.03X, 1.04X, and 1.05X. Thus, “about X” is intended to teach and provide written description support for a claim limitation of, e.g., “0.98X.” The terms “about” and “approximately,” particularly in reference to a given quantity, encompass and describe the given quantity itself.

Alternatively, in biological systems, the terms “about” and “approximately” may mean values that are within an order of magnitude, preferably within 5-fold, and more preferably within 2-fold of a given value. Numerical quantities given herein are approximate unless stated otherwise, meaning that the term “about” or “approximately” can be inferred when not expressly stated.

As used herein, the term “co-administering” includes sequential or simultaneous administration of tucatinib, trastuzumab, a taxane, and a vascular endothelial growth factor receptor 2 (VEGFR-2) antagonist (e.g., tucatinib, trastuzumab, paclitaxel, and ramucirumab). For example, the co-administered compounds can be administered by the same route. In other instances, the co-administered compounds are administered via different routes. For example, one or two compounds can be administered orally, and the other compound(s) can be administered, e.g., sequentially or simultaneously, via intravenous, intramuscular, subcutaneous, or intraperitoneal injection. The simultaneously or sequentially administered compounds or compositions can be administered such that tucatinib, trastuzumab, a taxane, and a VEGFR-2 antagonist (e.g., tucatinib, trastuzumab, paclitaxel, and ramucirumab) are simultaneously present in a subject or in a cell at an effective concentration.

As used herein, the term “combination”, “therapeutic combination”, “combination therapy”, or “pharmaceutical combination”, as used herein, defines either a fixed combination in one dosage unit form or a kit of parts or instructions for the combined administration where the tucatinib, trastuzumab, a taxane, and a VEGFR-2 antagonist (e.g., tucatinib, trastuzumab, paclitaxel, and ramucirumab) may be administered independently at the same time or separately within time intervals that allow that the combination partners show a cooperative, e.g., synergistic, effect.

A “cancer” refers to a broad group of various diseases characterized by the uncontrolled growth of abnormal cells in the body. A “cancer” or “cancer tissue” can include a tumor.

In the context of cancer, the term “stage” refers to a classification of the extent of cancer. Factors that are considered when staging a cancer include but are not limited to tumor size, tumor invasion of nearby tissues, and whether the tumor has metastasized to other sites. The specific criteria and parameters for differentiating one stage from another can vary depending on the type of cancer. Cancer staging can be used, for example, to assist in determining a prognosis or identifying the most appropriate treatment option(s).

One non-limiting example of a cancer staging system is referred to as the “TNM” system. In the TNM system, “T” refers to the size and extent of the main tumor, “N” refers to the number of nearby lymph nodes to which the cancer has spread, and “M” refers to whether the cancer has metastasized. “TX” denotes that the main tumor cannot be measured, “TO” denotes that the main tumor cannot be found, and “T1,” “T2,” “T3,” and “T4” denote the size or extent of the main tumor, wherein a larger number corresponds to a larger tumor or a tumor that has grown into nearby tissues. “NX” denotes that cancer in nearby lymph nodes cannot be measured, “NO” denotes that there is no cancer in nearby lymph nodes, and “N1,” “N2,” “N3,” and “N4” denote the number and location of lymph nodes to which the cancer has spread, wherein a larger number corresponds to a greater number of lymph nodes containing the cancer. “MX” denotes that metastasis cannot be measured, “M0” denotes that no metastasis has occurred, and “M1” denotes that the cancer has metastasized to other parts of the body.

As another non-limiting example of a cancer staging system, cancers are classified or graded as having one of five stages: “Stage 0,” “Stage I,” “Stage II,” “Stage III,” or “Stage IV.” Stage 0 denotes that abnormal cells are present, but have not spread to nearby tissue. This is also commonly called carcinoma in situ (CIS). CIS is not cancer, but may subsequently develop into cancer. Stages I, II, and III denote that cancer is present. Higher numbers correspond to larger tumor sizes or tumors that have spread to nearby tissues. Stage IV denotes that the cancer has metastasized. One of skill in the art will be familiar with the different cancer staging systems and readily be able to apply or interpret them.

The term “HER2” (also known as also known as HER2/neu, ERBB2, CD340, receptor tyrosine-protein kinase erbB-2, proto-oncogene Neu, and human epidermal growth factor receptor 2) refers to a member of the human epidermal growth factor receptor (HER/EGFR/ERBB) family of receptor tyrosine kinases. Amplification or overexpression of HER2 plays a significant role in the development and progression of certain aggressive types of cancer, including colorectal cancer, gastric cancer, gastric adenocarcinoma, gastroesophageal junction (GEJ) adenocarcinoma, lung cancer (e.g., non-small cell lung cancer (NSCLC)), biliary cancers (e.g., cholangiocarcinoma, gallbladder cancer), bladder cancer, esophageal cancer, melanoma, ovarian cancer, liver cancer, prostate cancer, pancreatic cancer, small intestine cancer, head and neck cancer, uterine cancer, cervical cancer, and breast cancer. Non-limiting examples of HER2 nucleotide sequences are set forth in GenBank reference numbers NP_001005862, NP_001289936, NP_001289937, NP_001289938, and NP_004448. Non-limiting examples of HER2 peptide sequences are set forth in GenBank reference numbers NP_001005862, NP_001276865, NP_001276866, NP_001276867, and NP_004439. Each of these sequences are hereby incorporated by reference in their entireties.

When HER2 is amplified or overexpressed in or on a cell, the cell is referred to as being “HER2 positive.” The level of HER2 amplification or overexpression in HER2 positive cells is commonly expressed as a score ranging from 0 to 3 (i.e., HER2 0, HER2 1+, HER2 2+, or HER2 3+), with higher scores corresponding to greater degrees of expression.

The term “HER2 positive-associated” with respect to a disease or disorder, as used herein refers to diseases or disorders associated with amplification or overexpression of HER2. Non-limiting examples of HER2 positive-associated diseases or disorders can include, for example, HER2 positive breast cancer (e.g., “HER2 positive breast cancer-associated”).

The term “metastasis” is an art known term that refers to the spread of cancer cells from the place where they first formed (the primary site) to one or more other sites in a subject (one or more secondary sites). In metastasis, cancer cells break away from the original (primary) tumor, travel through the blood or lymph system, and form a new tumor (a metastatic tumor) in other organs or tissues of the body. The new, metastatic tumor includes the same or similar cancer cells as the primary tumor. At the secondary site, the tumor cell may proliferate and begin the growth or colonization of a secondary tumor at this distant site.

The term “metastatic cancer” (also known as “secondary cancer”) as used herein refers to a type of cancer that originates in one tissue type, but then spreads to one or more tissues outside of the (primary) cancer's origin. Following metastasis, the distal tumors can be said to be “derived from” the pre-metastasis tumor. For example, a “tumor derived from” a breast cancer refers to a tumor that can be the result of a metastasized breast cancer. Metastatic brain cancer refers to cancer in the brain, i.e., cancer which originated in a tissue other than the brain and has metastasized to the brain.

The term “tucatinib,” also known as ONT-380, ARRY-380, and TUKYSA™, refers to the small molecule tyrosine kinase inhibitor that suppresses or blocks HER2 activation. Tucatinib has the following structure:

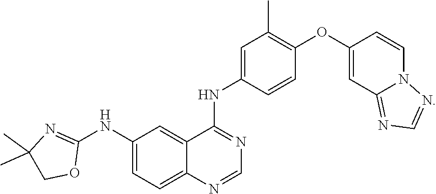 [SMILES: Cc1cc(Nc2ncnc3ccc(NC4=NC(C)(C)CO4)cc23)ccc1Oc1ccn2ncnc2c1]

In some instances, tucatinib can be in the form of a pharmaceutically acceptable salt.

Non-limiting examples of a taxane include paclitaxel, docetaxel, cabazitaxel, larotaxel, BMS-184476, BMS-188797, BMS-275183, milataxel, ortaxel, TL-310, docosahexaenoic acid-paclitaxel (DHA-paclitaxel), nab paclitaxel, EndoTAG+paclitaxel, XRP9881, polymeric-micellar paclitaxel, RPR-109881A, a pharmaceutically acceptable salt or solvate thereof, and a combination thereof.

The term “vascular endothelial growth factor receptor 2 (VEGFR-2) antagonist”, as used herein refers to a receptor ligand or drug that can reduce, decrease, or otherwise mitigate a biological response by binding and/or blocking a vascular endothelial growth factor receptor 2. Non-limiting examples of a VEGFR-2 antagonist include: bevacizumab, ramucirumab, aflibercept, cetuximab, panitumumab, regorafenib, sunitinib, sorafenib, pazopanib, vandetanib, axitinib, cediranib, vatalanib, and motesanib.

The term “anti-HER2 antibody-drug conjugate” refers to an anti-HER2 antibody conjugated to a therapeutic agent (i.e., a drug) optionally via a linker.

An “anti-HER2 antibody”, as used herein, refers to an antibody that binds to the HER2 protein. Anti-HER2 antibodies used for the treatment of cancer are typically monoclonal, although polyclonal antibodies are not excluded by the term. Anti-HER2 antibodies inhibit HER2 activation or downstream signaling by various mechanisms. As non-limiting examples, anti-HER2 antibodies can prevent ligand binding, receptor activation or receptor signal propagation, result in reduced HER2 expression or localization to the cell surface, inhibit HER2 cleavage, or induce antibody-mediated cytotoxicity. Non-limiting examples of anti-HER2 antibodies that are suitable for use in the methods and compositions of the present invention include trastuzumab, pertuzumab, margetuximab, and combinations thereof.

The term “ado-trastuzumab emtansine”, also known as T-DM1, refers to an antibody-drug conjugate composed of trastuzumab, a thioether linker, and a derivative of the antimitotic agent maytansine (also known as DM1). Ado-trastuzumab emtansine is sold in the U.S. under the trade name KADCYCLA®. As used herein, “ado-trastuzumab emtansine” also includes biosimilars of trastuzumab, for example, Kanjinti (trastuzumab-anns).

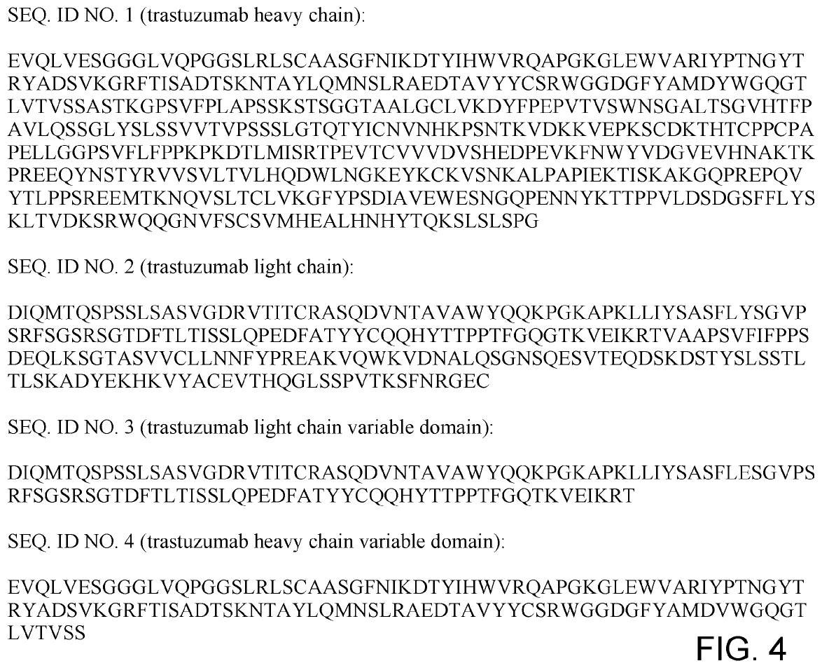

A “biosimilar” as used herein refers to an antibody or antigen-binding fragment that has the same primary amino acid sequence as compared to a reference antibody (e.g., trastuzumab) and optionally, may have detectable differences in post-translation modifications (e.g., glycosylation and/or phosphorylation) as compared to the reference antibody (e.g., a different glycoform). As a reference, the amino acid sequence of the heavy chain of trastuzumab is provided as SEQ. ID NO. 1, the light chain of trastuzumab is provided as SEQ. ID NO. 2, the light chain variable domain (SEQ. ID NO. 3), and the heavy chain variable domain (SEQ. ID NO. 4) (see also FIG. 4 and U.S. Pat. No. 5,821,337, which is incorporated herein in its entirety).

In some embodiments, a biosimilar is an antibody or antigen-binding fragment thereof that has a light chain that has the same primary amino acid sequence as compared to a reference antibody (e.g., trastuzumab) and a heavy chain that has the same primary amino acid sequence as compared to the reference antibody. In some examples, a biosimilar is an antibody or antigen-binding fragment thereof that has a light chain that includes the same light chain variable domain sequence as a reference antibody (e.g., trastuzumab) and a heavy chain that includes the same heavy chain variable domain sequence as a reference antibody. In some embodiments, a biosimilar can have a similar glycosylation pattern as compared to the reference antibody (e.g., trastuzumab). In other embodiments, a biosimilar can have a different glycosylation pattern as compared to the reference antibody (e.g., trastuzumab).

The term “tumor growth inhibition (TGI) index” refers to a value used to represent the degree to which an agent (e.g., tucatinib, trastuzumab, a taxane, and a VEGFR-2 antagonist or a combination thereof) inhibits the growth of a tumor when compared to an untreated control. The TGI index is calculated for a particular time point (e.g., a specific number of days into an experiment or clinical trial) according to the following formula:

$$TGI = 1 - ( \frac{{Volume}_{treated ( Tx Day X )} - {Volume}_{t reated ( Tx Day 0 )}}{{Volume}_{control ( Tx Day X )} - {Volume}_{c ontrol ( Tx Day 0 )}} ) \times 100 \% ,$$

where “Tx Day 0” denotes the first day that treatment is administered (i.e., the first day that an experimental therapy or a control therapy (e.g., vehicle only) is administered) and “Tx Day X” denotes X number of days after Day 0. Typically, mean volumes for treated and control groups are used. As a non-limiting example, in an experiment where study day 0 corresponds to “Tx Day 0” and the TGI index is calculated on study day 28 (i.e., “Tx Day 28”), if the mean tumor volume in both groups on study day 0 is 250 mm³ and the mean tumor volumes in the experimental and control groups are 125 mm³ and 750 mm³, respectively, then the TGI index on day 28 is 125%.

As used herein, the term “synergistic” or “synergy” refers to a result that is observed when administering a combination of components or agents (e.g., a combination of tucatinib, trastuzumab, a taxane, and a VEGFR-2 antagonist) produces an effect (e.g., inhibition of tumor growth, prolongation of survival time) that is greater than the effect that would be expected based on the additive properties or effects of the individual components. In some embodiments, synergism is determined by performing a Bliss analysis (see, e.g., Foucquier et al. Pharmacol. Res. Perspect. (2015) 3(3):e00149; hereby incorporated by reference in its entirety for all purposes). The Bliss Independence model assumes that drug effects are outcomes of probabilistic processes, and assumes that the drugs act completely independently (i.e., the drugs do not interfere with one another (e.g., the drugs have different sites of action) but each contributes to a common result).

The observed effect of a combination of drugs can be based on, for example, the TGI index, tumor size (e.g., volume, mass), an absolute change in tumor size (e.g., volume, mass) between two or more time points (e.g., between the first day a treatment is adminstered and a particular number of days after treatment is first administered), the rate of change of tumor size (e.g., volume, mass) between two or more time points (e.g., between the first day a treatment is adminstered and a particular number of days after treatment is first administered), or the survival time of a subject or a population of subjects. When the TGI index is taken as a measure of the observed effect of a combination of drugs, the TGI index can be determined at one or more time points. When the TGI index is determined at two or more time points, in some instances the mean or median value of the multiple TGI indices can be used as a measure of the observed effect. Furthermore, the TGI index can be determined in a single subject or a population of subjects. When the TGI index is determined in a population, the mean or median TGI index in the population (e.g., at one or more time points) can be used as a measure of the observed effect. When tumor size or the rate of tumor growth is used as a measure of the observed effect, the tumor size or rate of tumor growth can be measured in a subject or a population of subjects. In some instances, the mean or median tumor size or rate of tumor growth is determined for a subject at two or more time points, or among a population of subjects at one or more time points. When survival time is measured in a population, the mean or median survival time can be used as a measure of the observed effect.

When TGI indices are taken as a measure of the observed effects, the TGI indices can be determined at one or more time points. When TGI indices are determined at two or more time points, in some instances the mean or median values can be used as measures of the observed effects. Furthermore, the TGI indices can be determined in a single subject or a population of subjects in each treatment group. When the TGI indices are determined in populations of subjects, the mean or median TGI indices in each population (e.g., at one or more time points) can be used as measures of the observed effects. When tumor sizes or the rates of tumor growth are used as measures of the observed effects, the tumor sizes or rates of tumor growth can be measured in a subject or a population of subjects in each treatment group. In some instances, the mean or median tumor sizes or rates of tumor growth are determined for subjects at two or more time points, or among populations of subjects at one or more time points. When survival time is measured in a population, mean or median survival times can be used as measures of the observed effects.

In some embodiments, a combination of tucatinib, trastuzumab, a taxane, and a VEGFR-2 antagonist is considered to be synergistic when the combination produces an observed TGI index that is greater than the predicted TGI index for the combination of drugs (e.g., when the predicted TGI index is based upon the assumption that the drugs produced a combined effect that is additive). In some instances, the combination is considered to be synergistic when the observed TGI index is at least about 1%, 2%, 3%, 4%, 5%, 6%, 7%, 8%, 9%, 10%, 11%, 12, 13%, 14%, 15%, 16%, 17%, 18%, 19%, 20%, 25%, 30%, 35%, 40%, 45%, 50%, 55%, 60%, 65%, 70%, 75%, or 80% greater than the predicted TGI index for the combination of drugs.

In some embodiments, the rate of tumor growth (e.g., the rate of change of the size (e.g., volume, mass) of the tumor) is used to determine whether a combination of drugs is synergistic (e.g., the combination of drugs is synergistic when the rate of tumor growth is slower than would be expected if the combination of drugs produced an additive effect). In other embodiments, survival time is used to determine whether a combination of drugs is synergistic (e.g., a combination of drugs is synergistic when the survival time of a subject or population of subjects is longer than would be expected if the combination of drugs produced an additive effect).

“Treatment” or “therapy” of a subject refers to any type of intervention or process performed on, or the administration of an active agent to, the subject with the objective of reversing, alleviating, ameliorating, inhibiting, slowing down, or preventing the onset, progression, development, severity, or recurrence of a symptom, complication, condition, or biochemical indicia associated with a disease. In some embodiments, the disease is cancer. As used herein, the terms “treatment” and “treating” when referring, e.g., to the treatment of a cancer, are not intended to be absolute terms. For example, “treatment of cancer” and “treating cancer”, as used in a clinical setting, is intended to include obtaining beneficial or desired clinical results and can include an improvement in the condition of a subject having cancer. Beneficial or desired clinical results include, but are not limited to, one or more of the following: reducing the proliferation of (or destroying) neoplastic or cancerous cells, inhibiting metastasis of neoplastic cells, a decrease in metastasis in a subject, shrinking or decreasing the size of a tumor, change in the growth rate of one or more tumor(s) in a subject, an increase in the period of remission for a subject (e.g., as compared to the one or more metric(s) in a subject having a similar cancer receiving no treatment or a different treatment, or as compared to the one or more metric(s) in the same subject prior to treatment), decreasing symptoms resulting from a disease, increasing the quality of life of those suffering from a disease (e.g., assessed using FACT-G or EORTC-QLQC30), decreasing the dose of other medications required to treat a disease, delaying the progression of a disease, and/or prolonging survival of subjects having a disease.

The term “prophylactic” or “prophylactically” refers to any type of intervention or process performed on, or the administration of an active agent to, the subject with the objective of protecting or preventing a disease or condition from developing or at least not developing fully (e.g., to reduce the symptoms or severity of the disease or condition) such as in the development of a side effect (e.g., diarrhea).

A “subject” includes any human or non-human animal. The term “non-human animal” includes, but is not limited to, vertebrates such as non-human primates, sheep, dogs, and rodents such as mice, rats, and guinea pigs. In some embodiments, the subject is a human. The terms “subject” and “patient” and “individual” are used interchangeably herein.

An “effective amount” or “therapeutically effective amount” or “therapeutically effective dosage” of a drug or therapeutic agent is any amount of the drug that, when used alone or in combination with another therapeutic agent, protects a subject against the onset of a disease or promotes disease regression evidenced by a decrease in severity of disease symptoms, an increase in frequency and duration of disease symptom-free periods, or a prevention of impairment or disability due to the disease affliction. The ability of a therapeutic agent to promote disease regression can be evaluated using a variety of methods known to the skilled practitioner, such as in human subjects during clinical trials, in animal model systems predictive of efficacy in humans, or by assaying the activity of the agent in in vitro assays.

By way of example for the treatment of tumors, a therapeutically effective amount of an anti-cancer agent inhibits cell growth or tumor growth by at least about 10%, by at least about 20%, by at least about 30%, by at least about 40%, by at least about 50%, by at least about 60%, by at least about 70%, or by at least about 80%, by at least about 90%, by at least about 95%, by at least about 96%, by at least about 97%, by at least about 98%, or by at least about 99% in a treated subject(s) (e.g., one or more treated subjects) relative to an untreated subject(s) (e.g., one or more untreated subjects). In some embodiments, a therapeutically effective amount of an anti-cancer agent inhibits cell growth or tumor growth by 100% in a treated subject(s) (e.g., one or more treated subjects) relative to an untreated subject(s) (e.g., one or more untreated subjects).

In other embodiments of the disclosure, tumor regression (e.g., brain metastasis regression) can be observed and continue for a period of at least about 20 days, at least about 30 days, at least about 40 days, at least about 50 days, or at least about 60 days.

As used herein, “subtherapeutic dose” means a dose of a therapeutic compound (e.g., tucatinib) that is lower than the usual or typical dose of the therapeutic compound when administered alone for the treatment of a hyperproliferative disease (e.g., cancer).

“Simultaneous administration,” as used herein, means that the two or more therapies (e.g., in a combination therapy) are administered with a time separation of no more than about 15 minutes, such as no more than about any of 10, 5, or 1 minutes. When the two or more therapies are administered simultaneously, the two or more therapies can be contained in the same composition (e.g., a composition comprising both a first and second therapy) or in separate compositions (e.g., a first therapy in one composition and a second therapy is contained in another composition).

As used herein, the term “sequential administration” means that the two or more therapies (e.g., in a combination therapy) are administered with a time separation of more than about 15 minutes, such as more than about any of 20, 30, 40, 50, 60, or more minutes. Any of the two or more therapies may be administered first. The two or more therapies are contained in separate compositions, which may be contained in the same or different packages or kits.

As used herein, the term “concurrent administration” means that the administration of two or more therapies (e.g., in a combination therapy) overlap with each other. For example, the two or more therapies can be administered in the same day, or with a time separation of within one day, within two days, within three days, within four days, within five days, within six days, within seven days, within ten days, within fourteen days, or within twenty-one days.

By way of example, an “anti-cancer agent” promotes cancer regression in a subject. In some embodiments, a therapeutically effective amount of the drug promotes cancer regression to the point of eliminating the cancer. “Promoting cancer regression” means that administering an effective amount of the drug, alone or in combination with an anti-cancer agent, results in a reduction in tumor growth or size, necrosis of the tumor, a decrease in severity of at least one disease symptom, an increase in frequency and duration of disease symptom-free periods, or a prevention of impairment or disability due to the disease affliction. In addition, the terms “effective” and “effectiveness” with regard to a treatment includes both pharmacological effectiveness and physiological safety. Pharmacological effectiveness refers to the ability of the drug to promote cancer regression in the patient. Physiological safety refers to the level of toxicity or other adverse physiological effects at the cellular, organ and/or organism level (adverse effects) resulting from administration of the drug.

“Sustained response” refers to the sustained effect on reducing tumor growth after cessation of a treatment. For example, the tumor size can remain to be the same or smaller as compared to the size at the beginning of the administration phase. In some embodiments, the sustained response has a duration that is at least the same as the treatment duration, or at least 1.5, 2.0, 2.5, or 3 times longer than the treatment duration.

As used herein, “complete response” or “CR” refers to disappearance of all target lesions; “partial response” or “PR” refers to at least a 30% decrease in the sum of the longest diameters (SLD) of target lesions, taking as reference the baseline SLD; and “stable disease” or “SD” refers to neither sufficient shrinkage of target lesions to qualify for PR, nor sufficient increase to qualify for PD, taking as reference the smallest SLD since the treatment started.

As used herein, “progression free survival” or “PFS” refers to the length of time during and after treatment during which the disease being treated (e.g., breast cancer) does not get worse. Progression-free survival may include the amount of time patients have experienced a complete response or a partial response, as well as the amount of time patients have experienced stable disease.

As used herein, “overall response rate” or “ORR” refers to the sum of complete response (CR) rate and partial response (PR) rate.

As used herein, “overall survival” or “OS” refers to the percentage of individuals in a group who are likely to be alive after a particular duration of time.

The term “weight-based dose”, as referred to herein, means that a dose administered to a subject is calculated based on the weight of the subject. For example, when a subject with 60 kg body weight requires 3.6 mg/kg of an agent, such as tucatinib, trastuzumab, a taxane, and a VEGFR-2 antagonist one can calculate and use the appropriate amount of the agent (i.e., 216 mg) for administration to said subject.

The use of the term “fixed dose” with regard to a method of the disclosure means that two or more different agents (e.g., tucatinib, trastuzumab, a taxane, and a VEGFR-2 antagonist) are administered to a subject in particular (fixed) ratios with each other. In some embodiments, the fixed dose is based on the amount (e.g., mg) of the agents. In certain embodiments, the fixed dose is based on the concentration (e.g., mg/ml) of the agents.

The use of the term “flat dose” with regard to the methods and dosages of the disclosure means a dose that is administered to a subject without regard for the weight or body surface area (BSA) of the subject. The flat dose is therefore not provided as a mg/kg dose, but rather as an absolute amount of the agent (e.g., tucatinib, trastuzumab, a taxane, and a VEGFR-2 antagonist). For example, a subject with 60 kg body weight and a subject with 100 kg body weight can receive the same dose of tucatinib (e.g., 300 mg).

The phrase “pharmaceutically acceptable” indicates that the substance or composition must be compatible chemically and/or toxicologically, with the other ingredients comprising a formulation, and/or the mammal being treated therewith.

As used herein, the term “pharmaceutically acceptable carrier” refers to a substance that aids the administration of an active agent to a cell, an organism, or a subject. “Pharmaceutically acceptable carrier” refers to a carrier or excipient that can be included in the compositions of the disclosure and that causes no significant adverse toxicological effect on the subject. Non-limiting examples of pharmaceutically acceptable carriers include water, NaCl, normal saline solutions, lactated Ringer's, normal sucrose, normal glucose, binders, fillers, disintegrants, lubricants, coatings, sweeteners, flavors and colors, liposomes, dispersion media, microcapsules, cationic lipid carriers, isotonic and absorption delaying agents, and the like. The carrier may also be substances for providing the formulation with stability, sterility and isotonicity (e.g., antimicrobial preservatives, antioxidants, chelating agents and buffers), for preventing the action of microorganisms (e.g. antimicrobial and antifungal agents, such as parabens, chlorobutanol, phenol, sorbic acid and the like) or for providing the formulation with an edible flavor etc. In some instances, the carrier is an agent that facilitates the delivery of a small molecule drug or antibody to a target cell or tissue. One of skill in the art will recognize that other pharmaceutical carriers are useful in the present disclosure.

The phrase “pharmaceutically acceptable salt” as used herein, refers to pharmaceutically acceptable organic or inorganic salts of a compound of the disclosure. Exemplary salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate (mesylate), ethanesulfonate, benzenesulfonate, p-toluenesulfonate, pamoate (i.e., 4,4′-methylene-bis-(2-hydroxy-3-naphthoate)) salts, alkali metal (e.g., sodium and potassium) salts, alkaline earth metal (e.g., magnesium) salts, and ammonium salts. A pharmaceutically acceptable salt may involve the inclusion of another molecule such as an acetate ion, a succinate ion or other counter ion. The counter ion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counter ion.

As used herein, the term “solid dispersion” means a system in a solid state comprising at least two components, wherein one component is dispersed throughout the other component. For example, a solid dispersion as described herein can include one component of tucatinib dispersed throughout another component, such as a dispersion polymer.

As used herein, the term “amorphous” means a solid in a solid state that is a non-crystalline state. Amorphous solids generally possess crystal-like short range molecular arrangement, but no long range order of molecular packing as found in crystalline solids. The solid state form of a solid may be determined by polarized light microscopy, X-ray powder diffraction (“XRPD”), differential scanning calorimetry (“DSC”), or other standard techniques known to those of skill in the art.

As used herein, the term “amorphous solid dispersion” means a solid comprising a drug substance and a dispersion polymer. The amorphous solid dispersion discussed herein comprises amorphous tucatinib and a dispersion polymer, wherein the amorphous solid dispersion contains tucatinib in a substantially amorphous solid state form. In certain embodiments, the substantially amorphous solid state form means that the tucatinib component in the amorphous solid dispersion is at least 80% amorphous tucatinib. In certain embodiments, the substantially amorphous solid state form means that the tucatinib component in the amorphous solid dispersion is at least 85% amorphous tucatinib. In certain embodiments, the substantially amorphous solid state form means that the tucatinib component in the amorphous solid dispersion is at least 90% tucatinib. In certain embodiments, the substantially amorphous solid state form means that the tucatinib component in the amorphous solid dispersion is at least 95% amorphous tucatinib.

As used herein, the term “dispersion polymer” means a polymer that allows for tucatinib to be dispersed throughout such that a solid dispersion may form. The dispersion polymer is preferably neutral or basic. The dispersion polymer may contain a mixture of two or more polymers. Examples of dispersion polymers include, but are not limited to, vinyl polymers and copolymers, vinylpyrrolidine vinylacetate copolymer (“PVP-VA”), polyvinyl alcohols, polyvinyl alcohol polyvinyl acetate copolymers, polyvinyl pyrrolidine (“PVP”), acrylate and methacrylate copolymers, methylacrylic acid methyl methacrylate copolymer (such as Eudragit®), polyethylene polyvinyl alcohol copolymers, polyoxyethylene-polyoxypropylene block copolymers (also referred to as poloxamers), graft copolymer comprised of polyethylene glycol, polyvinyl caprolactam and polyvinyl acetate (such as Soluplus®), cellulosic polymers, such as hydroxypropyl methyl cellulose acetate (“HPMCA”), hydroxypropyl methyl cellulose (“HPMC”), hydroxypropyl cellulose (“HPC”), methyl cellulose, hydroxyethyl methyl cellulose, hydroxyethyl cellulose, hydroxyethyl cellulose acetate, and hydroxyethyl ethyl cellulose, hydroxypropyl methyl cellulose acetate succinate (“HPMCAS”), hydroxypropyl methyl cellulose phthalate (“HPMCP”), carboxymethylethyl cellulose (“CMEC”), cellulose acetate phthalate (“CAP”), cellulose acetate succinate (“CAS”), hydroxypropyl methyl cellulose acetate phthalate (“HPMCAP”), cellulose acetate trimellitate (“CAT”), hydroxypropyl methyl cellulose acetate trimellitate (“HPMCAT”), and carboxymethylcellulose acetate butyrate (“CMCAB”), and the like.

As used herein, the term “spray drying” means processes involved in breaking up liquid mixtures into small droplets (atomization) and rapidly removing solvent from the mixture in a spray drying apparatus where there is a strong driving force for evaporation of solvent from the droplets. The phrase spray drying is used conventionally and broadly. Spray drying processes and spray drying equipment are described generally in Perry, Robert H., and Don W. Green (eds.). Perry's Chemical Engineers' Handbook. New York: McGraw-Hill, 2007 (8^{th} edition).

As used herein, “polymorphs” refer to distinct solids sharing the same molecular formula, yet each polymorph may have distinct solid state physical properties. A single compound may give rise to a variety of polymorphic forms where each form has different and distinct solid state physical properties, such as different solubility profiles, melting point temperatures, flowability, dissolution rates and/or different X-ray diffraction peaks. These practical physical characteristics are influenced by the conformation and orientation of molecules in the unit cell, which defines a particular polymorphic form of a substance. Polymorphic forms of a compound can be distinguished in a laboratory by X-ray diffraction spectroscopy, such as X-ray powder diffraction (“XRPD”), and by other methods, such as infrared spectrometry. Additionally, polymorphic forms of the same drug substance or active pharmaceutical ingredient can be administered by itself or formulated as a drug product (pharmaceutical composition) and are well known in the pharmaceutical art to affect, for example, the solubility, stability, flowability, tractability and compressibility of drug substances and the safety and efficacy of drug products. For more, see Hilfiker, Rolf (ed.), Polymorphism in the Pharmaceutical Industry. Weinheim, Germany: Wiley-VCH 2006.

“Administering” or “administration” refer to the physical introduction of a therapeutic agent to a subject, using any of the various methods and delivery systems known to those skilled in the art. Exemplary routes of administration include oral, intravenous, intramuscular, subcutaneous, intraperitoneal, spinal or other parenteral routes of administration, for example by injection or infusion (e.g., intravenous infusion). The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. A therapeutic agent can be administered via a non-parenteral route, or orally. Other non-parenteral routes include a topical, epidermal or mucosal route of administration, for example, intranasally, vaginally, rectally, sublingually or topically. Administration can also be performed, for example, once, a plurality of times, and/or over one or more extended periods.

The terms “baseline” or “baseline value” used interchangeably herein can refer to a measurement or characterization of a symptom before the administration of the therapy or at the beginning of administration of the therapy. The baseline value can be compared to a reference value in order to determine the reduction or improvement of a symptom of a disease contemplated herein (e.g., breast cancer). The terms “reference” or “reference value” used interchangeably herein can refer to a measurement or characterization of a symptom after administration of the therapy. The reference value can be measured one or more times during a dosage regimen or treatment cycle or at the completion of the dosage regimen or treatment cycle. A “reference value” can be an absolute value; a relative value; a value that has an upper and/or lower limit; a range of values; an average value; a median value: a mean value; or a value as compared to a baseline value.

Similarly, a “baseline value” can be an absolute value; a relative value; a value that has an upper and/or lower limit; a range of values; an average value; a median value; a mean value; or a value as compared to a reference value. The reference value and/or baseline value can be obtained from one individual, from two different individuals or from a group of individuals (e.g., a group of two, three, four, five or more individuals).

An “adverse event” (AE) as used herein is any unfavorable and generally unintended or undesirable sign (including an abnormal laboratory finding), symptom, or disease associated with the use of a medical treatment. A medical treatment can have one or more associated AEs and each AE can have the same or different level of severity. Reference to methods capable of “altering adverse events” means a treatment regime that decreases the incidence and/or severity of one or more AEs associated with the use of a different treatment regime.

A “serious adverse event” or “SAE” as used herein is an adverse event that meets one of the following criteria:
Is fatal or life-threatening (as used in the definition of a serious adverse event, “life-threatening” refers to an event in which the patient was at risk of death at the time of the event; it does not refer to an event which hypothetically might have caused death if it was more severe.
Results in persistent or significant disability/incapacity
Constitutes a congenital anomaly/birth defect
Is medically significant, i.e., defined as an event that jeopardizes the patient or may require medical or surgical intervention to prevent one of the outcomes listed above. Medical and scientific judgment must be exercised in deciding whether an AE is “medically significant”
Requires inpatient hospitalization or prolongation of existing hospitalization, excluding the following: 1) routine treatment or monitoring of the underlying disease, not associated with any deterioration in condition; 2) elective or pre-planned treatment for a pre-existing condition that is unrelated to the indication under study and has not worsened since signing the informed consent; and 3) social reasons and respite care in the absence of any deterioration in the patient's general condition.

The terms “once about every week,” “once about every two weeks,” or any other similar dosing interval terms as used herein mean approximate numbers. “Once about every week” can include every seven days±one day, i.e., every six days to every eight days. “Once about every two weeks” can include every fourteen days±two days, i.e., every twelve days to every sixteen days. “Once about every three weeks” can include every twenty-one days±three days, i.e., every eighteen days to every twenty-four days. Similar approximations apply, for example, to once about every four weeks, once about every five weeks, once about every six weeks, and once about every twelve weeks. In some embodiments, a dosing interval of once about every six weeks or once about every twelve weeks means that the first dose can be administered any day in the first week, and then the next dose can be administered any day in the sixth or twelfth week, respectively. In other embodiments, a dosing interval of once about every six weeks or once about every twelve weeks means that the first dose is administered on a particular day of the first week (e.g., Monday) and then the next dose is administered on the same day of the sixth or twelfth weeks (i.e., Monday), respectively.

As described herein, any concentration range, percentage range, ratio range, or integer range is to be understood to include the value of any integer within the recited range and, when appropriate, fractions thereof (such as one tenth and one hundredth of an integer), unless otherwise indicated.

Various aspects of the disclosure are described in further detail in the following herein.

### II. Description of the Embodiments

A. Methods for Treating Cancer with Tucatinib in Combination with Trastuzumab, a Taxane, and a VEGFR-2 Antagonist

HER2 is a validated target in multiple solid tumors, with anti-HER2 biologics and small-molecule drugs approved for patients with HER2+ breast and gastric cancers. Amplification of the HER2-gene or overexpression of its protein occurs in approximately 15% to 20% of breast cancers and 6% to 30% of gastric and esophageal cancers. Recently, interest has grown in HER2-targeting strategies for patients with refractory metastatic colorectal carcinoma (CRC), where overexpression of HER2 has been found to occur in approximately 3% to 5% of patients. HER2 can also be overexpressed in other gastrointestinal cancers, such as cholangiocarcinoma and gallbladder carcinoma, where studies suggest ERBB2 amplification ranges from 1% to 6%.

The current standard of care for patients with HER2+ metastatic disease consists of treatment with pertuzumab plus trastuzumab and a taxane as first-line treatment for metastatic disease, followed by T-DM1 in second line. Treatment options for patients who progress after treatment with both pertuzumab and T-DM1 remain relatively limited. Patients are generally treated with a continuation of anti-HER2 therapy (in the form of trastuzumab or lapatinib) in combination with cytotoxic chemotherapy, such as capecitabine. Combined HER2 therapy with trastuzumab and lapatinib can also be considered. In some HER2 positive gastrointestional cancers, the standard of care of treatment with trastuzumab and chemotherapy, while treatment with lapatinib has been relatively ineffective against gastrointestional cancers. In other HER2 positive cancers such as gastroesophageal, colorectal, biliary tract, and gallbladder cancers, the standard of care is an oxaliplatin-based chemotherapy comprising the combination of oxaliplatin, fluorouracil, and leucovorin (e.g., FOLFOX and/or a modified FOLFOX regimen). However, better options for these patients are needed. The treatment and prevention of HER2 positive cancers represents an unmet need.

In some aspects, the disclosure provides a method of treating a HER2 positive cancer in a subject in need thereof, the method comprising administering to the subject a therapeutically effective amount of a combination therapy comprising tucatinib, trastuzumab, a taxane, and a vascular endothelial growth factor receptor 2 (VEGFR-2) antagonist (e.g., tucatinib, trastuzumab, paclitaxel, and ramucirumab).

In some aspects, the disclosure provides a method of treating a HER2 positive cancer in a subject in need thereof, the method comprising administering to the subject a therapeutically effective amount of a combination therapy comprising tucatinib, trastuzumab, paclitaxel, and ramucirumab.

In some aspects, the disclosure provides a method of treating cancer in a subject in need thereof comprising, identifying the subject as having a HER2 positive cancer. For example, the subject can have a HER2 positive cancer that is histologically or cytologically confirmed. In some embodiments, the HER2 positive cancer is selected from the group consisting of gastric adenocarcinoma, gastroesophageal junction (GEJ) adenocarcinoma, esophageal adenocarcinoma, colorectal carcinoma (CRC), cholangiocarcinoma, gallbladder carcinoma, gastric cancer, lung cancer, biliary cancers, bladder cancer, esophageal cancer, melanoma, ovarian cancer, liver cancer, prostate cancer, pancreatic cancer, small intestine cancer, non-small cell lung cancer, head and neck cancer, uterine cancer, cervical cancer, brain cancer, and breast cancer. In some embodiments, the HER2 positive cancer is selected from the group consisting of gastric adenocarcinoma, gastroesophageal junction (GEJ) adenocarcinoma, esophageal adenocarcinoma, colorectal carcinoma (CRC), cholangiocarcinoma, gallbladder carcinoma, gastric cancer, lung cancer, biliary cancers, bladder cancer, esophageal cancer, melanoma, ovarian cancer, liver cancer, prostate cancer, pancreatic cancer, small intestine cancer, non-small cell lung cancer, head and neck cancer, uterine cancer, cervical cancer, and brain cancer. In some embodiments, the HER2 positive cancer is unresectable or metastatic. In some embodiments, the method can further include administering to the subject a therapeutically effective amount of a combination therapy comprising tucatinib, trastuzumab, a taxane, and a VEGFR-2 antagonist (e.g., tucatinib, trastuzumab, paclitaxel, and ramucirumab).

In some embodiments, method can include administering to the subject a therapeutically effective amount of a combination therapy comprising a biosimilar. In such embodiments, a trastuzumab biosimilar can be used in the combination therapy such that the combination therapy comprises tucatinib, a trastuzumab biosimilar, a taxane, and a VEGFR-2 antagonist.

In some embodiments, the taxane is selected from the group consisting of paclitaxel, docetaxel, cabazitaxel, larotaxel, BMS-184476, BMS-188797, BMS-275183, milataxel, ortaxel, TL-310, docosahexaenoic acid-paclitaxel (DHA-paclitaxel), nab paclitaxel, EndoTAG+paclitaxel, XRP9881, polymeric-micellar paclitaxel, RPR-109881A, a pharmaceutically acceptable salt or solvate thereof, and a combination thereof. In some embodiments, the taxane is selected from the group consisting of paclitaxel, docetaxel, and cabazitaxel. In some embodiments, the taxane is paclitaxel.

In some embodiments, the VEGFR-2 antagonist is selected from the group consisting of bevacizumab, ramucirumab, aflibercept, cetuximab, panitumumab, regorafenib, sunitinib, sorafenib, pazopanib, vandetanib, axitinib, cediranib, vatalanib, motesanib, lucatinib, intedanib, semaxanib, apatinib, lenvatinib, carbozantinib, and a combination thereof. In some embodiments, the VEGFR-2 is a monoclonal antibody. In some embodiments, the VEGFR-2 antagonist is a monoclonal antibody selected from the group consisting of bevacizumab, ramucirumab, aflibercept, cetuximab, panitumumab, and combinations thereof. In some embodiments, the monoclonal antibody is ramucirumab.

In some embodiments, the subject was previously treated with at least one anticancer therapy. In such embodiments, the subject may have been treated with at least one anticancer therapy selected from the group consisting of trastuzumab (or a trastuzumab biosimilar), lapatinib, trastuzumab and a taxane, pertuzumab, and combinations thereof. In some embodiments, the subject has been previously treated with one or more additional therapeutic agents for the cancer and did not respond to the treatment. In some embodiments, the subject has been previously treated with one or more additional therapeutic agents for the cancer and relapsed after the treatment. In some embodiments, the subject can be refractory or develop a brain metastasis during the previous anticancer therapy.

In some embodiments, the subject has been previously treated with a HER2-directed antibody. In some such embodiments, the subject may have locally-advanced unresectable or metastatic HER2+ GEC who received prior treatment with a HER2-directed antibody; further, the subject may have received at least one prior line of therapy in the advance disease setting.

In some embodiments, the subject has not been previously treated. In some embodiments, the subject has not been previously treated with an anti-HER2 and/or an anti-EGFR tyrosine kinase inhibitor. In some embodiments, the subject has not been treated with the anti-HER2/EGFR tyrosine kinase inhibitor selected from the group consisting of tucatinib, lapatinib, neratinib, or afatinib.

In some embodiments, the subject has not been previously treated with a HER2-directed antibody-drug conjugate. In some embodiments, the subject was not treated with an antibody-drug conjugate is selected from the group consisting of ado-trastuzumab (T-DM1) or trastuzumab deruxtecan (DS8201a).

In some embodiments, the subject may have not been previously treated with tucatinib. In some embodiments, the subject has not been previously treated with an anthracycline. In some embodiments, the subject was not previously treated with anthracycline selected from the group consisting of doxorubicin, epirubicin, mitoxantrone, idarubicin, liposomal doxorubicin, and combinations thereof.

In some embodiments, the combination therapy is administered in a 28-day cycle. In some embodiments, tucatinib is dosed at approximately the same time as the taxane. In some such embodiments, tucatinib is dosed at approximately the same time as the start of the taxane infusion.

For example, the tucatinib can be administered with the taxane on cycle 1 day 8. In some embodiments, the taxane is administered first, followed by trastuzumab (or a trastuzumab biosimilar), and ramucirumab. In some embodiments, the combination therapy is administered by IV.

In some embodiments, the combination therapy is administered in a 28-day cycle. In some embodiments, tucatinib is dosed at approximately the same time as paclitaxel. In some such embodiments, tucatinib is dosed at approximately the same time as the start of the paclitaxel infusion. For example, the tucatinib can be administered with the paclitaxel on cycle 1 day 8. In some embodiments, the paclitaxel is administered first, followed by trastuzumab (or a trastuzumab biosimilar), and ramucirumab. In some embodiments, the combination therapy is administered by IV.

In some embodiments, the tucatinib is administered to the subject at a dose of about 150 mg to about 650 mg. In some embodiments, the tucatinib is administered to the subject at a dose of about 300 mg. In some embodiments, the tucatinib is administered twice daily. In some embodiments, the tucatinib is administered to the subject orally. In some embodiments, the tucatinib is administered to the subject orally, twice daily, beginning on cycle 1 day 1 and onward.

In some embodiments, trastuzumab is dosed at 6 mg/kg. In some embodiments, the trastuzumab is administered to the subject via IV. In some embodiments, trastuzumab is administered at 6 mg/kg as a loading dose and is administered on cycle 1 day 1. In some embodiments, following the loading dose, the trastuzumab is administered at 4 mg/kg. In some embodiments, following the loading dose, the trastuzumab is administered via IV at 4 mg/kg on cycle 1 day 15 and then days 1 and 15 of each cycle thereafter. In some embodiments, the trastuzumab is a trastuzumab biosimilar.

In some embodiments, a VEGFR-2 antagonist is administered on days 1 and 15 of each cycle. In some embodiments, the VEGFR-2 antagonist is administered via IV. In some embodiments, the VEGFR-2 antagonist is ramucirumab. In some embodiments, ramucirumab is administered on Days 1 and 15 of each cycle. In some embodiments, the ramucirumab is administered at a dose of about 4 mg/kg to about 12 mg/kg. In some embodiments, ramucirumab is administered at a dose of about 8 mg/kg. In some embodiments, ramucirumab is administered by IV.

In some embodiments, a taxane is administered on Days 1, 8, and 15 of each cycle. In some embodiments, the taxane is selected from the group consisting of paclitaxel, docetaxel, and cabazitaxel. In some embodiments, the taxane is administered by IV. In some embodiments, the taxane is paclitaxel. In some embodiments, paclitaxel is administered on Days 1, 8, and 15 of each cycle. In some embodiments, the paclitaxel is administered to the subject at a dose of about 50 mg/m² to about 100 mg/m². In some embodiments, the paclitaxel is administered to the subject at a dose of about 80 mg/m². In some embodiments, paclitaxel is administered by IV.

In some embodiments, the subject has been previously treated with one or more additional therapeutic agents for the cancer and experienced disease progression during the treatment. In some embodiments, the one or more additional therapeutic agents is an HER2 directed antibody. In some embodiments, the subject has been previously treated with trastuzumab, or pertuzumab. In some embodiments, the subject has been previously treated with trastuzumab. In some embodiments, the subject has been previously treated with pertuzumab. In some embodiments, the subject has been previously treated with trastuzumab and pertuzumab.

In some embodiments, the subject has not been previously treated with another anticancer therapy for the cancer within the past 1 day, 2 days, 3 days, 4 days, 5 days, 6 days, 7 days, 10 days, 2 weeks, 3 weeks, 4 weeks, 6 weeks, 2 months, 3 months, 7 months, 8 months, 9 months, 10 months, 11 months, 12 months, 15 months, 18 months, 2 years, 3 years, 4 years, 5 years, 6 years, 7 years, 8 years, 9 years or 10 years prior to being administered the therapeutically effective amount of tucatinib, or salt or solvate thereof. In some embodiments, the subject has not been previously treated with another therapeutic agent for the cancer within the past 12 months prior to being administered the therapeutically effective amount of tucatinib, or salt or solvate thereof. In some embodiments, the subject has not been previously treated with another therapeutic agent for the cancer. In some embodiments, the subject has not been treated with another anticancer therapy within the past three weeks. In some embodiments, the subject has not been previously treated with lapatinib, neratinib, afatinib, or capecitabine. In some embodiments, the subject has not been previously treated with lapatinib. In some embodiments, the subject has not been previously treated with neratinib. In some embodiments, the subject has not been previously treated with afatinib. In some embodiments, the subject has not been previously treated with capecitabine. In some embodiments, the subject has not been previously treated with an anti-HER2 antibody-drug conjugate (e.g., ado-trastuzumab emtansine).

In some embodiments, the HER2 status of a sample cell is determined. The determination can be made before treatment (i.e., administration of a combination of tucatinib, trastuzumab, a taxane, and a VEGFR-2 antagonist) begins, during treatment, or after treatment has been completed. In some instances, determination of the HER2 status results in a decision to change therapy (e.g., adding, changing, or discontinuing the use of the combination of tucatinib, trastuzumab, a taxane, and a VEGFR-2 antagonist (e.g., tucatinib, trastuzumab, paclitaxel, and ramucirumab), discontinuing therapy altogether, or switching from another treatment method to a method of the present disclosure).

In some embodiments, the sample cell is determined to be overexpressing or not overexpressing HER2. In particular embodiments, the cell is determined to be HER2 3+, HER2 2+, HER2 1+, or HER2 0 (i.e., HER is not overexpressed).

In some embodiments, the sample cell is a cancer cell. In some instances, the sample cell is obtained from a subject who has cancer. The sample cell can be obtained as a biopsy specimen, by surgical resection, or as a fine needle aspirate (FNA). In some embodiments, the sample cell is a circulating tumor cell (CTC).

HER2 expression can be compared to a reference cell. In some embodiments, the reference cell is a non-cancer cell obtained from the same subject as the sample cell. In other embodiments, the reference cell is a non-cancer cell obtained from a different subject or a population of subjects. In some embodiments, measuring expression of HER2 comprises, for example, determining HER2 gene copy number or amplification, nucleic acid sequencing (e.g., sequencing of genomic DNA or cDNA), measuring mRNA expression, measuring protein abundance, or a combination thereof. HER2 testing methods include immunohistochemistry (IHC), in situ hybridization, fluorescence in situ hybridization (FISH), chromogenic in situ hybridization (CISH), ELISAs, and RNA quantification (e.g., of HER2 expression) using techniques such as RT-PCR and microarray analysis.

In some embodiments, the sample cell is determined to be HER2 positive when HER2 is expressed at a higher level in the sample cell compared to a reference cell. In some embodiments, the cell is determined to be HER2 positive when HER2 is overexpressed at least about 1.5-fold (e.g., about 1.5-fold, 2-fold, 2.5-fold, 3-fold, 3.5-fold, 4-fold, 4.5-fold, 5-fold, 5.5-fold, 6-fold, 6.5-fold, 7-fold, 7.5-fold, 8-fold, 8.5-fold, 9-fold, 9.5-fold, 10-fold, 11-fold, 12-fold, 13-fold, 14-fold, 15-fold, 16-fold, 17-fold, 18-fold, 19-fold, 20-fold, 25-fold, 30-fold, 35-fold, 40-fold, 45-fold, 50-fold, 55-fold, 60-fold, 65-fold, 70-fold, 75-fold, 80-fold, 85-fold, 90-fold, 95-fold, 100-fold, or more) compared to a reference cell. In particular embodiments, the cell is determined to be HER2 positive when HER2 is overexpressed at least about 1.5-fold compared to the reference cell.

In some embodiments, the sample cell is determined to be HER2 positive when the FISH or CISH signal ratio is greater than 2. In other embodiments, the sample cell is determined to be HER2 positive when the HER2 gene copy number is greater than 6.

In one aspect, provided herein are methods for treating or ameliorating a HER2 positive cancer in a subject in need thereof, the method comprising administering to the subject a therapeutically effective amount of a combination therapy comprising tucatinib, trastuzumab, a taxane, and a VEGFR-2 antagonist (e.g., tucatinib, trastuzumab, paclitaxel, and ramucirumab).

In one aspect provided herein are methods for treating a HER2 positive cancer in a subject that has exhibited an adverse event after starting treatment with a combination therapy comprising tucatinib, trastuzumab, a taxane, and a VEGFR-2 antagonist at an initial dosage level, comprising administering to the subject at least one of the combination therapy at a reduced dosage level. For example, one, two, or all of the components included in the combination therapy can be reduced. In such embodiments, an individual component (i.e., one of tucatinib, trastuzumab, a taxane, and a VEGFR-2 antagonist) of the combination therapy can be reduced following an adverse reaction experienced by the subject while the other components of the compound therapy remain at their initial dosage levels. In another such embodiment, two of the components (i.e., two of tucatinib, trastuzumab, a taxane, and a VEGFR-2 antagonist) of the combination therapy can be reduced following an adverse reaction experienced by the subject while the remaining component of the compound therapy remains at its initial dosage level. In another such embodiment, all of the components (i.e., tucatinib, trastuzumab, a taxane, and a VEGFR-2 antagonist) of the combination therapy can be reduced following an adverse reaction experienced by the subject and none of the components that comprise the compound therapy remain at the initial dosage level. For example, the methods can include methods for treating the HER2 positive cancer in a subject that has exhibited an adverse event after starting treatment with at least one of the combination therapy comprising tucatinib, trastuzumab, a taxane, and a VEGFR-2 antagonist.

In some aspects, provided herein are methods for treating a HER2 positive cancer in a subject that has exhibited an adverse event after starting treatment with a combination therapy comprising tucatinib, trastuzumab, a taxane, and a VEGFR-2 antagonist at an initial dosage level, comprising administering to the subject at least one component of the combination therapy at a reduced dosage level. In some embodiments, the taxane is paclitaxel. In some embodiments, the VEGFR-2 is ramucirumab.

In such embodiments, the paclitaxel is administered to the subject at an initial dose of about 50 mg/m² to about 100 mg/m². In some embodiments, the paclitaxel is administered to the subject at an initial dose of about 80 mg/m². In some embodiments, the paclitaxel is administered to the subject at a reduced dose of about 50 mg/m² to about 75 mg/m². In some embodiments, the paclitaxel is administered to the subject at a reduced dose of about 70 mg/m². In some embodiments, the paclitaxel is administered to the subject at a reduced dose of about 60 mg/m². In some embodiments, the tucatinib is administered to the subject at an initial dose of about 150 mg to about 650 mg. In such embodiments, the tucatinib is administered to the subject at an initial dose of about 300 mg. In some embodiments, the tucatinib is administered to the subject at a reduced dose of about 125 mg to about 275 mg. In some embodiments, wherein the ramucirumab is administered to the subject at an initial dose of about 8 mg/kg. In some embodiments, the ramucirumab is administered to the subject at a reduced dose of about 6 mg/kg. In some embodiments, the ramucirumab is administered to the subject at a reduced dose of about 5 mg/kg.

In some embodiments of any of the methods described herein, the method can further comprise treating a HER2 positive cancer in a subject in need thereof comprising administering to the subject a therapeutically effective amount of a combination therapy comprising tucatinib, trastuzumab, a taxane, and a VEGFR-2 antagonist, and administering an effective amount of an anti-diarrheal agent.

In some embodiments, the anti-diarrheal agent is administered prophylactically. In some embodiments of any of the methods described herein, the method can include reducing the severity or incidents of diarrhea, or preventing diarrhea in a subject having a HER2 positive cancer and being treated with an effective amount of a combination therapy comprising tucatinib, trastuzumab, a taxane, and a VEGFR-2 antagonist, the method comprising administering an effective amount of an anti-diarrheal agent prophylactically.

In another embodiment of any of the methods described herein, the method can include a method of reducing the likelihood of a subject developing diarrhea, wherein the subject has a HER2 positive cancer and is being treated with an effective amount of a combination therapy comprising tucatinib, trastuzumab, a taxane, and a VEGFR-2 antagonist the method comprising administering an effective amount of an anti-diarrheal agent prophylactically.

In some embodiments, the combination therapy and the anti-diarrheal agent are administered sequentially. In some embodiments, the combination therapy and the anti-diarrheal agent are administered concurrently. In some embodiments, the anti-diarrheal agent is administered prior to administration of the combination therapy. For example, one hour before, two hours before, four hours before, six hours before, twelve hours before, one day before, two days before, three days before, four days before, five days before, or one week before. In some cases, the subject is exhibiting symptoms of diarrhea prior to administration of the anti-diarrheal agent. In other cases, the subject is not exhibiting symptoms of diarrhea prior to administration of the anti-diarrheal agent.

Non-limiting examples of anti-diarrheal agents include loperamide, budesonide (e.g., in combination with loperamide), prophylactic antibiotics (e.g., doxycycline), probiotics, electrolyte replacement solutions, colestipol, colestipol in combination with loperamide, octreotide, crofelemer, TJ14, Bacillus Cereus, calcium aluminosilicate, sulfasalazine, cefpodoxime, elsiglutide, glutamine, codeine, diphenoxylate, atropine, bismuth subsalicylate, diphenoxylate, atropine, attapulgite, activated charcoal, bentonite, Saccharomyces boulardii lyo, rifaximin, neomycin, alosetron, octreotide, crofelemer, opium, cholestyramine, and colesevelam.

In some embodiments, the combination therapy and the anti-diarrheal agent are administered sequentially. In some embodiments, the combination therapy and the anti-diarrheal agent are administered concurrently. In some embodiments, the anti-diarrheal agent is administered prior to administration of the combination therapy. For example, one hour before, two hours before, four hours before, six hours before, twelve hours before, one day before, two days before, three days before, four days before, five days before, or one week before. In some cases, the subject is exhibiting symptoms of diarrhea prior to administration of the antiemetic agent. In other cases, the subject is not exhibiting symptoms of diarrhea prior to administration of the antiemetic agent.

### B. Tucatinib Dose and Administration

In some embodiments, a dose of tucatinib is between about 0.1 mg and 10 mg per kg of the subject's body weight (e.g., about 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, or 10 mg per kg of the subject's body weight). In other embodiments, a dose of tucatinib is between about 10 mg and 100 mg per kg of the subject's body weight (e.g., about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, or 100 mg per kg of the subject's body weight). In some embodiments, a dose of tucatinib is at least about 100 mg to 500 mg per kg of the subject's body weight (e.g., at least about 100, 125, 150, 175, 200, 225, 250, 275, 300, 325, 350, 375, 400, 425, 450, 475, or 500 mg per kg of the subject's body weight). In particular embodiments, a dose of tucatinib is between about 1 mg and 50 mg per kg of the subject's body weight (e.g., about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 mg per kg of the subject's body weight). In some instances, a dose of tucatinib is about 50 mg per kg of the subject's body weight.

In some embodiments, a dose of tucatinib comprises between about 1 mg and 100 mg (e.g. about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, or 100 mg) of tucatinib. In other embodiments, a dose of tucatinib comprises between about 100 mg and 1,000 mg (e.g., about 100, 105, 110, 115, 120, 125, 130, 135, 140, 145, 150, 155, 160, 165, 170, 175, 180, 185, 190, 195, 200, 205, 210, 215, 220, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, or 1,000 mg) of tucatinib. In particular embodiments, a dose of tucatinib is about 300 mg (e.g., when administered twice per day). In certain of these embodiments, a dose of tucatinib is 300 mg (e.g., 6×50 mg tablets; or 2×150 mg tablets), administered twice per day.

In some embodiments, a dose of tucatinib comprises at least about 1,000 mg to 10,000 mg (e.g., at least about 1,000, 1,100, 1,200, 1,300, 1,400, 1,500, 1,600, 1,700, 1,800, 1,900, 2,000, 2,100, 2,200, 2,300, 2,400, 2,500, 2,600, 2,700, 2,800, 2,900, 3,000, 3,100, 3,200, 3,300, 3,400, 3,500, 3,600, 3,700, 3,800, 3,900, 4,000, 4,100, 4,200, 4,300, 4,400, 4,500, 4,600, 4,700, 4,800, 4,900, 5,000, 5,100, 5,200, 5,300, 5,400, 5,500, 5,600, 5,700, 5,800, 5,900, 6,000, 6,100, 6,200, 6,300, 6,400, 6,500, 6,600, 6,700, 6,800, 6,900, 7,000, 7,100, 7,200, 7,300, 7,400, 7,500, 7,600, 7,700, 7,800, 7,900, 8,000, 8,100, 8,200, 8,300, 8,400, 8,500, 8,600, 8,700, 8,800, 8,900, 9,000, 9,100, 9,200, 9,300, 9,400, 9,500, 9,600, 9,700, 9,800, 9,900, 10,000 or more mg) of tucatinib.

In some embodiments, a dose of tucatinib, or salt or solvate thereof, contains a therapeutically effective amount of tucatinib, or salt or solvate thereof. In other embodiments, a dose of tucatinib, or salt or solvate thereof, contains less than a therapeutically effective amount of tucatinib, or salt or solvate thereof, (e.g., when multiple doses are given in order to achieve the desired clinical or therapeutic effect).

Tucatinib, or salt or solvate thereof, can be administered by any suitable route and mode. Suitable routes of administering combination therapies of the present disclosure are well known in the art and may be selected by those of ordinary skill in the art. In one embodiment, tucatinib administered parenterally. Parenteral administration refers to modes of administration other than enteral and topical administration, usually by injection, and include epidermal, intravenous, intramuscular, intraarterial, intrathecal, intracapsular, intraorbital, intracardiac, intradermal, intraperitoneal, intratendinous, transtracheal, subcutaneous, subcuticular, intraarticular, subcapsular, subarachnoid, intraspinal, intracranial, intrathoracic, epidural and intrasternal injection and infusion. In some embodiments, the route of administration of tucatinib is intravenous injection or infusion. In some embodiments, the route of administration of tucatinib is intravenous infusion. In some embodiments, the route of administration of tucatinib is intravenous injection or infusion. In some embodiments, the tucatinib is intravenous infusion. In some embodiments, the route of administration of tucatinib is oral.

In one embodiment of the methods or uses or product for uses provided herein, tucatinib is administered to the subject at a dose of about 150 mg to about 650 mg.

In one embodiment of the methods or uses or product for uses provided herein, tucatinib is administered to the subject daily, twice daily, three times daily or four times daily. In some embodiments, tucatinib is administered to the subject every other day, once about every week or once about every three weeks. In some embodiments, tucatinib is administered to the subject once per day. In some embodiments, tucatinib is administered to the subject twice per day. In some embodiments, tucatinib is administered to the subject at a dose of about 300 mg twice per day. In some embodiments, tucatinib is administered to the subject at a dose of 300 mg twice per day. In some embodiments, tucatinib is administered to the subject at a dose of about 600 mg once per day. In some embodiments, tucatinib is administered to the subject at a dose of 600 mg once per day. In some embodiments, tucatinib is administered to the subject twice per day on each day of a 21 day treatment cycle. In some embodiments, the tucatinib is administered to the subject orally.

### C. Trastuzumab Dose and Administration

In some embodiments, trastuzumab (or a biosimilar thereof) is administered on Day 1 and 15 of every 28-day cycle. In some embodiments, trastuzumab is administered as a loading dose. In some embodiments, a loading dose of about 6 mg/kg is administered. In some embodiments, the loading dose is administered via IV. In some embodiments, the loading dose is administered on cycle 1 day 1. In some embodiments, following the loading dose, the trastuzumab is dosed at 4 mg/kg with each subsequent dose. In some embodiments, the trastuzumab is administered on a weekly basis. In some embodiments, the trastuzumab is administered at 2 mg/kg via IV once weekly.

### D. VEGFR-2 Antagonist Dose and Administration

In some embodiments, a VEGFR-2 antagonist (e.g., ramucirumab) is administered on Days 1 and 15 of each 28 day cycle. In some embodiments, the VEGFR-2 antagonist is ramucirumab. In some embodiments, ramucirumab is administered on Days 1 and 15 of each 28 day cycle. In some embodiments, ramucirumab is administered at a dose of about 8 mg/kg on Days 1 and 15 of each 28-day cycle. In some embodiments, the VEGFR-2 antagonist is administered via IV. In some embodiments, ramucirumab is administered via IV.

### E. Taxane Dose and Administration

In some embodiments, a taxane (e.g., paclitaxel) is administered on Days 1, 8, and 15 of each cycle. In some embodiments, the taxane is selected from the group consisting of paclitaxel, docetaxel, and cabazitaxel. In some embodiments, the taxane is paclitaxel. In some embodiments, paclitaxel is administered at a dose of about 80 mg/m². In some embodiments, the paclitaxel is administered on Days 1, 8, and 15 of each 28-day cycle. In some embodiments, a taxane is administered via IV. In some embodiments, paclitaxel is administered via IV. In some embodiments, paclitaxel is administered at a dose of about 80 mg/m² is on Days 1, 8, and 15 of each 28-day cycle.

### F. Combination Therapy

Provided herein are methods of treatment comprising administering to the subject a combination therapy comprising tucatinib, trastuzumab, a taxane, and a VEGFR-2 antagonist (e.g., tucatinib, trastuzumab, paclitaxel, and ramucirumab). In some embodiments, the combination therapy consists essentially of tucatinib, trastuzumab, a taxane, and a VEGFR-2 antagonist (e.g., tucatinib, trastuzumab, paclitaxel, and ramucirumab).

In some embodiments, the tucatinib, trastuzumab, a taxane, and a VEGFR-2 antagonist are administered to the subject on a treatment cycle. In some embodiments, the tucatinib, trastuzumab, a taxane, and a VEGFR-2 antagonist are administered to a subject on a 28 day treatment cycle. In some embodiments, the subject will be treated with tucatinib, trastuzumab, a taxane selected from paclitaxel, docetaxel, cabazitaxel, larotaxel, BMS-184476, BMS-188797, BMS-275183, milataxel, ortaxel, TL-310, docosahexaenoic acid-paclitaxel (DHA-paclitaxel), nab paclitaxel, EndoTAG+paclitaxel, XRP9881, polymeric-micellar paclitaxel, RPR-109881A, a pharmaceutically acceptable salt or solvate thereof, and a combination thereof, and a VEGFR-2 antagonist that is selected from the group consisting of bevacizumab, ramucirumab, aflibercept, cetuximab, panitumumab, regorafenib, sunitinib, sorafenib, pazopanib, vandetanib, axitinib, cediranib, vatalanib, motesanib given in 28-day cycles. For instance, the subject can be treated with tucatinib, trastuzumab, a taxane, and a VEGFR-2 antagonist given in 28-day cycles. An eligible subject may have received prior treatment of a HER2-direted antibody prior to receiving the combination treatment. The starting dose of tucatinib is 300 mg orally (PO) BID, and the first dose will be administered on cycle 1 day 8 and continuously thereafter.

### G. Compositions

In another aspect, the present disclosure provides a pharmaceutical composition comprising tucatinib, trastuzumab, a taxane, and a VEGFR-2 antagonist (e.g., tucatinib, trastuzumab, paclitaxel, and ramucirumab), and a pharmaceutically acceptable carrier. In another aspect, the present disclosure provides a pharmaceutical combination comprising tucatinib, trastuzumab, a taxane, and a VEGFR-2 antagonist, and a pharmaceutically acceptable carrier. In some examples, the tucatinib can be administered separately from the trastuzumab, the taxane and the VEGFR-2 antagonist. In other examples, the tucatinib can be administered with one, two, or all of the trastuzumab, taxane, and/or the VEGFR-2 antagonist.

The pharmaceutical compositions of the present disclosure may be prepared by any of the methods well-known in the art of pharmacy. Pharmaceutically acceptable carriers suitable for use with the present disclosure include any of the standard pharmaceutical carriers, buffers and excipients, including phosphate-buffered saline solution, water, and emulsions (such as an oil/water or water/oil emulsion), and various types of wetting agents or adjuvants. Suitable pharmaceutical carriers and their formulations are described in Remington's Pharmaceutical Sciences (Mack Publishing Co., Easton, 19th ed. 1995). Preferred pharmaceutical carriers depend upon the intended mode of administration of the active agent.

The pharmaceutical compositions of the present disclosure can include a combination of drugs (e.g., tucatinib, trastuzumab, a taxane, and a VEGFR-2 antagonist), or any pharmaceutically acceptable salts thereof, as active ingredients and a pharmaceutically acceptable carrier or excipient or diluent. A pharmaceutical composition may optionally contain other therapeutic ingredients.

The compositions (e.g., tucatinib, trastuzumab, a taxane, and a VEGFR-2 antagonist) can be combined as the active ingredients in intimate admixture with a suitable pharmaceutical carrier or excipient according to conventional pharmaceutical compounding techniques. Any carrier or excipient suitable for the form of preparation desired for administration is contemplated for use with the compounds disclosed herein.

The pharmaceutical compositions include those suitable for oral, topical, parenteral, pulmonary, nasal, or rectal administration. The most suitable route of administration in any given case will depend in part on the nature and severity of the cancer condition and also optionally the HER2 status or stage of the cancer.

Other pharmaceutical compositions include those suitable for systemic (e.g., enteral or parenteral) administration. Systemic administration includes oral, rectal, sublingual, or sublabial administration. Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. In particular embodiments, pharmaceutical compositions of the present disclosure may be administered intratumorally.

Compositions for systemic administration include, but are not limited to, dry powder compositions consisting of the composition as set forth herein (e.g., tucatinib, trastuzumab, a taxane, and a VEGFR-2 antagonist) and the powder of a suitable carrier or excipient. The compositions for systemic administration can be represented by, but not limited to, tablets, capsules, pills, syrups, solutions, and suspensions.

In some embodiments, the compositions (e.g., tucatinib, trastuzumab, a taxane, and a VEGFR-2 antagonist) further include a pharmaceutical surfactant. In other embodiments, the compositions further include a cryoprotectant. In some embodiments, the cryoprotectant is selected from the group consisting of glucose, sucrose, trehalose, lactose, sodium glutamate, PVP, HPPCD, CD, glycerol, maltose, mannitol, and saccharose.

Pharmaceutical compositions or medicaments for use in the present disclosure can be formulated by standard techniques using one or more physiologically acceptable carriers or excipients. Suitable pharmaceutical carriers are described herein and in Remington: The Science and Practice of Pharmacy, 21st Ed., University of the Sciences in Philadelphia, Lippencott Williams & Wilkins (2005).

### Pharmaceutical Compositions of Tucatinib

In some embodiments, a pharmaceutical composition comprising tucatinib and a pharmaceutically acceptable carrier is provided herein, wherein the pharmaceutical composition comprises a solid dispersion of tucatinib.

The solid dispersions are generally prepared by dissolving the drug substance and the dispersion polymer in a suitable solvent to form a feed solution, and then the feed solution may be spray dried to form the solid dispersion (and remove the solvent). Spray drying is a known process. Spray drying is generally performed by dissolving tucatinib and the dispersion polymer in a suitable solvent to prepare a feed solution. The feed solution may be pumped through an atomizer into a drying chamber. The feed solution can be atomized by conventional means known in the art, such as a two-fluid sonicating nozzle, a pressure nozzle, a rotating nozzle and a two-fluid non-sonicating nozzle. Then, the solvent is removed in the drying chamber to form the solid dispersion. A typical drying chamber uses hot gases, such as forced air, nitrogen, nitrogen-enriched air, or argon to dry particles. The size of the drying chamber may be adjusted to achieve particle properties or throughput. Although the solid dispersion are preferably prepared by conventional spray drying techniques, other techniques known in the art may be used, such as melt extrusion, freeze drying, rotary evaporation, drum drying or other solvent removal processes.

In some embodiments, a process of preparing a solid dispersion is provided, comprising: (a) dissolving tucatinib and a dispersion polymer in a suitable solvent; and (b) evaporating the solvent to form the solid dispersion. In certain embodiments, the evaporation of the solvent in step (b) is performed by spray drying, melt extrusion, freeze drying, rotary evaporation, drum drying or other solvent removal processes.

In certain embodiments, the dispersion polymer is selected from PVP-VA, methylacrylic acid methyl methacrylate copolymer, HPMCP, CAP, HPMCAS and HPMC and mixtures thereof. In certain embodiments, the dispersion polymer is selected from PVP-VA, methylacrylic acid methyl methacrylate copolymer, HPMCP, CAP, HPMCAS and HPMC. In certain embodiments, the dispersion polymer is selected from PVP-VA, Eudragit® L100, HPMCP H-55, CAP, HPMCAS Grade M, HPMC and mixtures thereof. In certain embodiments, the dispersion polymer is selected from PVP-VA, Eudragit® L100, HPMCP H-55, CAP, HPMCAS Grade M and HPMC.

In certain embodiments, the dispersion polymer is selected from PVP-VA, methylacrylic acid methyl methacrylate copolymer, HPMCP, CAP and HPMCAS, and mixtures thereof. In certain embodiments, the dispersion polymer is selected from PVP-VA, methylacrylic acid methyl methacrylate copolymer, HPMCP, CAP and HPMCAS. In certain embodiments, the dispersion polymer is selected from PVP-VA, Eudragit® L100, HPMCP H-55, CAP and HPMCAS Grade M, and mixtures thereof. In certain embodiments, the dispersion polymer is selected from PVP-VA, Eudragit® L100, HPMCP H-55, CAP and HPMCAS Grade M.

In certain embodiments, the dispersion polymer is selected from PVP-VA, methylacrylic acid methyl methacrylate copolymer, HPMCP, CAP and HPMC, and mixtures thereof. In certain embodiments, the dispersion polymer is selected from PVP-VA, methylacrylic acid methyl methacrylate copolymer, HPMCP, CAP and HPMC. In certain embodiments, the dispersion polymer is selected from PVP-VA, Eudragit® L100, HPMCP H-55, CAP and HPMC, and mixtures thereof. In certain embodiments, the dispersion polymer is selected from PVP-VA, Eudragit® L100, HPMCP H-55, CAP and HPMC

In certain embodiments, the dispersion polymer is selected from PVP-VA, methylacrylic acid methyl methacrylate copolymer, HPMCP and CAP, and mixtures thereof. In certain embodiments, the dispersion polymer is selected from PVP-VA, methylacrylic acid methyl methacrylate copolymer, HPMCP and CAP. In certain embodiments, the dispersion polymer is selected from PVP-VA, Eudragit® L100, HPMCP H-55 and CAP, and mixtures thereof. In certain embodiments, the dispersion polymer is selected from PVP-VA, Eudragit® L100, HPMCP H-55 and CAP. In certain embodiments, the dispersion polymer is PVP-VA.

In certain embodiments, the dispersion polymer is methylacrylic acid methyl methacrylate copolymer. In certain embodiments, the dispersion polymer is Eudragit®. In certain embodiments, the dispersion polymer is Eudragit® L100.

In certain embodiments, the dispersion polymer is HPMCP. In certain embodiments, the dispersion polymer is HPMCP H-55.

In certain embodiments, the dispersion polymer is CAP.

In certain embodiments, the dispersion polymer is HPMCAS. In certain embodiments, the dispersion polymer is HPMCAS Grade M.

In certain embodiments, the dispersion polymer is preferably neutral or basic.

In certain embodiments, the dispersion polymer is selected from PVP-VA and HPMC. In certain embodiments, the dispersion polymer is HPMC.

Suitable solvents are a solvent or mixture of solvents in which both tucatinib and the dispersion polymer have adequate solubility (solubility greater than 1 mg/mL). A mixture of solvents may be used if each component of the solid dispersion (i.e., tucatinib and dispersion polymer) require different solvents to obtain the desired solubility. The solvent may be volatile with a boiling point of 150° C. or less. In addition, the solvent should have relatively low toxicity and be removed from the dispersion to a level that is acceptable to The International Committee on Harmonization (“ICH”) guidelines. Removal of solvent to this level may require a subsequent processing step, such as tray drying. Examples of suitable solvents include, but are not limited to, alcohols, such as methanol (“MeOH”), ethanol (“EtOH”), n-propanol, isopropanol (“IPA”) and butanol; ketones, such as acetone, methyl ethyl ketone (“MEK”) and methyl isobutyl ketone; esters, such as ethyl acetate (“EA”) and propyl acetate; and various other solvents, such as tetrahydrofuran (“THF”), acetonitrile (“ACN”), methylene chloride, toluene and 1,1,1-trichloroethane. Lower volatility solvents, such as dimethyl acetate or dimethylsulfoxide (“DMSO”), may be used. Mixtures of solvents with water may also be used, so long as the polymer and tucatinib are sufficiently soluble to make the spray drying process practicable. Generally, due to the hydrophobic nature of low solubility drugs, non-aqueous solvents may be used, meaning the solvent comprises less than about 10 weight % water.

In certain embodiments, the suitable solvent is selected from MeOH and THF, and mixtures thereof. In certain embodiments, the suitable solvent is MeOH:THF solvent system of about 1:3. In certain embodiments, the suitable solvent is a 1:3 MeOH:THF solvent system.

In certain embodiments, the suitable solvent is selected from MeOH, THE and water, and mixtures thereof. In certain embodiments, the suitable solvent is selected from MeOH, THE and water. In certain embodiments, the suitable solvent is a THF:MeOH:water solvent system of about 80:10:10. In certain embodiments, the suitable solvent is a 80:10:10 THF:MeOH:water solvent system. In certain embodiments, the suitable solvent is a THF:MeOH:water solvent system of about 82:8:10. In certain embodiments, the suitable solvent is a 82:8:10 THF:MeOH:water solvent system. In certain embodiments, the suitable solvent is a THF:MeOH:water solvent system of about 82.2:8.2:9.6. In certain embodiments, the suitable solvent is a 82.2:8.2:9.6 THF:MeOH:water solvent system.

In certain embodiments, the amount of tucatinib in the solid dispersion ranges from about 0.1% to about 70% by weight relative to the dispersion polymer. In certain embodiments, the amount of tucatinib in the solid dispersion ranges from 0.1% to 70% by weight relative to the dispersion polymer.

In certain embodiments, the amount of tucatinib in the solid dispersion ranges from about 1% to about 60% by weight relative to the dispersion polymer. In certain embodiments, the amount of tucatinib in the solid dispersion ranges from 1% to 60% by weight relative to the dispersion polymer.

In certain embodiments, the amount of tucatinib in the solid dispersion ranges from about 5% to about 60% by weight relative to the dispersion polymer. In certain embodiments, the amount of tucatinib in the solid dispersion ranges from 5% to 60% by weight relative to the dispersion polymer.

In certain embodiments, the amount of tucatinib in the solid dispersion ranges from about 55% to about 65% by weight relative to the dispersion polymer. In certain embodiments, the amount of tucatinib in the solid dispersion ranges from 55% to 65% by weight relative to the dispersion polymer. In certain embodiments, the amount of tucatinib in the solid dispersion is about 60% by weight relative to the dispersion polymer. In certain embodiments, the amount of tucatinib in the solid dispersion is 60% by weight relative to the dispersion polymer.

In certain embodiments, the amount of tucatinib in the solid dispersion ranges from about 25% to about 35% by weight relative to the dispersion polymer. In certain embodiments, the amount of tucatinib in the solid dispersion ranges from 25% to 35% by weight relative to the dispersion polymer. In certain embodiments, the amount of tucatinib in the solid dispersion is about 30% by weight relative to the dispersion polymer. In certain embodiments, the amount of tucatinib in the solid dispersion is 30% by weight relative to the dispersion polymer.

In certain embodiments, the amount of tucatinib in the solid dispersion ranges from about 45% to about 55% by weight relative to the dispersion polymer. In certain embodiments, the amount of tucatinib in the solid dispersion ranges from 45% to 55% by weight relative to the dispersion polymer. In certain embodiments, the amount of tucatinib in the solid dispersion is about 50% by weight relative to the dispersion polymer. In certain embodiments, the amount of tucatinib in the solid dispersion is 50% by weight relative to the dispersion polymer.

In certain embodiments, the solid dispersion is an amorphous solid dispersion.

Another embodiment provides a pharmaceutical composition comprising a solid dispersion of tucatinib and a dispersion polymer, and a carrier or excipient.

Suitable carriers and excipients are well known to those skilled in the art and are described in detail in, e.g., Ansel, Howard C., et al., Ansel's Pharmaceutical Dosage Forms and Drug Delivery Systems. Philadelphia: Lippincott, Williams & Wilkins, 2004; Gennaro, Alfonso R., et al. Remington: The Science and Practice of Pharmacy. Philadelphia: Lippincott, Williams & Wilkins, 2000; and Rowe, Raymond C. Handbook of Pharmaceutical Excipients. Chicago, Pharmaceutical Press, 2005.

The pharmaceutical compositions may also include one or more additional components, such as buffers, dispersion agents, surfactants, wetting agents, lubricating agents, emulsifiers, suspending agents, preservatives, antioxidants, opaquing agents, glidants, processing aids, colorants, sweeteners, perfuming agents, flavoring agents, diluents and other known additives to provide an elegant presentation of the drug, i.e., a compound described herein or pharmaceutical composition thereof, or aid in the manufacturing of the pharmaceutical product, i.e., medicament (see Ansel; Gennaro; and Rowe above). The components of the pharmaceutical composition should be pharmaceutically acceptable.

Certain embodiments provide a pharmaceutical composition comprising: (a) about 1 to about 70 weight % of a solid dispersion of tucatinib; (b) about 0.1 to about 20 weight % of a disintegrant; (c) about 0.1 to about 25 weight % of an osmogen; (d) about 0.1 to about 10 weight % of a glidant; (e) about 0.1 to about 10 weight % of a lubricant; and (f) about 0.1 to about 25 weight % of a binder/diluent.

In certain embodiments, the pharmaceutical composition comprises: (a) 1 to 70 weight % of a solid dispersion of tucatinib; (b) 0.1 to 20 weight % of a disintegrant; (c) 0.1 to 25 weight % of an osmogen; (d) 0.1 to 10 weight % of a glidant; (e) 0.1 to 10 weight % of a lubricant; and (f) 0.1 to 25 weight % of a binder/diluent.

Certain embodiments provide a pharmaceutical composition comprising: (a) about 25 to about 60 weight % of a solid dispersion of tucatinib; (b) about 5 to about 15 weight % of a disintegrant; (c) about 15 to about 25 weight % of an osmogen; (d) about 0.1 to about 3 weight % of a glidant; (e) about 0.1 to about 3 weight % of a lubricant; and (f) about 10 to about 25 weight % of a binder/diluent.

In certain embodiments, the pharmaceutical composition comprises: (a) 25 to 60 weight % of a solid dispersion of tucatinib; (b) 5 to 15 weight % of a disintegrant; (c) 15 to 25 weight % of an osmogen; (d) 0.1 to 3 weight % of a glidant; (e) 0.1 to 3 weight % of a lubricant; and (f) 10 to 25 weight % of a binder/diluent.

Certain embodiments provide a pharmaceutical composition comprising: (a) about 40 to about 60 weight % of a solid dispersion of tucatinib; (b) about 5 to about 15 weight % of a disintegrant; (c) about 15 to about 25 weight % of an osmogen; (d) about 0.1 to about 3 weight % of a glidant; (e) about 0.1 to about 3 weight % of a lubricant; and (f) about 10 to about 25 weight % of a binder/diluent.

In certain embodiments, the pharmaceutical composition comprises: (a) 40 to 60 weight % of a solid dispersion of tucatinib; (b) 5 to 15 weight % of a disintegrant; (c) 15 to 25 weight % of an osmogen; (d) 0.1 to 3 weight % of a glidant; (e) 0.1 to 3 weight % of a lubricant; and (f) 10 to 25 weight % of a binder/diluent.

Certain embodiments provide a pharmaceutical composition comprising: (a) about 1 to about 70 weight % of a solid dispersion of tucatinib; (b) about 0.1 to about 20 weight % of a disintegrant; (c) about 0.1 to about 25 weight % of an osmogen; (d) about 0.1 to about 10 weight % of a glidant; (e) about 0.1 to about 10 weight % of a lubricant; and (f) about 0.1 to about 25 weight % of a filler.

In certain embodiments, the pharmaceutical composition comprises: (a) 1 to 70 weight % of a solid dispersion of tucatinib; (b) 0.1 to 20 weight % of a disintegrant; (c) 0.1 to 25 weight % of an osmogen; (d) 0.1 to 10 weight % of a glidant; (e) 0.1 to 10 weight % of a lubricant; and (f) 0.1 to 25 weight % of a filler.

Certain embodiments provide a pharmaceutical composition comprising: (a) about 25 to about 60 weight % of a solid dispersion of tucatinib; (b) about 1 to about 10 weight % of a disintegrant; (c) about 15 to about 25 weight % of an osmogen; (d) about 0.1 to about 3 weight % of a glidant; (e) about 0.1 to about 3 weight % of a lubricant; and (f) about 10 to about 25 weight % of a filler.

In certain embodiments, the pharmaceutical composition comprises: (a) 25 to 60 weight % of a solid dispersion of tucatinib; (b) 1 to 10 weight % of a disintegrant; (c) 15 to 25 weight % of an osmogen; (d) 0.1 to 3 weight % of a glidant; (e) 0.1 to 3 weight % of a lubricant; and (f) 10 to 25 weight % of a filler.

Certain embodiments provide a pharmaceutical composition comprising: (a) about 40 to about 60 weight % of a solid dispersion of tucatinib; (b) about 1 to about 10 weight % of a disintegrant; (c) about 15 to about 25 weight % of an osmogen; (d) about 0.1 to about 3 weight % of a glidant; (e) about 0.1 to about 3 weight % of a lubricant; and (f) about 10 to about 25 weight % of a filler.

In certain embodiments, the pharmaceutical composition comprises: (a) 40 to 60 weight % of a solid dispersion of tucatinib; (b) 1 to 10 weight % of a disintegrant; (c) 15 to 25 weight % of an osmogen; (d) 0.1 to 3 weight % of a glidant; (e) 0.1 to 3 weight % of a lubricant; and (f) 10 to 25 weight % of a filler.

In certain embodiments, the osmogen is selected from NaCl and KCl, and mixtures thereof.

In certain embodiments, the lubricant is magnesium stearate.

In certain embodiments, the glidant is colloidal silicon dioxide.

In certain embodiments, the binder/diluent is microcrystalline cellulose. In certain embodiments, the binder/diluent acts as both a binder and a diluent.

In certain embodiments, the binder is microcrystalline cellulose.

In certain embodiments, the diluent is microcrystalline cellulose.

In certain embodiments, the filler is lactose.

In certain embodiments, the disintegrant is selected from crospovidone and sodium bicarbonate (NaHCO₃), and mixtures thereof. In certain embodiments, the disintegrant is selected from crospovidone and sodium bicarbonate. In certain embodiments, the disintegrant is sodium bicarbonate. In certain embodiments, the disintegrant is crospovidone.

In certain embodiments, the composition contains sodium bicarbonate. tucatinib may slowly degrade, through hydrolysis or other means, to a carbamate impurity:

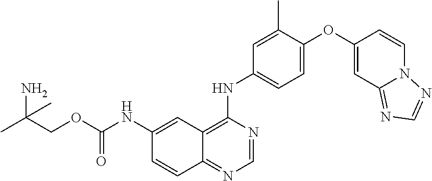 [SMILES: Cc1cc(Nc2ncnc3ccc(NC(=O)OCC(C)(C)N)cc23)ccc1Oc1ccn2ncnc2c1]

Sodium bicarbonate helps to slow the degradation to the carbamate impurity. Sodium bicarbonate also helps to provide consistent tablet disintegration when the tablets are exposed to different humidities.

Certain embodiments provide a pharmaceutical composition comprising: (a) tucatinib; and (b) sodium bicarbonate.

Certain embodiments provide a pharmaceutical composition comprising: (a) about 1 to about 70 weight % of a solid dispersion of tucatinib; and (b) about 0.1 to about 30 weight % sodium bicarbonate.

In certain embodiments, the pharmaceutical composition comprises: (a) 1 to 70 weight % of a solid dispersion of tucatinib; and (b) 0.1 to 30 weight % sodium bicarbonate.

Certain embodiments provide a pharmaceutical composition comprising: (a) about 1 to about 70 weight % of a solid dispersion of tucatinib; (b) about 0.1 to about 30 weight % sodium bicarbonate; and (c) the remaining weight is other pharmaceutically acceptable excipients and carriers.

In certain embodiments, the pharmaceutical composition comprises: (a) 1 to 70 weight % of a solid dispersion of tucatinib; (b) 0.1 to 30 weight % sodium bicarbonate; and (c) the remaining weight is other pharmaceutically acceptable excipients and carriers.

Certain embodiments provide a pharmaceutical composition comprising: (a) about 25 to about 60 weight % of a solid dispersion of tucatinib; and (b) about 1 to about 15 weight % of sodium bicarbonate.

In certain embodiments, the pharmaceutical composition comprises: (a) 25 to 60 weight % of a solid dispersion of tucatinib; and (b) 1 to 15 weight % of sodium bicarbonate.

Certain embodiments provide a pharmaceutical composition comprising: (a) about 25 to about 60 weight % of a solid dispersion of tucatinib; (b) about 1 to about 15 weight % of sodium bicarbonate; and (c) the remaining weight is other pharmaceutically acceptable excipients and carriers.

In certain embodiments, the pharmaceutical composition comprises: (a) 25 to 60 weight % of a solid dispersion of tucatinib; (b) 1 to 15 weight % of sodium bicarbonate; and (c) the remaining weight is other pharmaceutically acceptable excipients and carriers.

Certain embodiments provide a pharmaceutical composition comprising: (a) about 40 to about 60 weight % of a solid dispersion of tucatinib; and (b) about 1 to about 15 weight % of sodium bicarbonate.

In certain embodiments, the pharmaceutical composition comprises: (a) 40 to 60 weight % of a solid dispersion of tucatinib; and (b) 1 to 15 weight % of sodium bicarbonate.

Certain embodiments provide a pharmaceutical composition comprising: (a) about 40 to about 60 weight % of a solid dispersion of tucatinib; (b) about 1 to about 15 weight % of sodium bicarbonate; (c) the remaining weight is other pharmaceutically acceptable excipients and carriers.

In certain embodiments, the pharmaceutical composition comprises: (a) 40 to 60 weight % of a solid dispersion of tucatinib; (b) 1 to 15 weight % of sodium bicarbonate; (c) the remaining weight is other pharmaceutically acceptable excipients and carriers.

Certain embodiments provide a pharmaceutical composition comprising: (a) about 40 to about 60 weight % of a solid dispersion of tucatinib; (b) about 5 to about 15 weight % of a disintegrant which is selected from the group of crospovidone, sodium bicarbonate (NaHCO₃), and mixtures thereof; (c) about 15 to about 25 weight % of an osmogen which is selected from the group consisting of NaCl, KCl, and mixtures thereof, (d) about 0.1 to about 3 weight % of a glidant which is colloidal silicon dioxide; (e) about 0.1 to about 3 weight % of a lubricant which is magnesium stearate; and (f) about 10 to about 25 weight % of a binder/diluent which is microcrystalline cellulose.

In certain embodiments, the pharmaceutical composition comprises: (a) 40 to 60 weight % of a solid dispersion of tucatinib; (b) 5 to 15 weight % of a disintegrant which is selected from the group of crospovidone, sodium bicarbonate (NaHCO₃), and mixtures thereof, (c) 15 to 25 weight % of an osmogen which is selected from the group consisting of NaCl, KCl, and mixtures thereof; (d) 0.1 to 3 weight % of a glidant which is colloidal silicon dioxide; (e) 0.1 to 3 weight % of a lubricant which is magnesium stearate; and (f) 10 to 25 weight % of a binder/diluent which is microcrystalline cellulose.

Certain embodiments provide a pharmaceutical composition comprising: (a) about 40 to about 60 weight % of a solid dispersion of tucatinib; (b) about 1 to about 10 weight % of a disintegrant which is selected from the group of crospovidone, sodium bicarbonate (NaHCO₃), and mixtures thereof; (c) about 15 to about 25 weight % of an osmogen which is selected from the group consisting of NaCl, KCl, and mixtures thereof, (d) about 0.1 to about 3 weight % of a glidant which is colloidal silicon dioxide; (e) about 0.1 to about 3 weight % of a lubricant which is magnesium stearate; and (f) about 10 to about 25 weight % of a filler which is lactose.

In certain embodiments, the pharmaceutical composition comprises: (a) 40 to 60 weight % of a solid dispersion of tucatinib; (b) 1 to 10 weight % of a disintegrant which is selected from the group of crospovidone, sodium bicarbonate (NaHC₃), and mixtures thereof, (c) 15 to 25 weight % of an osmogen which is selected from the group consisting of NaCl, KCl, and mixtures thereof; (d) 0.1 to 3 weight % of a glidant which is colloidal silicon dioxide; (e) 0.1 to 3 weight % of a lubricant which is magnesium stearate; and (f) 10 to 25 weight % of a filler which is lactose.

In certain embodiments, the pharmaceutical composition is selected from the group consisting of:

| Function | Ingredient | % of Blend |
|---|---|---|
| API | Solid dispersion of tucatinib | about 50 |
| Disintegrant | Crospovidone - Polyplasdone ® | about 6 |
| Osmogen | NaCl | about 5 |
| Osmogen | KCl | about 5 |
| Glidant | Colloidal Silicon Dioxide | about 0.5 |
| Lubricant | Magnesium Stearate | about 0.25 |

Extragranular

| | | |
|---|---|---|
| Binder/Diluent | Microcrystalline cellulose - Avicel ® | about 19.25 |
| Osmogen | NaCl | about 4.625 |
| Osmogen | KCl | about 4.625 |
| Disintegrant | Polyplasdone | about 4 |
| Glidant | Colloidal Silicon Dioxide | about 0.5 |
| Lubricant | Magnesium Stearate | about 0.25 |

| Function | Ingredient | % of Blend |
|---|---|---|
| API | Solid dispersion of tucatinib | about 50 |
| Disintegrant | Crospovidone - Polyplasdone ® | about 6 |
| Disintegrant | NaHCO₃ | about 3 |
| Osmogen | NaCl | about 5 |
| Osmogen | KCl | about 5 |
| Glidant | Colloidal Silicon Dioxide | about 0.5 |
| Lubricant | Magnesium Stearate | about 0.25 |

Extragranular

| | | |
|---|---|---|
| Binder/Diluent | Microcrystalline cellulose - Avicel ® | about 16.25 |
| Osmogen | NaCl | about 4.625 |
| Osmogen | KCl | about 4.625 |
| Disintegrant | Polyplasdone | about 4 |
| Glidant | Colloidal Silicon Dioxide | about 0.5 |
| Lubricant | Magnesium Stearate | about 0.25 |

| Function | Ingredient | % of Blend |
|---|---|---|
| API | Solid dispersion of tucatinib | about 50 |
| Disintegrant | Crospovidone - Polyplasdone ® | about 6 |
| Osmogen | NaCl | about 10.625 |
| Osmogen | KCl | about 10.625 |
| Filler | Lactose | about 21.25 |
| Glidant | Colloidal Silicon Dioxide | about 0.5 |
| Lubricant | Magnesium Stearate | aabout 0.25 |

Extragranular

| | | |
|---|---|---|
| Glidant | Colloidal Silicon Dioxide | about 0.5 |
| Lubricant | Magnesium Stearate | about 0.25 |

In certain embodiments, the pharmaceutical composition is selected from the group consisting of:

| Function | Ingredient | % of Blend |
|---|---|---|
| API | Solid dispersion of tucatinib | 50 |
| Disintegrant | Crospovidone - Polyplasdone ® | 6 |
| Osmogen | NaCl | 5 |
| Osmogen | KCl | 5 |
| Glidant | Colloidal Silicon Dioxide | 0.5 |
| Lubricant | Magnesium Stearate | 0.25 |

Extragranular

| | | |
|---|---|---|
| Binder/Diluent | Microcrystalline cellulose - Avicel ® | 19.25 |
| Osmogen | NaCl | 4.625 |
| Osmogen | KCl | 4.625 |
| Disintegrant | Polyplasdone | 4 |
| Glidant | Colloidal Silicon Dioxide | 0.5 |
| Lubricant | Magnesium Stearate | 0.25 |

| Function | Ingredient | % of Blend |
|---|---|---|
| API | Solid dispersion of tucatinib | 50 |
| Disintegrant | Crospovidone - Polyplasdone ® | 6 |
| Disintegrant | NaHCO₃ | 3 |
| Osmogen | NaCl | 5 |
| Osmogen | KCl | 5 |
| Glidant | Colloidal Silicon Dioxide | 0.5 |
| Lubricant | Magnesium Stearate | 0.25 |

Extragranular

| | | |
|---|---|---|
| Binder/Diluent | Microcrystalline cellulose - Avicel ® | 16.25 |
| Osmogen | NaCl | 4.625 |
| Osmogen | KCl | 4.625 |
| Disintegrant | Polyplasdone | 4 |
| Glidant | Colloidal Silicon Dioxide | 0.5 |
| Lubricant | Magnesium Stearate | 0.25 |

| Function | Ingredient | % of Blend |
|---|---|---|
| API | Solid dispersion of tucatinib | 50 |
| Disintegrant | Crospovidone - Polyplasdone ® | 6 |
| Osmogen | NaCl | 10.625 |
| Osmogen | KCl | 10.625 |
| Filler | Lactose | 21.25 |
| Glidant | Colloidal Silicon Dioxide | 0.5 |
| Lubricant | Magnesium Stearate | 0.25 |

Extragranular

| | | |
|---|---|---|
| Glidant | Colloidal Silicon Dioxide | 0.5 |
| Lubricant | Magnesium Stearate | 0.25 |

The pharmaceutical composition preferably contains a therapeutically effective amount of tucatinib. However, in some embodiments, each individual dose contains a portion of a therapeutically effective amount of tucatinib, such that multiple doses of the composition may be required (for example, two or more tablets are required for a therapeutically effective amount). Thus, in this application when it states that the pharmaceutical composition contains a therapeutically effective amount it means that the composition may be one dose (for example, one tablet) or multiple doses (for example, two tablets). In certain embodiments, the pharmaceutical composition contains between 1 and 500 mg of tucatinib.

In certain embodiments, the pharmaceutical composition contains between about 25 and about 400 mg of tucatinib. In certain embodiments, the pharmaceutical composition contains between 25 and 400 mg of tucatinib.

In certain embodiments, the pharmaceutical composition contains between about 25 and about 100 mg (e.g., about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg) of tucatinib. In certain embodiments, the pharmaceutical composition contains between 25 and 100 mg (e.g., 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, and 100 mg) of tucatinib. In certain embodiments, the pharmaceutical composition contains between about 25 and about 75 mg of tucatinib. In certain embodiments, the pharmaceutical composition contains between 25 and 75 mg of tucatinib. In certain embodiments, the pharmaceutical composition contains about 50 mg of tucatinib. In certain particular embodiments, the pharmaceutical composition contains 50 mg of tucatinib. In certain of the foregoing embodiments, the pharmaceutical composition is formulated as a tablet. As a non-limiting example, the pharmaceutical composition is formulated as a tablet and contains 50 mg of tucatinib.

In certain embodiments, the pharmaceutical composition contains between about 100 and about 300 mg (e.g., about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg) of tucatinib. In certain embodiments, the pharmaceutical composition contains between 100 and 300 mg (e.g., 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, 300 mg) of tucatinib. In certain embodiments, the pharmaceutical composition contains between about 100 and about 200 mg of tucatinib. In certain embodiments, the pharmaceutical composition contains between 100 and 200 mg of tucatinib. In certain embodiments, the pharmaceutical composition contains between about 125 and about 175 mg of tucatinib. In certain embodiments, the pharmaceutical composition contains between 125 and 175 mg of tucatinib. In certain embodiments, the pharmaceutical composition contains about 150 mg of tucatinib. In certain particular embodiments, the pharmaceutical composition contains 150 mg of tucatinib. In certain of the foregoing embodiments, the pharmaceutical composition is formulated as a tablet. As a non-limiting example, the pharmaceutical composition is formulated as a tablet and contains 150 mg of tucatinib.

The pharmaceutical compositions described herein may be administered by any convenient route appropriate to the condition to be treated. Suitable routes include oral, parenteral (including subcutaneous, intramuscular, intravenous, intraarterial, intradermal, intrathecal and epidural), transdermal, rectal, nasal, topical (including buccal and sublingual), ocular, vaginal, intraperitoneal, intrapulmonary and intranasal. If parenteral administration is desired, the compositions will be sterile and in a solution or suspension form suitable for injection or infusion.

The compounds may be administered in any convenient administrative form, e.g., tablets, powders, capsules, dispersions, suspensions, syrups, sprays, suppositories, gels, emulsions, patches, etc.

The pharmaceutical compositions described herein are typically administered orally. The pharmaceutical compositions described herein are typically administered as a tablet, caplet, hard or soft gelatin capsule, pill, granules or a suspension.

Additional examples of pharmaceutical compositions of tucatinib and methods of preparation thereof are described in U.S. Pat. No. 9,457,093, which is incorporated by reference herein in its entirety.

The pharmaceutical compositions described herein may comprise one or more polymorphs of tucatinib. Exemplary polymorphs of tucatinib and methods of preparation thereof are described in U.S. Pat. No. 9,168,254, which is incorporated by reference herein in its entirety.

In some embodiments, the pharmaceutical composition comprises amorphous tucatinib. In certain embodiments, tucatinib in the pharmaceutical composition is substantially amorphous (e.g., at least 80%, at least 85%, at least 90%, or at least 95% amorphous).

In some embodiments, the pharmaceutical composition comprises a crystalline polymorph of tucatinib. In certain embodiments, tucatinib in the pharmaceutical composition is substantially crystalline (e.g., at least 80%, at least 85%, at least 90%, or at least 95% crystalline).

In certain embodiments, the pharmaceutical composition comprises polymorph Form A of tucatinib as described in U.S. Pat. No. 9,168,254. In certain embodiments, tucatinib in the pharmaceutical composition is substantially in Form A (e.g., at least 80%, at least 85%, at least 90%, or at least 95% Form A).

In certain embodiments, the pharmaceutical composition comprises polymorph Form B of tucatinib as described in U.S. Pat. No. 9,168,254. In certain embodiments, tucatinib in the pharmaceutical composition is substantially in Form B (e.g., at least 80%, at least 85%, at least 90%, or at least 95% Form B).

In certain embodiments, the pharmaceutical composition comprises polymorph Form C of tucatinib as described in U.S. Pat. No. 9,168,254. In certain embodiments, tucatinib in the pharmaceutical composition is substantially in Form C (e.g., at least 80%, at least 85%, at least 90%, or at least 95% Form C).

In certain embodiments, the pharmaceutical composition comprises polymorph Form D of tucatinib as described in U.S. Pat. No. 9,168,254. In certain embodiments, tucatinib in the pharmaceutical composition is substantially in Form D (e.g., at least 80%, at least 85%, at least 90%, or at least 95% Form D).

In certain embodiments, the pharmaceutical composition comprises polymorph Form E of tucatinib as described in U.S. Pat. No. 9,168,254. In certain embodiments, tucatinib in the pharmaceutical composition is substantially in Form E (e.g., at least 80%, at least 85%, at least 90%, or at least 95% Form E).

In certain embodiments, the pharmaceutical composition comprises polymorph Form F of tucatinib as described in U.S. Pat. No. 9,168,254. In certain embodiments, tucatinib in the pharmaceutical composition is substantially in Form F (e.g., at least 80%, at least 85%, at least 90%, or at least 95% Form F).

In certain embodiments, the pharmaceutical composition comprises polymorph Form G of tucatinib as described in U.S. Pat. No. 9,168,254. In certain embodiments, tucatinib in the pharmaceutical composition is substantially in Form G (e.g., at least 80%, at least 85%, at least 90%, or at least 95% Form G).

In certain embodiments, the pharmaceutical composition comprises polymorph Form H of tucatinib as described in U.S. Pat. No. 9,168,254. In certain embodiments, tucatinib in the pharmaceutical composition is substantially in Form H (e.g., at least 80%, at least 85%, at least 90%, or at least 95% Form H).

In certain embodiments, the pharmaceutical composition comprises polymorph Form I of tucatinib as described in U.S. Pat. No. 9,168,254. In certain embodiments, tucatinib in the pharmaceutical composition is substantially in Form I (e.g., at least 80%, at least 85%, at least 90%, or at least 95% Form I).

In certain embodiments, the pharmaceutical composition comprises polymorph Form J of tucatinib as described in U.S. Pat. No. 9,168,254. In certain embodiments, tucatinib in the pharmaceutical composition is substantially in Form J (e.g., at least 80%, at least 85%, at least 90%, or at least 95% Form J).

In certain embodiments, the pharmaceutical composition comprises polymorph Form K of tucatinib as described in U.S. Pat. No. 9,168,254. In certain embodiments, tucatinib in the pharmaceutical composition is substantially in Form K (e.g., at least 80%, at least 85%, at least 90%, or at least 95% Form K).

In certain embodiments, the pharmaceutical composition comprises polymorph Form L of tucatinib as described in U.S. Pat. No. 9,168,254. In certain embodiments, tucatinib in the pharmaceutical composition is substantially in Form L (e.g., at least 80%, at least 85%, at least 90%, or at least 95% Form L).

In certain embodiments, the pharmaceutical composition comprises polymorph Form M of tucatinib as described in U.S. Pat. No. 9,168,254. In certain embodiments, tucatinib in the pharmaceutical composition is substantially in Form M (e.g., at least 80%, at least 85%, at least 90%, or at least 95% Form M).

In certain embodiments, the pharmaceutical composition comprises polymorph Form N of tucatinib as described in U.S. Pat. No. 9,168,254. In certain embodiments, tucatinib in the pharmaceutical composition is substantially in Form N (e.g., at least 80%, at least 85%, at least 90%, or at least 95% Form N).

In certain embodiments, the pharmaceutical composition comprises polymorph Form O of tucatinib as described in U.S. Pat. No. 9,168,254. In certain embodiments, tucatinib in the pharmaceutical composition is substantially in Form O (e.g., at least 80%, at least 85%, at least 90%, or at least 95% Form O).

In certain embodiments, the pharmaceutical composition comprises polymorph Form P of tucatinib as described in U.S. Pat. No. 9,168,254. In certain embodiments, tucatinib in the pharmaceutical composition is substantially in Form P (e.g., at least 80%, at least 85%, at least 90%, or at least 95% Form P).

### H. Articles of Manufacture and Kits

In another aspect, the present disclosure provides an article of manufacture or kit for treating or ameliorating the effects of a HER2 positive cancer in a subject, the kit comprising tucatinib, trastuzumab, a taxane, and a VEGFR-2 antagonist (e.g., tucatinib, trastuzumab, paclitaxel, and ramucirumab).

The articles of manufacture or kits are suitable for treating or ameliorating the effects of HER2 positive and/or metastatic cancer in a subject. In some embodiments, the cancer is an advanced cancer. In some other embodiments, the cancer is a drug-resistant cancer. In some instances, the cancer is a multidrug-resistant cancer.

Materials and reagents to carry out the various methods of the present disclosure can be provided in articles of manufacture or kits to facilitate execution of the methods. As used herein, the term “kit” includes a combination of articles that facilitates a process, assay, analysis, or manipulation. In particular, kits of the present disclosure find utility in a wide range of applications including, for example, diagnostics, prognostics, therapy, and the like.

Articles of manufacture or kits can contain chemical reagents as well as other components. In addition, the articles of manufacture or kits of the present disclosure can include, without limitation, instructions to the user, apparatus and reagents for administering combinations of tucatinib, trastuzumab, a taxane, and a VEGFR-2 antagonist (e.g., tucatinib, trastuzumab, paclitaxel, and ramucirumab) or pharmaceutical compositions thereof, sample tubes, holders, trays, racks, dishes, plates, solutions, buffers, or other chemical reagents. Articles of manufacture or kits of the present disclosure can also be packaged for convenient storage and safe shipping, for example, in a box having a lid.

### III. Exemplary Embodiments

It is understood that the examples and embodiments described herein are for illustrative purposes only and that various modifications or changes in light thereof will be suggested to persons skilled in the art and are to be included within the spirit and purview of this application and scope of the appended claims. All publications, patents, patent applications, and sequence accession numbers cited herein are hereby incorporated by reference in their entirety for all purposes.

The disclosure will be more fully understood by reference to the following examples. They should not, however, be construed as limiting the scope of the disclosure. It is understood that the examples and embodiments described herein are for illustrative purposes only and that various modifications or changes in light thereof will be suggested to persons skilled in the art and are to be included within the spirit and purview of this application and scope of the appended claims.

### Example 1: Phase 2/3, Study of Tucatinib in Combination with Trastuzumab, a Taxane, and a VEGFR-2 Antagonist in Subjects with Previously Treated, Locally-Advanced Unresectable or Metastatic HER2+ Gastric or Gastroesophageal Junction Adenocarcinoma (GEC)

Study Objectives

The Phase 2 portion of this study will determine the recommended dose of paclitaxel when administered in combination with tucatinib, trastuzumab, and ramucirumab, evaluate the safety and tolerability of tucatinib in combination with trastuzumab, ramucirumab, and paclitaxel, and evaluate the activity, and pharmacokinetics (PK) of the regimen in subjects with locally-advanced unresectable or metastatic HER2+ GEC who have received prior treatment with a HER2-directed antibody in the locally-advanced unresectable or metastatic disease setting. Specific objectives and corresponding endpoints for the study are summarized below (

Table 1).

TABLE 1
Objectives and corresponding endpoints (Phase 2)

| | Corresponding Endpoints |
|---|---|
| Primary Objectives | |
| To determine the recommended dose | Frequency of dose-limiting toxicities (DLT) during |
| of paclitaxel when administered in | the first cycle of treatment with tucatinib, |
| combination with tucatinib, | trastuzumab, ramucirumab, and paclitaxel |
| trastuzumab, and ramucirumab | |
| To evaluate the safety and tolerability | Type, incidence, severity, seriousness, and relatedness |
| of tucatinib in combination with | of adverse events (AEs) and laboratory |
| trastuzumab, ramucirumab, and | abnormalities |
| paclitaxel | Vital signs and other relevant safety variables |
| | Frequency of dose holds, dose reductions, and |
| | discontinuations of tucatinib, paclitaxel, |
| | trastuzumab, and ramucirumab |
| Secondary Objectives | |
| To evaluate the preliminary activity | ORR (complete response [CR] or partial response |
| of tucatinib in combination with | [PR]) per Response evaluation criteria in solid |
| trastuzumab, ramucirumab, and | tumors (RECIST) version 1.1 according to |
| paclitaxel in subjects with | investigator assessment |
| previously treated locally-advanced | Confirmed ORR per RECIST version 1.1 according to |
| unresectable or metastatic GEC that | investigator assessment |
| is HER2+ according to blood-based | PFS per RECIST version 1.1 according to investigator |
| next generation sequencing (NGS) | assessment |
| assay of circulating tumor DNA | Duration of response (DOR; CR or PR) per RECIST |
| (ctDNA) | version 1.1 according to investigator assessment |
| | Disease control rate (DCR; CR or PR or stable |
| | disease/non-CR, non-progressive disease as best |
| | objective response) per RECIST version 1.1 |
| | according to investigator assessment |
| To evaluate the PK of tucatinib and | The PK parameters to be calculated for tucatinib, |
| the tucatinib metabolite ONT-993 | paclitaxel, and their respective metabolites (if |
| To evaluate the PK of paclitaxel and | applicable) may include but are not limited to: area |
| its metabolites in the presence and | under the plasma concentration-time curve (AUC) to |
| absence of tucatinib | the time of the last quantifiable concentration |
| | (AUCₗₐₛₜ), maximum observed concentration (Cₘₐₓ), |
| | time of Cₘₐₓ (Tₘₐₓ), trough concentration (C_{trough}), |
| | and metabolic ratio based on AUC (MR_{AUC}) |
| Exploratory Objectives | |
| To evaluate the preliminary activity | ORR per RECIST version 1.1 according to |
| of tucatinib in combination with | investigator assessment |
| trastuzumab, ramucirumab, and | Confirmed ORR per RECIST version 1.1 according |
| paclitaxel in subjects with disease | to investigator assessment |
| that is HER2-negative according to | PFS per RECIST version 1.1 according to |
| blood-based NGS assay but HER2+ | investigator assessment |
| according to tissue biopsy | Duration of response per RECIST version 1.1 |
| | according to investigator assessment |
| | Disease control rate per RECIST version 1.1 |
| | according to investigator assessment |
| To evaluate the correlation between | Frequency of HER2 alterations according to blood- |
| HER2 alterations as detected by | based NGS assay and according to assays on biopsy |
| blood-based NGS assays and | samples obtained after progression on the most |
| standard tissue-based assays | recent line of therapy |
| To explore correlations between | Potential biomarkers of response, resistance, or |
| blood-based biomarkers and | toxicity may be evaluated in blood |
| clinical outcomes | |
| To evaluate the PK of tucatinib and | The PK parameters of tucatinib and ONT-993 in |
| ONT-993 in subjects with and | subjects with and without gastrectomies may include |
| without gastrectomy | but are not limited to: AUCₗₐₛₜ, Cₘₐₓ, Tₘₐₓ, C_{trough}, |
| | and MR_{AUC} |

The Phase 3 portion will compare the efficacy, safety, patient-reported outcomes (PRO), and health care resource utilization (HCRU) of tucatinib and trastuzumab versus placebo in combination with ramucirumab and paclitaxel. It will also evaluate the activity of tucatinib combined with ramucirumab and paclitaxel. Specific objectives and corresponding endpoints for the study are summarized below (Table 2).

TABLE 2
Objectives and corresponding endpoints (Phase 3)

| | Corresponding Endpoints |
|---|---|
| Primary Objective | |
| To compare the efficacy of | Dual primary endpoints |
| tucatinib and trastuzumab | OS |
| versus placebo, in combination | PFS per RECIST version 1.1 according to investigator |
| with ramucirumab and | assessment |
| paclitaxel in subjects with | Key secondary endpoints |
| previously treated, locally- | Confirmed ORR per RECIST version 1.1 according to |
| advanced unresectable or | investigator assessment |
| metastatic HER2+ GEC | Other secondary endpoints |
| | PFS per RECIST version 1.1 according to blinded |
| | independent central review (BICR) assessment |
| | Confirmed ORR per RECIST version 1.1 according to BICR |
| | assessment |
| | ORR per RECIST version 1.1 according to investigator |
| | assessment |
| | ORR per RECIST version 1.1 according to BICR |
| | assessment |
| | DOR per RECIST version 1.1 according to investigator |
| | assessment |
| | DOR per RECIST version 1.1 according to BICR assessment |
| | DCR per RECIST version 1.1 according to investigator |
| | assessment |
| | DCR per RECIST version 1.1 according to BICR assessment |
| Secondary Objectives | |
| To evaluate the safety and | Type, incidence, severity, seriousness, and relatedness of |
| tolerability of tucatinib in | AEs and laboratory abnormalities |
| combination with | Vital signs and other relevant safety variables |
| trastuzumab, ramucirumab, | Frequency of dose holds, dose reductions, and |
| and paclitaxel | discontinuations of tucatinib, trastuzumab, ramucirumab, |
| | and paclitaxel |
| To evaluate the anti-tumor | Secondary Endpoints |
| activity of tucatinib in | Confirmed ORR per RECIST version 1.1 according to |
| combination with | investigator assessment |
| ramucirumab and paclitaxel | DOR per RECIST version 1.1 according to investigator |
| | assessment |
| | Exploratory Endpoints |
| | OS |
| | PFS per RECIST version 1.1 according to investigator |
| | assessment |
| | PFS per RECIST version 1.1 according to BICR assessment |
| | Confirmed ORR per RECIST version 1.1 according to BICR |
| | assessment |
| | ORR per RECIST version 1.1 according to investigator |
| | assessment |
| | ORR per RECIST version 1.1 according to BUCR |
| | assessment |
| | DOR per RECIST version 1.1 according to investigator |
| | assessment |
| | DCR per RECIST version 1.1 according to investigator |
| | assessment |
| | DCR per RECIST version 1.1 according to BICR assessment |
| To assess PROs by treatment | Time to deterioration of GEC symptoms as assessed by the |
| arm | European Organisation for Research and Treatment of |
| | Cancer (EORTC) quality of life questionnaire (QLQ)-C30 |
| | and EORTC QLQ-OG25 questionnaires |
| | Change from baseline in health-related quality of life |
| | (HRQoL) |
| | Utility index values as assessed by the EQ-5D-5L |
| Exploratory Objectives | |
| To evaluate the safety and | Type, incidence, severity, seriousness, and relatedness of |
| tolerability of tucatinib in | AEs and laboratory abnormalities |
| combination with | Vital signs and other relevant safety variables |
| ramucirumab, and paclitaxel | Frequency of dose holds, dose reductions, and |
| | discontinuations of tucatinib, ramucirumab, and paclitaxel |
| To evaluate the PK of tucatinib | The PK parameter to be calculated for tucatinib (if |
| | applicable) includes: C_{trough} |
| To explore correlations | Potential biomarkers of response, resistance, or toxicity may |
| between blood-based | be evaluated in blood |
| biomarkers and clinical | |
| outcomes | |
| To assess HCRU by treatment | Cumulative incidence of healthcare resource utilization, |
| arm | including length of stay, hospitalizations, and emergency |
| | department visits |

### Number of Planned Subjects

Phase 2: Approximately 66 subjects will be enrolled and treated
Phase 3: Approximately 500 subjects will be randomized to 3 arms.

### Investigational Plan

### Summary of Study Design

This is an international, multicenter Phase 2/3 study in subjects with locally-advanced unresectable or metastatic HER2+ GEC who have received prior treatment with a HER2 directed antibody, and have received 1 prior line of therapy in the advanced disease setting.

The study is composed of the following parts:

### Open-Label Phase 2 Portion:

Paclitaxel dose optimization stage: this single-arm stage will determine the recommended dose of paclitaxel when combined with tucatinib, trastuzumab, and ramucirumab

Dose expansion stage: this 2-cohort stage will enroll subjects to further evaluate the safety and activity of the regimen in a total of approximately 30 response evaluable subjects in each cohort, once the recommended paclitaxel dose is determined.

Randomized, double-blind, placebo-controlled Phase 3 portion: will compare the efficacy and evaluate the safety of tucatinib and trastuzumab versus placebo, in combination with ramucirumab and paclitaxel, and evaluate the efficacy and safety of tucatinib combined with ramucirumab and paclitaxel.

Dose reductions of tucatinib (or placebo), ramucirumab, and paclitaxel will be allowed. Dose reductions of trastuzumab (or placebo) will not be allowed; if trastuzumab cannot be restarted after being held for an AE, it must be discontinued. If any study drug is discontinued, the subject can continue to receive study treatment with the remaining agents. Study treatment (defined as the administration of any of the 4 study drugs, without initiation of new anti-cancer treatment) will continue until unacceptable toxicity, disease progression, withdrawal of consent, death, or study closure. Disease response and progression will be assessed using RECIST version 1.1.

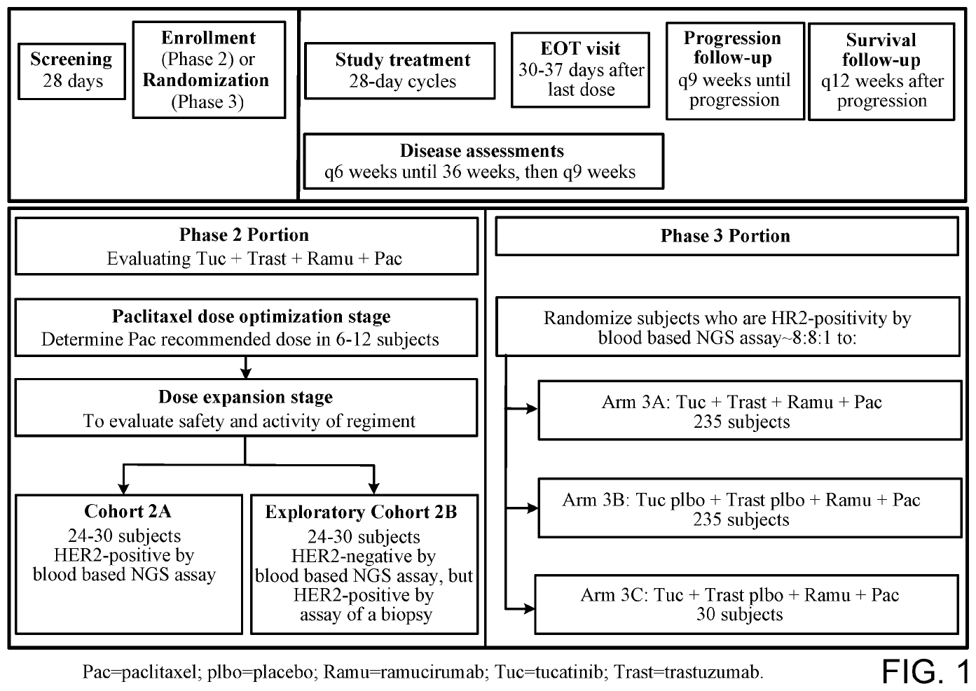

While on study treatment, radiographic disease evaluations will be done every 6 weeks for the first 36 weeks, and every 9 weeks thereafter, irrespective of dose holds or interruptions (FIG. 1). All efforts should be made to continue treatment until unequivocal evidence of radiologic progression occurs according to RECIST version 1.1. If study treatment is discontinued before documentation of disease progression, radiographic evaluations will be performed at least every 9 weeks until the occurrence of progression, withdrawal of consent, or study closure. After occurrence of disease progression, subjects will continue to be followed for survival every 12 weeks, until death, consent withdrawal, or study closure.

### Phase 2—Safety Lead-In

### Phase 2

### Paclitaxel Dose Optimization Stage

In the paclitaxel dose optimization stage of the study, the initial paclitaxel dose will be 60 mg/m² intravenous (IV) on Days 1, 8, and 15 of each 28-day cycle, in combination with tucatinib 300 mg orally (PO) twice daily (BID), trastuzumab (6 mg/kg IV loading dose on cycle 1 day 1, 4 mg/kg IV on cycle 1 day 15 and days 1 and 15 of each cycle thereafter), and ramucirumab (8 mg/kg IV on days 1 and 15).

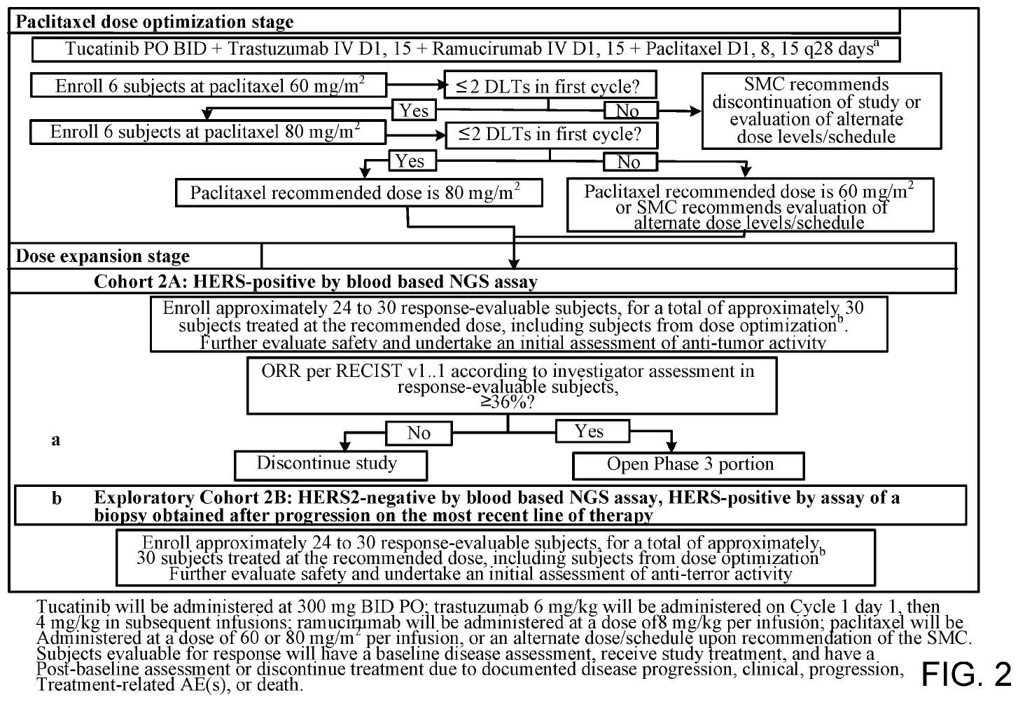

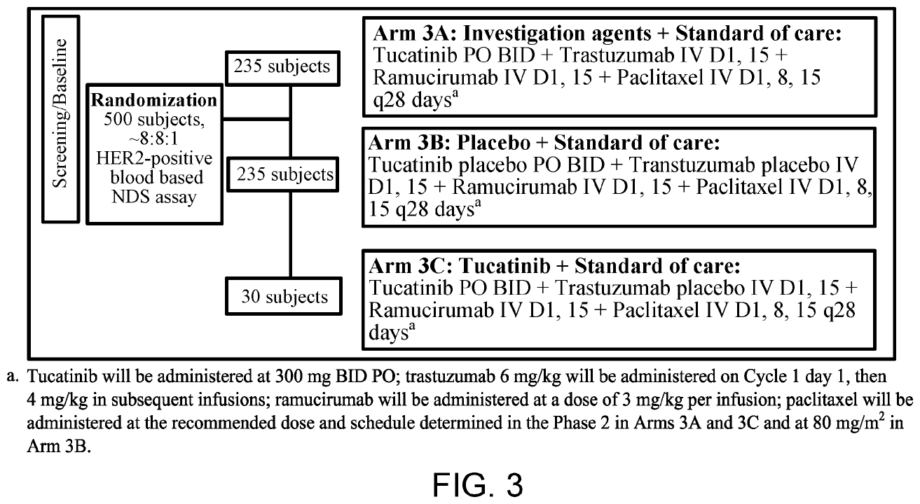

Six subjects will initially be enrolled and treated at paclitaxel 60 mg/m² (FIG. 2). Subjects can have centrally confirmed HER2+ disease according to a blood-based NGS assay of ctDNA done at screening or IHC/ISH assay of tissue biopsies obtained after progression on the most recent line of systemic therapy. Once 6 subjects are evaluable for DLT, enrollment will be paused and the Safety Monitoring Committee (SMC) will undertake an evaluation of safety and PK. If necessary, additional subjects will be enrolled to replace subjects inevaluable for DLT. If >2 DLTs are observed in the 6 subjects receiving 60 mg/m², the evaluation of the regimen will be halted, or an alternative dose level/schedule may be recommended by the SMC. If ≤2 DLT are observed, the paclitaxel dose will be escalated to 80 mg/m² and evaluated in a further 6 subjects. If >2 DLTs are observed at the 80 mg/m² dose level, the 60 mg/m² paclitaxel dose will be declared the recommended dose, and evaluation of the regimen will continue in the Dose Expansion stage. Otherwise, 80 mg/m² will be the recommended dose. The SMC may also recommend the inclusion of additional subjects in any dose level or the evaluation of an alternative dose level/schedule, if necessary. The SMC will continuously monitor subjects for AEs, deaths, other serious adverse events (SAEs), dose modifications, and laboratory abnormalities, with a specific focus on DLTs.

Dose Expansion Stage

Following the paclitaxel dose optimization stage, two cohorts will be opened to enroll further subjects. Cohort 2A will enroll subjects with HER2+ disease as determined by HER2 amplification in a blood-based NGS assay of ctDNA performed at a central laboratory at screening. The exploratory Cohort 2B will enroll subjects whose disease did not show HER2 amplification in the blood based NGS assay, but did show centrally confirmed HER2 overexpression/amplification according to the package insert of FDA approved tests for immunohistochemistry (IHC) and in situ hybridization (ISH) in a biopsy obtained after progression on the most recent line of systemic therapy.

For each type of HER2 positivity evaluated (blood-based NGS assay or IHC/ISH assay of a tissue biopsy), approximately 30 response-evaluable subjects who have been treated at the recommended dose in either the dose optimization stage or the dose expansion stage will be enrolled. Consequently, approximately 24 to 30 response-evaluable subjects will be enrolled in each cohort during the dose expansion stage, in order to further evaluate the safety of the study regimen and undertake an initial assessment of anti-tumor activity. Subjects who are not evaluable for response will be replaced.

The SMC will evaluate the safety of the study regimen throughout the remainder of the Phase 2, in the 2 cohorts. At least 6 subjects with a history of prior gastrectomy (without maintenance of the pylorus) will be enrolled in either cohort to evaluate the PK of tucatinib and ONT-993 in this population; alternative dose levels/schedules may be explored depending on the PK of tucatinib in subjects with gastrectomy.

A formal efficacy analysis will be undertaken when 30 subjects with HER2+ disease according to blood based NGS assay from either Cohort 2A or the dose optimization stage have been treated at the recommended paclitaxel dose, are evaluable for response, and have been followed for at least 6 weeks. If the ORR per RECIST v1.1 according to investigator assessment is ≥36%, the SMC may recommend that the Phase 3 evaluation of the regimen be initiated in subjects with HER2 amplification in a blood-based NGS assay, given that it is safe and tolerable. Enrollment in Cohort 2B can continue after the initiation of the Phase 3.

Phase 3

Approximately 500 subjects who have HER2 amplification in a blood-based NGS assay will be randomized in approximately an 8:8:1 ratio to either Arm 3A (tucatinib, trastuzumab, ramucirumab, and paclitaxel; 235 subjects), Arm 3B (tucatinib placebo, trastuzumab placebo, ramucirumab, and paclitaxel; 235 subjects), or Arm 3C (tucatinib, trastuzumab placebo, ramucirumab, and paclitaxel; 30 subjects) (Error! Reference source not found.). Efficacy in Arms 3A and 3B will be formally compared to demonstrate the benefit of adding tucatinib plus trastuzumab to standard of care ramucirumab plus paclitaxel. Efficacy in Arm 3C will be analyzed separately. Randomization will be stratified by region (Asia vs Rest of World), time to progression on first-line therapy for locally-advanced unresectable or metastatic disease (<6 months vs ≥6 months), and history of prior gastrectomy (yes vs no). Subjects randomized to Arm 3B will receive a tucatinib placebo and a trastuzumab placebo; subject randomized to Arm 3C will receive a trastuzumab placebo. Subjects, investigators, and staff, and the sponsor study team will be blinded to study arm allocation. Subjects who are screened for the Phase 3 but are found to be HER2-negative according to the blood-based NGS assay, may be enrolled in Cohort 2B of the Phase 2 if it is still enrolling and the subject has HER2-positive disease according to assay of a biopsy obtained after progression on the most recent line of therapy.

In the Phase 3 portion of the study, an Independent Data Monitoring Committee (IDMC) will periodically review relevant aggregate safety data and will make recommendations to the sponsor on the conduct of the study. Safety will also be monitored in an ongoing basis by the sponsor throughout the study.

### Method of Assigning Subjects to Treatment Groups

### Phase 2

Following informed consent and screening assessments, eligible subjects will be assigned to the paclitaxel dose level currently enrolling during the paclitaxel dose optimization stage. Once the paclitaxel recommended dose is established, subjects will be assigned to either Cohort 2A or Cohort 2B based on the results of blood-based NGS assay or, if the former was negative, IHC/ISH assay of a tumor biopsy obtained after progression on the most recent line of systemic therapy.

### Phase 3

Following informed consent and screening assessments, eligible subjects will be randomly assigned to Arms 3A, 3B, and 3C in an approximately 8:8:1 ratio. Randomization will be performed centrally using a system that will assign a unique subject randomization number but will not specify the actual treatment assignment. Randomization procedures are detailed in the Study Manual.
Randomization will be stratified by:
Region of inclusion: Asia versus Rest of World
Time to progression on first-line therapy for locally-advanced unresectable or metastatic disease: <6 months versus ≥6 months
History of prior gastrectomy: yes versus no

### Blinding and Unblinding

Maintaining the blind of the study in the Phase 3 portion is crucial for achieving the study objectives. Unblinding an individual subject's treatment assignment may only occur when one of the following circumstances is applicable:

At the time of study closure, the study treatment assignment will be provided by sponsor to the investigator.

Unblinding a subject's treatment assignment prior to study closure must be limited to emergency circumstances where knowledge of the treatment assignment would affect decisions regarding the clinical management of the subject. In the event of such an emergency circumstance, a formal unblinding procedure, carried out by a third-party organization will be followed to allow the investigator to immediately access a subject's treatment assignment (see Study Manual). Information on study treatment assignment should not be distributed to any other personnel involved in the clinical trial, apart from the study site pharmacist, who will be unblinded to treatment allocation. In the event of any emergency unblinding, the sponsor is to be notified within 24 hours of the occurrence.

Details regarding unblinding procedures are described in the Study Manual.

### Unblinding for Safety Monitoring

Safety data in the Phase 3 portion is monitored by an IDMC. Unblinding of aggregate safety data for ongoing safety monitoring and risk/benefit assessment by the IDMC will be performed through an independent Data Coordinating Center to ensure the integrity of the study.

Suspected unexpected serious adverse reactions will be unblinded in accordance with local regulatory reporting requirements. Pre-specified personnel from the sponsor Drug Safety Department will unblind the identity of study medication for any unexpected (as per the Investigator's Brochure) SAEs that are considered to be related to the blinded study drugs (tucatinib, trastuzumab, or placebo).

### Treatments

### Treatments Administered

Subjects in the Phase 2 portion of the study will receive combination therapy of the investigational medicinal products tucatinib and trastuzumab combined with standard-of-care ramucirumab and paclitaxel. In the Phase 3, subjects will receive either tucatinib and trastuzumab (Arm 3A), tucatinib placebo and trastuzumab placebo (Arm 3B), or tucatinib and trastuzumab placebo (Arm 3C), all combined with ramucirumab and paclitaxel. Study treatment will be given on a 28-day cycle, with tucatinib (or placebo) every day, trastuzumab (or placebo) and ramucirumab on Days 1 and 15, and paclitaxel on Days 1, 8, and 15 (Table 3). In subjects in the Phase 2 undergoing cycle 1 PK assessments, the first tucatinib dose will be given in the evening on Day 1. In this study, subjects are considered to be on study treatment if they are receiving any of the study drugs (tucatinib/placebo, trastuzumab/placebo, ramucirumab, and/or paclitaxel). Cycles are defined by paclitaxel administration, with a new cycle starting whenever the day 1 infusion of paclitaxel is administered. If paclitaxel is discontinued, cycles will be defined as occurring every 28 days from the last day 1 administration of paclitaxel.

In the Phase 2, tucatinib should be dosed at approximately the same time as the start of the paclitaxel infusion on cycle 1 day 8 and cycle 2 day 1, when both tucatinib and paclitaxel PK are assessed. The administration order of the IV study drugs is paclitaxel first, then trastuzumab and ramucirumab, or according to institutional standard of care.

TABLE 3
Treatment schedule

| | | | | Daily | Cycle Day | | |
|---|---|---|---|---|---|---|---|
| Agent | Dose | Route | Cycle | frequency | Day 1 | Day 8 | Day 15 |

Phase 2

| Tucatinib | 300 mg | PO | All | BID | Every day, from Cycle 1 | | |
|---|---|---|---|---|---|---|---|
| | | | | | Day 1^{a} | | |
| Trastuzumab ^{b} | 4 mg/kg | IV | All | Once | X | | X |
| | (6 mg/kg on | | | | | | |
| | Cycle 1 Day 1) | | | | | | |
| Ramucirumab | 8 mg/kg | IV | All | Once | X | | X |
| Paclitaxel | 60 or 80 mg/m² ^{c} | IV | All | Once | X | X | X |

Phase 3

| Arm 3A | | | | | | | |
|---|---|---|---|---|---|---|---|
| Tucatinib | 300 mg | PO | All | BID | Every day, from Cycle 1 | | |
| | | | | | Day 1 | | |
| Trastuzumab ^{b} | 4 mg/kg | IV | All | Once | X | | X |
| | (6 mg/kg on | | | | | | |
| | Cycle 1 Day 1) | | | | | | |
| Ramucirumab | 8 mg/kg | IV | All | Once | X | | X |
| Paclitaxel | Phase 2 | IV | All | Once | X | X | X |
| | recommended | | | | | | |
| | dose | | | | | | |
| Arm 3B | | | | | | | |
| Tucatinib | Not applicable | PO | All | BID | Every day, from Cycle 1 | | |
| placebo | | | | | Day 1 | | |
| Trastuzumab | Not applicable | IV | All | Once | X | | X |
| placebo ^{b} | | | | | | | |
| Ramucirumab | 8 mg/kg | IV | All | Once | X | | X |
| Paclitaxel | 80 mg/m² | IV | All | Once | X | X | X |
| Arm 3C | | | | | | | |
| Tucatinib | 300 mg | PO | All | BID | Every day, from Cycle 1 | | |
| | | | | | Day 1 | | |
| Trastuzumab | Not applicable | IV | All | Once | X | | X |
| placebo ^{b} | | | | | | | |
| Ramucirumab | 8 mg/kg | IV | All | Once | X | | X |
| Paclitaxel | Phase 2 | IV | All | Once | X | X | X |
| | recommended | | | | | | |
| | dose | | | | | | |

^{a}In subjects in the Phase 2 undergoing cycle 1 PK assessments, the first tucatinib dose will be given in the evening on Day 1.
^{b} Trastuzumab may also be given on a weekly basis at 2 mg/kg IV, but only in circumstances where weekly infusions are required to resynchronize with the paclitaxel cycle.
^{c} The paclitaxel dose optimization stage will initially evaluate 60 mg/m² and potentially escalate to 80 mg/m². Alternative dose levels/schedules may be evaluated as recommended by the SMC

### Investigational Product, Dose, and Mode of Administration

### Tucatinib

Tucatinib, an investigational agent under study in this protocol, is a kinase inhibitor that selectively inhibits HER2, and displays limited activity against the related kinase EGFR.

In the Phase 2, tucatinib will be supplied in an open-label manner, by the sponsor. In the Phase 3, treatment allocation to tucatinib or tucatinib placebo will be double-blinded.

Detailed information describing the preparation, administration, and storage of tucatinib is located in the Pharmacy Instructions.

### Description

Tucatinib drug product is supplied as both a coated yellow oval-shaped tablet in a 150 mg dosage strength and a coated yellow round convex tablet in a 50 mg dosage strength. The tablets are manufactured from a drug product intermediate amorphous dispersion of tucatinib in polyvinylpyrrolidone-vinyl acetate copolymer, which is then combined with the pharmaceutical excipients (microcrystalline cellulose, sodium chloride, potassium chloride, sodium bicarbonate, silicon dioxide, crospovidone, and magnesium stearate), and compressed into tablets.

### Dose and Administration

Tucatinib will be administered according to the following:

Route of administration: PO. Dose: Tucatinib 300 mg will be administered PO BID from cycle 1 day 1 onwards.

Dosing schedule: BID on each day of study treatment. Tucatinib or tucatinib placebo should be taken once in the morning and once in the evening, with approximately 8 to 12 hours between doses in the same calendar day. In subjects in the Phase 2 undergoing cycle 1 day 1 PK assessments, tucatinib will not be administered in the morning of cycle 1 day 1; the first dose will be in the evening, after all PK samples have been collected.

In the Phase 3, subjects in Arm 3B receive a tucatinib placebo PO BID.

Dose modifications of tucatinib or placebo are described herein. Subjects will be instructed by the pharmacist or investigator as to the specific number of tablets required for each dose. At each visit during study treatment, subjects will be supplied with the appropriate number of tablets for the number of doses to be taken prior to the next scheduled visit.

It is recommended that if a subject misses a scheduled dose of tucatinib or placebo and less than 6 hours have passed since the scheduled dosing time, the dose should be immediately taken. It is recommended that if more than 6 hours have passed since the scheduled dosing time, the subject should not take the missed dose but should wait and take the next regularly scheduled dose. Tablets may be taken with or without food. Tablets must be swallowed whole and may not be crushed, chewed, or dissolved in liquid. On the day of dosing, the individual unit dose of the tucatinib or placebo tablet may be exposed to ambient temperature for up to 6 hours prior to dose.

Complete dosing instructions will be provided to the pharmacist prior to the initiation of the study. Complete dosing instructions will also be provided to study subjects and will include the minimum times between doses, dosing in relation to meals, and instructions for missed doses.

Subject compliance with investigational study drug dosing instructions will be assessed with the use of subject diaries or pill count and study drug accountability.

### Trastuzumab or Trastuzumab Placebo

### Description

Trastuzumab is a humanized immunoglobulin G1 (IgG1) kappa mAb which binds to the extracellular domain of HER2; it mediates antibody-dependent cellular cytotoxicity by inhibiting proliferation of cells which over express the HER2 protein. Trastuzumab is indicated for adjuvant treatment of HER2-overexpressing node positive or node negative breast cancer, in combination with paclitaxel for first-line treatment of HER2 overexpressing mBC, as a single agent for treatment of HER2-overexpressing breast cancer in patients who have received one or more chemotherapy regimens for metastatic disease, and in combination with cisplatin and capecitabine or 5-fluorouracil, for the treatment of patients with HER2-overexpressing metastatic GEC who have not received prior treatment for metastatic disease.

### Method of Procurement

Trastuzumab is commercially available. In Phase 2, details regarding its sourcing may vary by site and/or region as outlined in other documents such as Clinical Trial Agreements.

In the Phase 3, treatment allocation to trastuzumab or trastuzumab placebo will be double blinded, and will be supplied.

### Dose and Administration

Trastuzumab will be administered on Day 1 and 15 of every 28-day cycle. A loading dose of 6 mg/kg IV will be administered on cycle 1 day 1 followed by 4 mg/kg with each subsequent dose. Trastuzumab placebo will be used in Arms 3B and 3C of the Phase 3. Trastuzumab may also be given on a weekly basis at 2 mg/kg IV once weekly, in order to resynchronize administration to Day 1 and 15 of the 28-day paclitaxel cycle, after discussion with the medical monitor. If dosing of trastuzumab has been held for >4 weeks and the medical monitor has agreed to restart trastuzumab, the IV loading dose of 6 mg/kg should be given per approved dosing instructions. Trastuzumab infusion rates will be per institutional guidelines.

To maintain the blind, subjects assigned to receive trastuzumab placebo will receive an IV infusion that does not contain trastuzumab. Refer to Pharmacy Manual for additional instructions.

### Ramucirumab

### Description

Ramucirumab (CYRAMZA®) is a recombinant human IgG1 mAb with an approximate molecular weight of 147 kDa, produced in genetically engineered mammalian NS0 cells. It is a VEGFR2 antagonist that specifically binds VEGFR2 and blocks binding of VEGFR ligands, VEGF-A, VEGF-C, and VEGF-D. As a result, ramucirumab inhibits ligand stimulated activation of VEGFR2, thereby inhibiting ligand induced proliferation, and migration of human endothelial cells. It is indicated for the treatment of subjects with advanced or metastatic GEC with disease progression on or after prior fluoropyrimidine- or platinum containing chemotherapy as a single agent or in combination with paclitaxel. It is also indicated for subjects with previously-treated metastatic non-small cell lung cancer in combination with docetaxel, in subjects with previously treated mCRC in combination with FOLFIRI, and in subjects with previously treated hepatocellular carcinoma.

### Method of Procurement

Ramucirumab is commercially available and details regarding its sourcing may vary by site and/or region as outlined in other documents such as Clinical Trial Agreements.

### Dose and Administration

Ramucirumab 8 mg/kg will be administered on Days 1 and 15 of each 28 day cycle. Ramucirumab will be administered IV per institutional guidelines, under the direction of the investigator. Ramucirumab is for IV infusion only. Do not administer as an IV push or bolus.

IRRs related to ramucirumab have been observed. To reduce the risk of IRRs with Ramucirumab, subjects will receive premedication/postmedication as described herein.

### Paclitaxel

### Description

Paclitaxel is an antimicrotubule agent that promotes the assembly of microtubules from tubulin dimers and stabilizes microtubules by preventing depolymerization. This stability inhibits the normal dynamic reorganization of the microtubule network, which is essential for vital interphase and mitotic cellular functions. In addition, paclitaxel induces abnormal arrays or bundles of microtubules throughout the cell cycle and multiple asters of microtubules during mitosis.

### Method of Procurement

Paclitaxel is commercially available and details regarding sourcing of paclitaxel may vary by site and/or region as outlined in other documents such as Clinical Trial Agreements.

### Dose and Administration

Paclitaxel will be administered on Days 1, 8, and 15 of each 28-day cycle. Paclitaxel will be administered IV per institutional guidelines, under the direction of the investigator. The initial paclitaxel dose to be evaluated in the Phase 2 dose optimization stage is 60 mg/m²; an 80 mg/m² dose level may be explored. Additional dose levels/schedules may be evaluated as recommended by the SMC. In the Phase 3, paclitaxel will be administered at the recommended dose identified in Phase 2 in Arms 3A and 3C, and at 80 mg/m² in Arm 3B.

Subjects with a history of severe hypersensitivity reactions to products containing polyoxyl 35 castor oil (e.g., cyclosporin for injection concentrate, teniposide for injection concentrate) are not to be treated with paclitaxel.

Injection site and hypersensitivity reactions related to paclitaxel are common. To reduce the risk of these reactions, subjects will receive premedication as described herein. Given the possibility of extravasation, it is advisable to closely monitor the infusion site for possible infiltration during drug administration. Vital signs should be monitored frequently during the paclitaxel infusion.

Initiate concomitant G-CSF as clinically indicated.

Table 4 summarizes the conditions, in terms of AEs and laboratory test abnormalities that must be met for before administering paclitaxel. If the conditions are not met on the planned Day 1 of a cycle, then the paclitaxel infusion should be delayed until the conditions are met. If the conditions are not met on the planned Day 8 or 15, then the paclitaxel infusion should be skipped.

TABLE 4
Criteria for paclitaxel treatment on Day 1, 8, and 15 of each cycle

| AE/abnormality | Day 1 | Day 8 and 15 |
|---|---|---|
| ANC: | ≥1.5 × 10³/μL | ≥1.0 × 10³/μL |

| | |
|---|---|
| Platelets: | ≥75 × 10³/μL |
| Bilirubin: | ≤1.5 × ULN |
| AST/ALT: | ≤3 × ULN ^{a} (<5 × ULN if liver metastases present) |
| Paclitaxel-related | Grade ≤1 or baseline (except for alopecia) |
| AEs: | Anemia Grade ≤2 |

d On cycle 1 day 1, AST/ALT is to be ≤2.5 × ULN (<5 × ULN if liver metastases present).

### Dose Modifications

Dose modification recommendations (including dose holds, dose reduction, or discontinuation of drugs) in response to AEs are described for tucatinib/placebo, for trastuzumab/placebo, for ramucirumab, and for paclitaxel. Dose reductions or treatment interruption/discontinuation for reasons other than those described in the following sections may be made by the investigator if it is deemed in the best interest of subject safety. Whenever possible, these decisions should first be discussed with the study medical monitor.

All AEs and clinically significant laboratory abnormalities should be assessed by the investigator for relationship to tucatinib/placebo, trastuzumab/placebo, ramucirumab, and paclitaxel. An AE may be considered related to any single study drug, any combination of study drugs, or to none of them. In the event that the relationship is unclear, discussion should be held with the study medical monitor, to determine which study drug(s) should be held and/or modified.

The beginning of each cycle is defined by the administration of the Day 1 infusion of paclitaxel. During paclitaxel cycle delays, ramucirumab and trastuzumab administration should continue as planned. When the new paclitaxel cycle starts, ramucirumab should be administered on Day 1, even if it was administered the previous week. Trastuzumab should not be administered on Day 1 if trastuzumab 4 mg/kg was administrated the previous week; instead, to synchronize trastuzumab to a delayed paclitaxel cycle, trastuzumab 2 mg/kg should be given on Day 8 followed by trastuzumab 4 mg/kg on Day 15. If paclitaxel cannot be administered on Day 8 or Day 15 of a cycle, that day is skipped, ramucirumab and trastuzumab are administered as scheduled, and the paclitaxel schedule continues unchanged. If paclitaxel is discontinued, protocol-defined visits will proceed using a 28-day cycle starting from the last paclitaxel Day 1, regardless of dose holds or delays.

Doses held for toxicity will not be replaced. Once reduced, the dose of a study drug should not be re-escalated. Any study drug that requires a delay >4 weeks should be discontinued, unless a longer delay is approved by the study medical monitor. If one or more study drugs are discontinued, study treatment can continue with the remaining study drugs.

### Tucatinib/Placebo Dose Modifications

Up to 3 dose reductions of tucatinib/placebo are allowed (Table 5). Subjects who would require a dose reduction to below 150 mg BID should discontinue treatment with tucatinib/placebo. Dose reductions of larger intervals than those described in Table 5 may be made at the discretion of the investigator with approval by the medical monitor, but dose reductions to below 150 mg BID are not allowed.

TABLE 5
Tucatinib/placebo: Recommended dose
reduction schedule for adverse events

| Dose Reduction Schedule | Tucatinib/Placebo Dose Level |
|---|---|
| Starting dose | 300 mg PO BID ^{a} |
| 1st dose reduction | 250 mg PO BID |
| 2nd dose reduction | 200 mg PO BID |
| 3rd dose reduction | 150 mg PO BID |
| Requirement for further dose reduction | Discontinue treatment |

e Dose reductions of greater intervals than those recommended in this table (i.e., more than 50 mg per dose reduction) may be made if considered clinically appropriate by the investigator and approved by the medical monitor. However, tucatinib/placebo may not be dose reduced below 150 mg BID.

General dose modification guidelines for tucatinib/placebo are provided in Table 6 and Table 7. For subjects with documented Gilbert's disease, contact the medical monitor for guidance regarding dose modifications for LFT abnormalities.

TABLE 6
Dose modifications for clinical adverse events related to tucatinib/placebo

| | |
|---|---|
| Adverse Reactions | Tucatinib Dose Modification |

Diarrhea

| | |
|---|---|
| Grade 3 without anti-diarrheal treatment | Initiate or intensify appropriate medical therapy. |
| | Hold tucatinib until recovery to ≤Grade 1 or |
| | baseline |
| Grade 3 with anti-diarrheal treatment | Initiate or intensify appropriate medical therapy. |
| | Hold tucatinib until recovery to ≤Grade 1 or |
| | baseline |
| | Reduce tucatinib dose. |
| Grade 4 (life-threatening consequences; | Permanently discontinue tucatinib treatment. |
| urgent intervention indicated) | |

Other adverse reactions

| | |
|---|---|
| Grade 3 | Hold tucatinib until recovery to ≤Grade 1 or |
| | baseline |
| | Reduce tucatinib dose |
| Grade 4 | Permanently discontinue tucatinib. |

TABLE 7
Dose modifications of tucatinib/placebo for LFT abnormalities,
regardless of relationship to tucatinib/placebo

| Laboratory Abnormality | Tucatinib Dose Modification |
|---|---|
| Bilirubin elevation >1.5-3 × ULN | Hold tucatinib until recovery to ≤1.5 × ULN |
| Bilirubin elevation >3-10 × ULN | Hold tucatinib until recovery to ≤1.5 × ULN |
| | Reduce tucatinib dose. |
| Bilirubin elevation >10 × ULN | Permanently discontinue tucatinib. |
| ALT or AST elevation >5-20 × ULN | Hold tucatinib until recovery to ≤3 × ULN or |
| | return to baseline level in subjects with known |
| | liver metastasis |
| | Reduce tucatinib dose. |
| ALT or AST elevation >20 × ULN | Permanently discontinue tucatinib. |
| ALT or AST >3 × ULN AND | Permanently discontinue tucatinib. |
| bilirubin >2 × ULN | |

### Trastuzumab/Placebo Dose Modifications

In the event of Grade ≥3 trastuzumab-related AEs, hold trastuzumab until the AE has resolved to Grade ≤1 or pretreatment levels and initiate or intensify applicable medical therapy, as appropriate. Resume trastuzumab at the same dose; the trastuzumab/placebo dose may not be reduced. If dosing of trastuzumab is held for >4 weeks and the medical monitor has agreed to restart trastuzumab, the IV loading dose of 6 mg/kg should be given per approved dosing instructions.

Trastuzumab dose modification guidelines for left ventricular dysfunction and cardiomyopathy, IRR, and for hypersensitivity reactions herein.

### Left Ventricular Dysfunction and Cardiomyopathy

Trastuzumab can cause left ventricular cardiac dysfunction, arrhythmias, hypertension, disabling cardiac failure, cardiomyopathy, and cardiac death. Trastuzumab can also cause asymptomatic decline in LVEF.

Trastuzumab/placebo dose modification guidelines for left ventricular dysfunction, regardless of relationship to study drug, are provided in Table 8.

TABLE 8
Trastuzumab and trastuzumab placebo dose modification guidelines for left ventricular dysfunction

| | | LVEF 40% to ≤45% | LVEF 40% to ≤45% | |
|---|---|---|---|---|
| | | and decrease ≥10% | and decrease <10% | |
| Symptomatic CHF | LVEF <40% | points from baseline | points from baseline | LVEF >45% |
| Discontinue | Do not administer | Do not administer | Continue | Continue |
| trastuzumab/placebo | trastuzumab/placebo | trastuzumab/placebo | treatment with | treatment with |
| | | | trastuzumab/ | trastuzumab/ |
| | | | placebo | placebo |
| | Repeat LVEF | Repeat LVEF | Repeat LVEF | |
| | assessment within | assessment within | assessment | |
| | 3 weeks. | 3 weeks. | within 3 weeks | |
| | If LVEF <40% is | If the LVEF has not | | |
| | confirmed, | recovered to within | | |
| | discontinue | 10% points from | | |
| | trastuzumab/placebo | baseline, discontinue | | |
| | | trastuzumab/placebo | | |

### Infusion-Related Reactions

Symptoms of IRR occurring after trastuzumab administration include fever and chills, and on occasion included nausea, vomiting, pain (in some cases at tumor sites), headache, dizziness, dyspnea, hypotension, rash, and asthenia. In severe cases, symptoms have included bronchospasm, anaphylaxis, angioedema, hypoxia, and severe hypotension, usually reported during or immediately following the initial infusion. However, the onset and clinical course are variable, including progressive worsening, initial improvement followed by clinical deterioration, or delayed post-infusion events with rapid clinical deterioration. For fatal events, death occurred within hours to days following a serious IRR.

Interrupt trastuzumab infusion in all subjects experiencing dyspnea or clinically significant hypotension, and administer supportive therapy (which may include epinephrine, corticosteroids, diphenhydramine, bronchodilators, and oxygen). Subjects should be evaluated and carefully monitored until complete resolution of signs and symptoms. In subsequent infusions, premedicate subjects with antihistamines and/or corticosteroids.

Discontinue trastuzumab in subjects with Grade 3 to 4 IRR.

### Hypersensitivity Reactions

Allergic/hypersensitivity reactions are characterized by adverse local or general responses from exposure to an allergen (NCI CTCAE version 5.0). For purposes of this study, allergic/hypersensitivity reactions are differentiated from IRRs by being defined as occurring >24 hours after infusion of trastuzumab. Allergic/hypersensitivity reactions may manifest in the same manner as IRRs, i.e., a combination of signs or symptoms including fever, rigors, flushing, itching, various types of rash, urticaria, dyspnea, nausea, vomiting, back or abdominal pain, and/or hypotension.

Anaphylaxis is a severe, life-threatening, generalized or systemic allergic/hypersensitivity reaction. Anaphylaxis is characterized by an acute inflammatory reaction resulting from the release of histamine and histamine-like substances from mast cells, causing a hypersensitivity immune response. Clinically, it presents with breathing difficulty, dizziness, hypotension, cyanosis, and loss of consciousness and may lead to death (Rosello 2017).

If anaphylaxis occurs, administration of trastuzumab should be immediately and permanently discontinued.

### Ramucirumab Dose Modifications

Up to 2 dose reductions of ramucirumab will be allowed (Table 9). Subjects who would require a dose reduction to below 5 mg/kg should discontinue treatment with ramucirumab.

TABLE 9
Ramucirumab: Recommended dose reduction
schedule for adverse events

| Dose Reduction Schedule | Ramucirumab Dose Level |
|---|---|
| Starting dose | 8 mg/kg |
| 1st dose reduction | 6 mg/kg |
| 2nd dose reduction | 5 mg/kg |
| Requirement for further dose reduction | Discontinue treatment |

General dose modification guidelines for ramucirumab are provided in Table 10. Ramucirumab should be held for 28 days prior to any surgery, and resumed no sooner than 28 days after surgery, once the wound is fully healed and following discussion with the medical monitor.

Ramucirumab dose modification guidelines for hypertension, IRR, proteinuria, and nephrotic syndrome, impaired wound healing, and for reversible posterior leukoencephalopathy syndrome (RPLS) herein.

TABLE 10
Dose modifications for clinical adverse events

| | |
|---|---|
| Clinical Adverse Event | Ramucirumab Dose Modification |

Regardless of relationship to ramucirumab

| | |
|---|---|
| Grade 3-4 hemorrhage | Discontinue ramucirumab |
| Gastrointestinal perforation any grade | Discontinue ramucirumab |
| Wound healing complications of any grade that | Discontinue ramucirumab |
| require medical intervention | |
| Arterial Thromboembolic Events any grade | Discontinue ramucirumab |
| Grade 3-4 hypertension | Hold until controlled by medical |
| | management |
| Grade 3-4 hypertension not controlled by | Discontinue ramucirumab |
| antihypertensive therapy | |
| Hypertensive crisis or hypertensive encephalopathy | |
| Proteinuria | |
| 1st occurrence (≥2 g per 24 hours) | Hold until <2 g per 24 hours. Reduce |
| | dose |
| Reoccurrence after dose reduction (≥2 g per 24 | Hold until <2 g per 24 hours. Reduce |
| hours) | dose |
| ≥3 g per 24 hours or in the setting of nephrotic | Discontinue ramucirumab |
| syndrome | |
| RPLS confirmed by MRI | Discontinue ramucirumab |
| Spontaneous development of fistula | Discontinue ramucirumab |
| Hepatic encephalopathy or hepatorenal syndrome | Discontinue ramucirumab |

Related to ramucirumab

| | |
|---|---|
| IRR | |
| Grade 1-2 | Reduce infusion rate by 50% |
| Grade 3-4 | Discontinue ramucirumab |

### Hypertension

Control hypertension prior to initiating treatment with ramucirumab. Monitor blood pressure every two weeks or more frequently as indicated during treatment. Withhold ramucirumab for severe hypertension until medically controlled.

Permanently discontinue ramucirumab for medically significant hypertension that cannot be controlled with antihypertensive therapy or in subjects with hypertensive crisis or hypertensive encephalopathy.

### Infusion-Related Reactions

Symptoms of IRR occurring after ramucirumab administration have included rigors/tremors, back pain/spasms, chest pain and/or tightness, chills, flushing, dyspnea, wheezing, hypoxia, and paresthesia. In severe cases, symptoms included bronchospasm, supraventricular tachycardia, and hypotension.

Premedicate prior to each ramucirumab infusion. Monitor subjects during the infusion for signs and symptoms of IRR in a setting with available resuscitation equipment.

Reduce the infusion rate by 50% for Grade 1 to 2 IRR. Permanently discontinue ramucirumab for Grade 3 to 4 IRRs.

### Proteinuria and Nephrotic Syndrome

Monitor proteinuria by urine dipstick and/or urinary protein creatinine ratio. If the result of the urine dipstick is 2+ or greater, perform a 24-hour urine collection for protein measurement. Withhold ramucirumab for urine protein levels that are 2 or more grams over 24 hours. Reinitiate ramucirumab at a reduced dose once the urine protein level returns to less than 2 grams over 24 hours. Permanently discontinue ramucirumab for urine protein levels greater than 3 grams over 24 hours or in the setting of nephrotic syndrome.

### Impaired Wound Healing

Withhold ramucirumab for 28 days prior to surgery. Do not administer ramucirumab for at least 28 days following a major surgical procedure, until the wound is fully healed, following discussion with the medical monitor. Discontinue ramucirumab in subjects who develop wound healing complications that require medical intervention.

### Reversible Posterior Leukoencephalopathy Syndrome

In the event of RPLS, confirm the diagnosis of RPLS with magnetic resonance imaging (MRI) and permanently discontinue ramucirumab.

### Paclitaxel Dose Modifications

The paclitaxel dose can be reduced by increments of 10 mg/m² (i.e., reductions to 70 mg/m² then to 60 mg/m² for a subject initially receiving 80 mg/m²); however, subjects who would require a dose reduction to below 60 mg/m² should discontinue treatment with paclitaxel. Dose reductions are implemented only at the start of a cycle, not on Day 8 or 15.

General dose modification guidelines for paclitaxel are provided in Table 11 and Table 12.

Paclitaxel dose modification guidelines for hepatotoxicity and for hypersensitivity herein.

TABLE 11
Paclitaxel dose modifications for clinical adverse events related to paclitaxel

| Adverse Reaction | Paclitaxel Dose Modification |
|---|---|
| Paclitaxel-related | Delay Day 1 administration until recovery to Grade ≤1 |
| non-hematological AEs | or baseline or skip Day 8 or 15 |
| Grade ≥3 | Reduce paclitaxel dose in next cycle |
| Grade 3-4 peripheral neuropathy | Delay Day 1 administration until recovery to Grade ≤1 |
| | or baseline or skip Day 8 or 15 |
| | Reduce paclitaxel dose in next cycle |
| Grade 3-4 hypersensitivity reaction | Discontinue paclitaxel |

TABLE 12
Paclitaxel dose modifications for hematological abnormalities,
regardless of relationship to paclitaxel

| Hematological Abnormality | Paclitaxel Dose Modification |
|---|---|
| ANC <1.5 × 10³/μL | Delay Day 1 administration until recovery to ≥1.5 × |
| | 10³/μL |
| ANC <1.0 × 10³/μL | Delay Day 1 administration recovery to ≥1.5 × 10³/μL or |
| | skip Day 8 or 15 administration |
| Platelet count <75 × 10³/μL | Delay Day 1 administration recovery to ≥75 × 10³/μL or |
| | skip Day 8 or 15 administration |
| Grade 4 hematological | Reduce paclitaxel dose in next cycle |
| abnormalities | |

### Hepatotoxicity

Dose modification for paclitaxel in the case of LFT abnormalities, regardless of relationship to study drug, are summarized in Table 13. A rise in indirect bilirubin with a normal direct bilirubin believed to be attributable to Gilbert's disease does not require change in dose or a drug hold.

TABLE 13
Paclitaxel dose modification guidelines for liver function
abnormalities, regardless of relationship to paclitaxel

| LFT abnormality | Action |
|---|---|
| Bilirubin elevation >1.5-3 × ULN | Delay Day 1 administration until recovery to ≤1.5 × |
| | ULN or skip Day 8 or 15 infusions |
| Bilirubin elevation >3-10 × ULN | Delay Day 1 administration until recovery to ≤1.5 × |
| | ULN or skip Day 8 or 15 infusions |
| | Reduce paclitaxel dose in next cycle |
| Bilirubin elevation >10 × ULN | Permanently discontinue paclitaxel. |
| AST or ALT elevation >5-20 × ULN | Delay Day 1 administration until recovery to ≤3 × |
| | ULN or return to baseline level in subjects |
| | with known liver metastasis or skip Day 8 or 15 |
| | infusions |
| | Reduce paclitaxel dose |
| AST or ALT elevation >20 × ULN | Permanently discontinue paclitaxel |
| ALT or AST >3 × ULN AND | Permanently discontinue paclitaxel |
| bilirubin >2 × ULN | |

### Hypersensitivity Reactions

Paclitaxel treatment interruption is not required for minor symptoms of hypersensitivity, such as flushing, skin reactions, dyspnea, hypotension, or tachycardia.

Paclitaxel should be discontinued and aggressive symptomatic therapy applied in the event of severe reactions, such as hypotension requiring treatment, dyspnea requiring bronchodilators, angioedema, or generalized urticaria.

### Required Premedication and Postmedication

Suggested premedication to be administered prior to each paclitaxel infusion is presented in Table 14. Paclitaxel premedication can be adjusted according to standard institutional practice.

TABLE 14
Paclitaxel premedication

| | | |
|---|---|---|
| | | Administration time |
| Pre-medication | Dose | prior to paclitaxel |

| | | |
|---|---|---|
| Dexamethasone | 8-10 mg PO or | 30 to 60 min |
| | 8-10 mg IV | |

| | | | |
|---|---|---|---|
| Diphenhydramine or | 12.5-50 | mg IV | 30 to 60 min |
| equivalent | | | |
| Ranitidine | 50 | mg IV | 30 to 60 min |

Ramucirumab pre-medication with an IV histamine-1 receptor antagonist (e.g., diphenhydramine hydrochloride) may be given prior to each ramucirumab infusion at the discretion of the investigator. For subjects who have experienced a Grade 1 or 2 IRR considered at least possibly related to ramucirumab, premedicate with a histamine-1 receptor antagonist, dexamethasone (or equivalent), and acetaminophen prior to each ramucirumab infusion.

Subjects who have experienced dyspnea or clinically significant hypotension related to trastuzumab during or following the previous infusion should be premedicated with antihistamines and/or corticosteroids prior to subsequent trastuzumab infusions.

### Duration of Treatment

Study treatment will continue until unacceptable toxicity, disease progression, withdrawal of consent, death, or study closure. If a study drug (tucatinib/placebo, trastuzumab/placebo, ramucirumab, or paclitaxel) is discontinued, study treatment can continue with remaining study drug(s).

In the absence of clear evidence of radiographic progression per RECIST version 1.1, all efforts should be made to continue treatment until unequivocal evidence of radiologic progression per RECIST version 1.1 occurs. No crossover will be allowed.

### Study Assessments

Screening/Baseline Assessments

Screening/Baseline assessments will be conducted to establish study baseline status and determine study eligibility.

Subject medical history includes a thorough review of significant past medical history, current conditions, any treatment for prior malignancies and response to prior treatment, and any concomitant medications.

The following assessments are required for all subjects at screening and/or baseline: physical exam, height, vital signs, weight, ECOG performance status, CT with contrast/PET-CT/MRI scan for baseline disease assessment, CBC with differential, serum chemistry panel, coagulation panel, urinalysis, ECG, echocardiogram/MUGA, Hepatitis B and C screening, blood sample for biomarker assay, and serum or urine p-hCG pregnancy test (for females of childbearing potential).

### Evaluation of HER2 Status at Screening

A blood sample will be drawn to establish baseline HER2 amplification using an NGS assay, performed at a central laboratory prior to randomization.

Archival tumor blocks (or freshly-cut slides, following consultation with the medical monitor) sampled following progression during/after the most recent line of therapy, or other archival biopsies performed prior to the first line therapy for advanced disease, are to be collected at screening. Tissue samples obtained via resection, excision, punch (skin lesions only), or core needle from a tumor site are suitable for testing. Fine needle aspiration, brushing, cell pellets from pleural effusion, forceps, and lavage samples are not acceptable. Tumor tissue should be of good quality based on total and viable tumor content; e.g., samples should contain a minimum of 100 tumor cells that preserve cellular context and tissue architecture, regardless of the needle gauge used to collect the sample or the retrieval method. See the Laboratory Manual for details concerning tissue samples.

HER2 expression in the biopsy will be evaluated using tissue-based NGS and according to the 2016 guideline of the American Society of Clinical Oncology (ASCO)/College of American Pathologists (CAP) “HER2 Testing and Clinical Decision Making in Gastroesophageal Adenocarcinoma”, and the package insert of FDA-approved tests for IHC and ISH.

### Response/Efficacy Assessments

Disease response to study treatment and the occurrence of disease progression will be determined according to RECIST version 1.1, as assessed by the investigator and by the BICR (Phase 3). Radiographic scans and additional imaging assessments (if applicable) will be performed at protocol-specified time points. Clinical management decisions will be based on local investigator assessment to ensure that treatment decisions are made in a timely manner; results of centralized review will not be available to investigators for clinical decision making.

Disease assessments will be performed at screening, and every 6 weeks for the first 36 weeks then every 9 weeks, irrespective of dose holds or interruptions. Subjects that discontinue study treatment for reasons other than documented progressive disease will continue to have disease assessments at least every 9 weeks until the occurrence of documented progression per RECIST version 1.1, death, withdrawal of consent, or study closure.

All known sites of metastatic or locally-advanced unresectable disease should be assessed by radiographic imaging at Screening/Baseline to document sites of disease and tumor burden. Imaging, preferably by high quality spiral contrast CT scan (with oral and/or IV contrast), should include the chest, abdomen, and pelvis, at a minimum; PET/CT (if high quality CT scan is included) and/or MRI scan may also be done as appropriate. If a CT scan with contrast is contraindicated (i.e., in subjects with contrast allergy or impaired renal clearance), a non-contrast CT scan of the chest may be performed instead, with MRI scans of the abdomen and pelvis. At the investigator's discretion, other appropriate imaging (e.g., nuclear bone scan imaging for bone lesions) should be used to assess additional known sites of measurable disease. The same imaging modalities employed in Screening/Baseline should be used for all subsequent response assessments during study treatment and in the follow-up period, unless otherwise clinically indicated. If any other radiographic or assessment exam, including pathology from any on-study biopsies or procedures, is conducted per standard of care, the assessment information will be collected in the CRF. In the Phase 3, all imaging will be collected for retrospective BICR.

In the event of equivocal progression, for example a new lesion which is small in size (defined as an equivocal new lesion) and no imminent threat to subject safety, all efforts should be made to continue the subject until unequivocal radiologic progression or clinical progression is documented. Demonstration of an unequivocal new lesion constitutes disease progression.

In the Phase 2, subjects will be considered evaluable for response if they meet the following 3 criteria: (1) had baseline disease assessment, (2) received study treatment, and (3) had post-baseline disease assessment or discontinued treatment due to documented disease progression, clinical progression, treatment-related AE(s), or death.

In the Phase 3, all randomized subjects with measurable disease at baseline will be considered evaluable for response.

Subjects' clinical data must be available for CRF source verification. Copies of tumor images must be made available for review by the sponsor (or its designee) upon request. In the Phase 3, all imaging will be submitted or uploaded for retrospective BICR as soon as reasonably possible (e.g., within approximately 2 weeks) following the date of assessment. Refer to the Study Manual for instructions on collecting and submitting tumor imaging studies to the third-party imaging core laboratory for BICR.

Pharmacokinetic Assessments

Blood samples for tucatinib, ONT-993, paclitaxel, and paclitaxel metabolites PK assessment will be collected at protocol-defined timepoints; Error! Reference source not found. specifies PK and biomarker collection timepoints for all subjects in Phase 2 and Phase 3, while Error! Reference source not found. specifies additional PK collection timepoints for subjects in the paclitaxel dose optimization stage and the first 6 subjects with and without gastrectomies (without maintenance of the pylorus).

In all subjects in the paclitaxel dose optimization stage and in the first 6 subjects in the Phase 2 dose expansion stage with a gastrectomy, tucatinib and ONT-993 concentrations will be sampled on cycle 1 day 8 and cycle 2 day 1; concentrations of paclitaxel and its metabolites will be sampled on cycle 1 days 1 and 8 and cycle 2 day 1. Subjects with gastrectomies can come from Cohort 2A or Cohort 2B.

In all subjects in the Phase 2 and Phase 3, tucatinib trough drug concentrations will be sampled on Day 1 of Cycles 2 to 6 prior to administration of tucatinib or placebo.

Plasma concentrations of tucatinib, ONT 993, paclitaxel, and its metabolites will be determined using validated liquid chromatography (LC)-mass spectrometry (MS)/MS methods. PK parameters of tucatinib, paclitaxel, and their respective metabolites will be calculated using standard noncompartmental methods. PK parameters to be estimated may include, but are not limited to: AUCₗₐₛₜ, Cₘₐₓ, C_{trough}, Tₘₐₓ, and MR_{AUC}.

Trough PK samples should continue to be collected on schedule regardless of dose holds or interruptions. The cycle 1 and cycle 2 post-dose samples should not be collected during dose hold or interruptions.

### PRO and HCRU Assessments

The EORTC QLQ-C30, EORTC QLQ-OG25, and EQ-5D-5L patient-reported outcome measures will be administered to assess GEC symptoms and HRQoL/health status information. PROs will be assessed, in the Phase 3 portion, predose on cycle 1 day 1, predose on Day 1 of every second cycle (Cycles 2, 4, 6, etc.) until discontinuation of all study treatment, at the end-of-treatment (EOT) visit, and at each follow-up visit until the occurrence of documented progression, death, withdrawal of consent, or study closure. HCRU data will also be collected during treatment and follow-up, including procedures that occur on study, length of stay, hospitalizations, ED visits, planned/unplanned provider visits, medication use, radiology, and other treatments or procedures.

### Biomarker Assessments

HER2 status will be determined by blood-based NGS assay and by IHC/ISH assay of tumor biopsies (IHC3+ or IHC2+/ISH+). Additional biomarker assessments may include HER2 status by tissue-based NGS as well as an exploratory assessment of HER2 mutations or other mutations as potential biomarkers of response. Additional exploratory analyses including but not limited to IHC and NGS analysis may be performed to interrogate biomarkers that are associated with tumor growth, survival, and resistance to targeted therapeutics. This assessment may enable the correlation of additional biomarkers with treatment outcome and may ultimately guide or refine patient selection strategies to better match tucatinib regimens with tumor phenotype/genotype in the future.

### Safety Assessments

Safety assessments will include the surveillance and recording of AEs and SAEs, physical examination findings, vital signs including weight, electrocardiograms (ECGs), concomitant medications, pregnancy testing, and laboratory tests. Assessment of cardiac ejection fraction will be performed using MUGA scan or echocardiogram.

### Statistical Methods

### Sample Size Considerations

### Phase 2:

The preliminary activity of the study regimen will be formally evaluated in approximately 30 response-evaluable subjects from the dose optimization stage or the dose expansion stage (Cohort 2A) who are HER2+ by the blood-based NGS assay and who were treated at the paclitaxel recommended dose.

The Phase 3 may be initiated if the observed ORR per investigator is ≥36%. With the sample size of 30, it is expected that at least 11 responders will be observed if the underlying ORR is ≥36%. The point estimate and 95% CI for ORR under different underlying ORR for the sample size of 30 is as follows:

| | Number of responses | Lower bound | Upper bound |
|---|---|---|---|
| ORR | in 30 subjects | of 95% CI | of 95% CI |
| 30% | 9 | 14.7% | 49.4% |
| 36% | 11 | 19.4% | 55.5% |
| 40% | 12 | 22.7% | 59.4% |
| 47% | 14 | 28.6% | 66.0% |
| 50% | 15 | 31.3% | 68.7% |

Response-evaluable subjects in the Phase 2 include all subjects who meet all following criteria: (1) had baseline disease assessment, and (2) received study treatment, and (3) had post baseline assessment or discontinued treatment due to documented disease progression, clinical progression, toxicity, or death.

### Phase 3:

The sample size for this portion of study was calculated based on maintaining 90% power for the dual primary endpoint of PFS with an alpha of 0.02 and 88% power for the dual primary endpoint of OS with an alpha of 0.03. For PFS, 317 events from Arm 3A or 3B are required with 90% power to detect a hazard ratio of 0.67 (4.5 months median PFS in Arm 3B versus 6.75 months in Arm 3A) using a 2-sided log-rank test and alpha of 0.02. For OS, 354 events from Arm 3A or 3B are required with 88% power to detect a hazard ratio of 0.70 (10 months median OS in Arm 3B versus 14.3 months in Arm 3A) using a 2-sided log-rank test and alpha of 0.03. The 2 primary endpoints will be evaluated using parallel testing, with alpha recycling if only one of them meets statistical significance.

Approximately 500 subjects will be randomized in approximately an 8:8:1 ratio to Arm 3A, Arm 3B, or Arm 3C. Approximately 470 subjects are expected to be randomized to the formal comparison of Arm 3A and Arm 3B. Assuming an accrual period of 30 months and a 5% yearly drop-out rate, it is expected that 317 PFS events and 354 OS events out of the 470 subjects will be observed approximately 25 and 39 months after first subject randomized, respectively.

### Interim Analyses

The SMC will undertake safety and PK analyses once the first 6 subjects evaluable for DLT in each dose level in the paclitaxel dose optimization stage have been followed for at least 1 cycle. If alternative paclitaxel dose levels/schedule are evaluated, the SMC will undertake similar assessments.

In the Phase 3, there is no formal interim analysis planned for PFS. An interim efficacy analysis for OS is planned at the time of final analysis for PFS. Approximately 61% of the total OS events are expected to have occurred by the time of the interim analysis. The stopping boundary will be determined using Lan-DeMets spending functions with O'Brien and Fleming boundaries.

### Analysis Methods

For Phase 2, “treatment group” will designate each dose level evaluated in the Phase 2 for analyses done in the All-Treated analysis set. For analyses using the Recommended Dose analysis set, 2 treatment groups will be presented: 1) subjects with HER2+ disease according to blood-based NGS assay treated at the paclitaxel recommended dose in the dose optimization stage or Cohort 2A, and 2) subjects who have HER2-negative disease according to blood-based NGS but HER2+ disease according to IHC/ISH assay of tumor biopsies treated at the paclitaxel recommended dose in the dose optimization stage or Cohort 2B.

For Phase 3, Arms 3A and 3B will be compared in the ITT analysis set and the Safety Analysis set; Arm 3C will be separately evaluated in these analysis sets.

In the Phase 2, efficacy will be summarized by treatment group in the Recommended Dose analysis set; safety will be summarized by treatment group in the All Treated analysis set. In the Phase 3, efficacy and PROs will be summarized by treatment group in the ITT analysis set; safety will be summarized by treatment group in the Safety Analysis set.

In the Phase 2, ORR, confirmed ORR, DOR, DCR and PFS per investigator will be summarized by treatment group. In the Phase 3, for the primary endpoints of PFS per investigator and OS, Arm 3A and Arm 3B will be compared using a 2-sided stratified log-rank test. Estimation of the hazard ratio will be based upon the stratified Cox regression model. PFS per investigator and OS will also be summarized using the Kaplan-Meier method, which will be used to estimate the time to event curves, including the median and milestone estimates. All subjects randomized to Arms 3A and 3B in the Phase 3 portion of the study will be included in the primary analysis of PFS and OS.

If only one of the two primary endpoints are statistically significant, the unused alpha can be passed to the other one. If both PFS per investigator and OS are statistically significant, then confirmed ORR per investigator among subjects with measurable disease in the Phase 3 portion will be formally compared between two treatment arms at the two-sided alpha level of 0.05, using a stratified Cochran-Mantel-Haenszel test.

The other secondary efficacy endpoints for the Phase 3 portion, including PFS, ORR per investigator, confirmed ORR per BICR, and DOR and DCR per BICR and per investigator, will be summarized by treatment arm. The PFS per investigator, OS, and confirmed ORR per investigator will be summarized separately for Arm 3C.

In the Phase 3, assessments based on the EORTC-QLQ-C30, EORTC QLQ OG25, EQ 5D 5L and HCRU data will summarized using descriptive statistics by treatment group for Arms 3A and 3B of the ITT set. PRO scores will be analyzed using longitudinal models. All subscales and individual item scores will be tabulated. Descriptive summaries of observed data at each scheduled assessment timepoint may be presented. Time to deterioration will be assessed in specific pre specified single items from either the EORTC QLQ-C30 or EORTC QLQ OG25; deterioration is defined as a 10-point increase from baseline in the symptom scales and a 10-point decrease from baseline for overall HRQoL.

Safety will be assessed through summaries of AEs, changes in laboratory test results, and changes in cardiac ejection fraction results. AEs will be classified by system organ class (SOC) and preferred term using the Medical Dictionary for Regulatory Activities (MedDRA); AE severities will be classified using the CTCAE version 5 criteria. All collected AE data will be listed.

Worst post-baseline laboratory values (hematology, coagulation, chemistry, and liver function) and change from baseline will be summarized. Abnormal laboratory values (relative to respective normal ranges) will be flagged in listings. The frequency and percentage of subjects with post-baseline clinically significant vital signs will be summarized. Cardiac ejection fraction data will be summarized for the all-treated subjects in Phase 2, by initial dose level, and for the Phase 3 by treatment group.

Extent of exposure for study drugs including frequency of dose holding, dose reductions, and dose discontinuations, as well as treatment compliance (percent of actual to planned dosing) will be summarized.

Statistical analysis methods for PK will include descriptive statistics on plasma concentrations and PK parameters, as well as exploratory analysis of the geometric mean ratios and 90% CIs of AUCₗₐₛₜ and Cₘₐₓ for paclitaxel and its metabolites between cycle 1 day 1, cycle 1 day 8, and cycle 2 day 1, and exploratory analysis of the geometric mean ratios and 90% CIs of tucatinib and ONT-993 between cycle 1 day 8 and cycle 2 day 1, and between subjects with and without gastrectomy.

### Study Population

This study will enroll subjects with locally-advanced unresectable or metastatic HER2+ GEC who have received prior treatment with a HER2-directed antibody. Subjects will meet all of the enrollment criteria specified herein to be eligible for this study. Eligibility criteria may not be waived by the investigator and are subject to review in the event of a good clinical practice audit and/or health regulatory authority inspection.

### Inclusion Criteria

1. Histologically or cytologically confirmed diagnosis of locally-advanced unresectable or metastatic HER2+ gastric or gastroesophageal junction adenocarcinoma
2. HER2+ disease at screening, as follows:
   a. Phase 2 paclitaxel dose optimization stage: HER2 amplification in a blood-based NGS assay performed at a central laboratory or centrally-confirmed HER2 overexpression/amplification in a tumor biopsy obtained after progression on the most recent line of systemic therapy, evaluated following the package insert of FDA-approved tests for IHC and ISH (IHC3+ or IHC2+/ISH+)
   b. Phase 2 dose expansion stage:
      i. Cohort 2A: HER2 amplification in a blood-based NGS assay performed at a central laboratory
      ii. Cohort 2B: centrally-confirmed HER2 overexpression/amplification in a tumor biopsy obtained after progression on the most recent line of systemic therapy, evaluated following the package insert of FDA-approved tests for IHC and ISH (IHC3+ or IHC2+/ISH+)
   c. Phase 3: HER2 amplification in a blood-based NGS assay performed at a central laboratory
3. Can supply archival tumor tissue for central assay; if an archival sample is not available, the subject may be eligible, following approval by the medical monitor.
4. History of prior treatment with a HER2-directed antibody
5. Progressive disease during or after first-line therapy for locally-advanced unresectable or metastatic GEC
6. Phase 2: Measurable disease according to RECIST version 1.1 Phase 3: Measurable or non-measurable disease according to RECIST version 1.1
7. Age ≥18 years, or considered an adult by local regulations, at time of consent
8. Eastern Cooperative Oncology Group (ECOG) performance status score of 0 or 1
9. Life expectancy of at least 3 months, in the opinion of the investigator
10. Adequate hepatic function as defined by the following:
   a. Total bilirubin ≤1.5×ULN, except for subjects with known Gilbert's disease, who may enroll if the conjugated bilirubin is ≤1.5×ULN
   b. Transaminases (AST and ALT)≤2.5×ULN (≤5×ULN if liver metastases are present)
11. Adequate baseline hematologic parameters as defined by:
   a. ANC≥1.5×10³/μL
   b. Platelet count ≥100×10³/μL; subjects with a stable platelet count from 75-100×10³/μL may be included with approval from the medical monitor
   c. Hemoglobin ≥9 g/dL; subjects with hemoglobin ≥8-9 g/dL may be included with approval from the Medical Monitor
   d. In subjects transfused before study entry, transfusion must be ≥14 days prior to start of therapy to establish adequate hematologic parameters independent from transfusion support
12. Estimated glomerular filtration rate (GFR)≥50 mL/min/1.73 m² using the Modification of Diet in Renal Disease (MDRD) study equation as applicable.
13. International normalized ratio (INR)≤1.5, and prothrombin time (PT) and partial thromboplastin time (PTT)/activated partial thromboplastin time (aPTT)≤1.5×ULN.
14. Left ventricular ejection fraction (LVEF)≥50% as assessed by echocardiogram or multi-gated acquisition scan (MUGA) documented within 4 weeks prior to first dose of study treatment.
15. Urinary protein of ≤1+ on dipstick or routine urinalysis. If dipstick or routine analysis indicates proteinuria ≥2+, then a 24-hour urine must be collected and must demonstrate <1000 mg of protein in 24 hours to allow participation in study
16. The subject must provide written informed consent
17. Subject must be willing and able to comply with study procedures, laboratory tests, and other requirements of the study

### Exclusion Criteria

1. Subjects with squamous cell or undifferentiated GEC
2. Having received more than 1 line of prior systemic therapy for locally-advanced unresectable or metastatic disease
3. Having received taxanes ≤12 months prior to enrollment, prior treatment with ramucirumab, or prior treatment with tucatinib, lapatinib, neratinib, afatinib, or any other investigational anti-HER2 and/or anti-EGFR tyrosine kinase inhibitor, or with T-DM1, DS8201a, or any other HER2-directed antibody-drug conjugate
4. History of exposure to the following cumulative doses of anthracyclines:
   e. Doxorubicin >360 mg/m²
   f. Epirubicin >720 mg/m²
   g. Mitoxantrone >120 mg/m²
   h. Idarubicin >90 mg/m²
   i. Liposomal doxorubicin (e.g. Doxil, Caelyx, Myocet) >550 mg/m²
5. History of allergic reactions to trastuzumab, ramucirumab, paclitaxel, or compounds chemically or biologically similar to tucatinib, except for Grade 1 or 2 IRR to trastuzumab or ramucirumab that were successfully managed, or known allergy to any of the excipients in the study drugs or placebos
6. Phase 2 paclitaxel dose optimization stage only: history of prior partial or total gastrectomy
7. Treatment with any systemic anticancer therapy (including hormonal and biologic therapy), radiation, or an experimental agent, or participation in another interventional clinical trial ≤3 weeks prior to first dose of study treatment.
8. Major surgery within 28 days prior to enrollment or randomization, central venous access device placement within 7 days prior to enrollment or randomization, or planned major surgery following initiation of study treatment
9. Any toxicity related to prior cancer therapies that has not resolved to ≤Grade 1, with the following exceptions:
   Anemia
   Alopecia
   Congestive heart failure (CHF), which must have been ≤Grade 1 in severity at the time of occurrence, and must have resolved completely
10. Clinically significant cardiopulmonary disease such as:
   Ventricular arrhythmia requiring therapy
   Symptomatic hypertension or uncontrolled asymptomatic hypertension ≥150/≥90 mmHg despite standard medical management, as determined by the investigator
   Any symptomatic history of CHF, left ventricular systolic dysfunction or decrease in ejection fraction
   Severe dyspnea at rest (CTCAE Grade ≥3) due to complications of advanced malignancy or hypoxia requiring supplementary oxygen therapy, except when therapy is needed for obstructive sleep apnea
11. Known myocardial infarction or unstable angina within 6 months prior to first dose of study treatment
12. Known to be positive for hepatitis B by surface antigen expression. Known to be positive for hepatitis C infection (positive by polymerase chain reaction). Subjects who have been treated for hepatitis C infection are permitted if they have documented sustained virologic response of 12 weeks
13. Presence of known chronic liver disease
14. Phase 2: Known to be positive for human immunodeficiency virus (HIV) Phase 3: Subjects known to be positive for HIV are excluded if they meet any of the following criteria:
   CD4+ T-cell count of <350 cells/uL
   Detectable HIV viral load
   History of an opportunistic infection within the past 12 months
   On stable antiretroviral therapy for <4 weeks
15. Subjects who are pregnant, breastfeeding, or planning to become pregnant from time of informed consent until 7 months following the last dose of study drug
16. Have used a strong cytochrome P450 (CYP) 2C8 inhibitor within 5 half-lives of the inhibitor, or have used a strong CYP2C8 or CYP3A4 inducer within 5 days prior to first dose of study treatment.
17. History of malignancy other than GEC within 2 years prior to screening, with the exception of those with a negligible risk of metastasis or death (e.g., 5-year OS of ≥90%), such as adequately treated carcinoma in situ of the cervix, non-melanoma skin carcinoma, localized prostate cancer, ductal carcinoma in situ, or Stage I uterine cancer.
18. History of deep vein thrombosis, pulmonary embolism, or any other significant thromboembolism during the 3 months prior to enrollment or randomization.
19. Therapeutic anticoagulation with warfarin, low-molecular weight heparin or similar agents. Subjects receiving prophylactic, low-dose anticoagulation therapy are eligible provided that the coagulation parameters defined in the inclusion criteria ([INR≤1.5 and PTT/aPTT≤1.5 ULN] or [PT≤1.5 ULN and PTT/aPTT≤1.5 ULN]) are met.
20. Chronic therapy with nonsteroidal anti-inflammatory agents (NSAIDs; e.g., indomethacin, ibuprofen, naproxen, or similar agents) or other anti-platelet agents (e.g., clopidogrel, ticlopidine, dipyridamole, anagrelide). Aspirin use at doses up to 325 mg/day is permitted.
21. Significant bleeding disorders, vasculitis, or had a significant bleeding episode from the gastrointestinal tract within 3 months prior to study entry.
22. History of any arterial thrombotic event, including myocardial infarction, unstable angina, cerebrovascular accident, or transient ischemic attack, within 6 months prior to enrollment or randomization.
23. History of gastrointestinal perforation and/or fistulae within 6 months prior to enrollment or randomization.
24. Serious non-healing wound or peptic ulcer or bone fracture within 28 days prior to enrollment or randomization
25. History of bowel obstruction, history or presence of inflammatory enteropathy or extensive intestinal resection (hemicolectomy or extensive small intestine resection with chronic diarrhea), Crohn's disease, ulcerative colitis or chronic diarrhea
26. Active or uncontrolled clinically serious infection
27. Known active central nervous system metastases. Irradiated or resected lesions are permitted, provided the lesions are fully treated and inactive, subject is asymptomatic, and no steroids have been administered for at least 30 days

### Example 2: Combination of Tucatinib and Trastuzumab in Colorectal Cancer PDX Models

In this example, the efficacy of tucatinib and trastuzumab was evaluated in PDX models of HER2 positive CRC. Mice were subcutaneously inoculated with CTG-0121, CTG-0784, or CTG-0383 cells, and subsequently treated with tucatinib, trastuzumab, or a combination of the two drugs (n=10 per group). Tucatinib was administered orally at a dose of 50 mg/kg twice per day for 28 days (study days 0-27). Trastuzumab was administered intraperitoneally at a dose of 20 mg/kg once every three days. Nine doses of trastuzumab were administered, starting on study day 0. A vehicle-only group was included as a negative control.

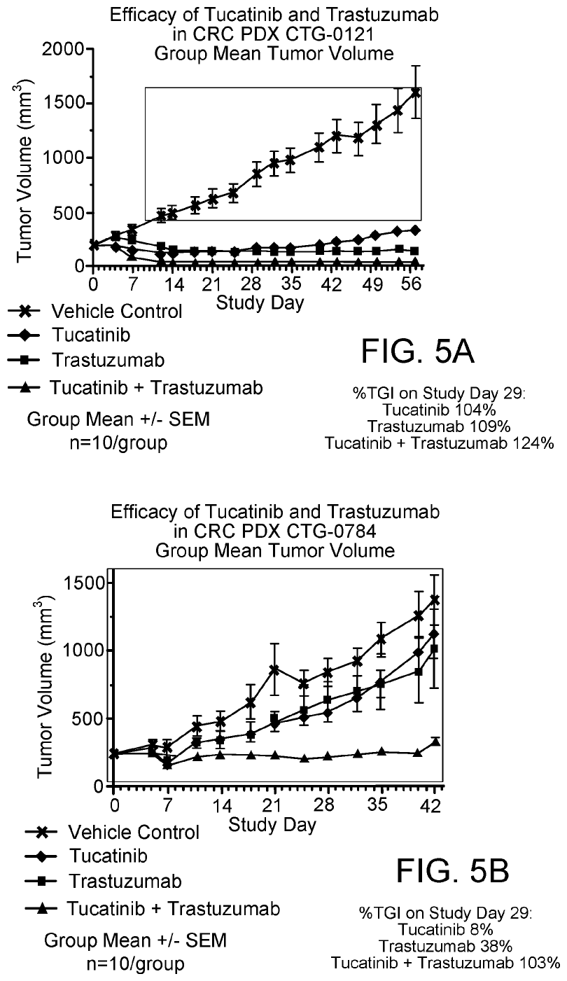

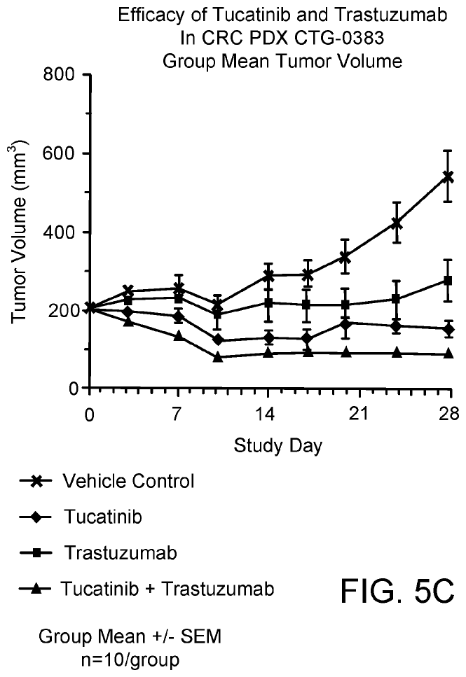

As shown in FIGS. 5A-5C, both tucatinib and trastuzumab inhibited tumor growth in all three CRC PDX models. Furthermore, when a combination of the two drugs was administered, the inhibition of tumor growth was more pronounced than when either drug was used individually. In the CTG-0121 model, tucatinib, trastuzumab, and a combination of the two drugs produced tumor growth inhibition (TGI) indices of 104%, 109%, and 124%, respectively, at study day 29. In the CTG-0784 model, tucatinib, trastuzumab, and a combination of the two drugs produced TGI indices of 50%, 36%, and 103%, respectively, at study day 29. In the CTG-0383 model, tucatinib, trastuzumab, and a combination of the two drugs produced TGI indices of 117%, 80%, and 137%, respectively, at study day 29. Surprisingly, a synergistic effect was observed when a combination of the two drugs was administered in all three models. Of note, the activity of a combination of tucatinib and trastuzumab in each HER2 positive CRC PDX model was comparable to activity observed in a HER2 positive breast cancer model (BT-474).

### Example 3: Combination of Tucatinib and Trastuzumab in Esophageal Cancer PDX Model

In this example, the efficacy of tucatinib and trastuzumab was evaluated in PDX models of HER2 positive esophageal cancer. Mice were subcutaneously inoculated with CTG-0137 or CTG-0138 cells, and subsequently treated with tucatinib, trastuzumab, or a combination of the two drugs (n=10 per group). Tucatinib was administered orally at a dose of 50 mg/kg twice per day for 28 days (study days 0-27). Trastuzumab was administered intraperitoneally at a dose of 20 mg/kg once every three days. Nine doses of trastuzumab were administered, starting on study day 0. A vehicle-only group was included as a negative control.

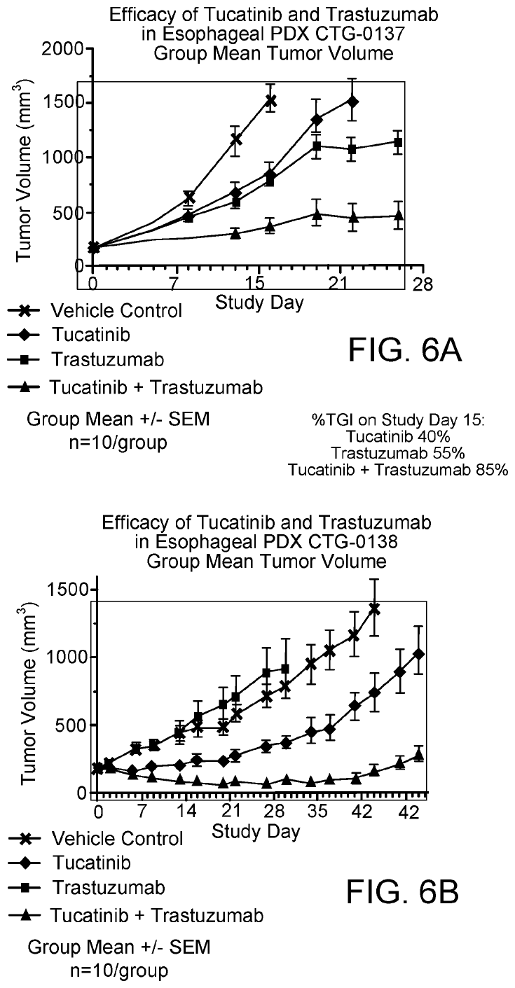

In the CTG-0137 model, both tucatinib and trastuzumab inhibited tumor growth, exhibiting TGI indices at study day 15 of 49% and 55%, respectively (FIG. 6A). Furthermore, a synergistic effect was observed when a combination of the two drugs was administered, producing a TGI index of 85%.

In the CTG-0138 model, tucatinib inhibited tumor growth when administered as a single agent, producing a TGI index of 69% at study day 30 (FIG. 6B). However, a synergistic effect was observed when tucatinib and trastuzumab were administered in combination, producing a TGI index of 120%.

### Example 4: Combination of Tucatinib and Trastuzumab in Gastric Cancer PDX Models

In this example, the efficacy of tucatinib and trastuzumab was evaluated in PDX models of HER2 positive gastric cancer. Mice were subcutaneously inoculated with GXA 3038, GXA 3039, or GXA 3054 cells, and subsequently treated with tucatinib, trastuzumab, or a combination of the two drugs (n=10 per group). Tucatinib was administered orally at a dose of 50 mg/kg twice per day for 28 days (study days 0-27). Trastuzumab was administered intraperitoneally at a dose of 20 mg/kg once every three days. Nine doses of trastuzumab were administered, starting on study day 0. A vehicle-only group was included as a negative control.

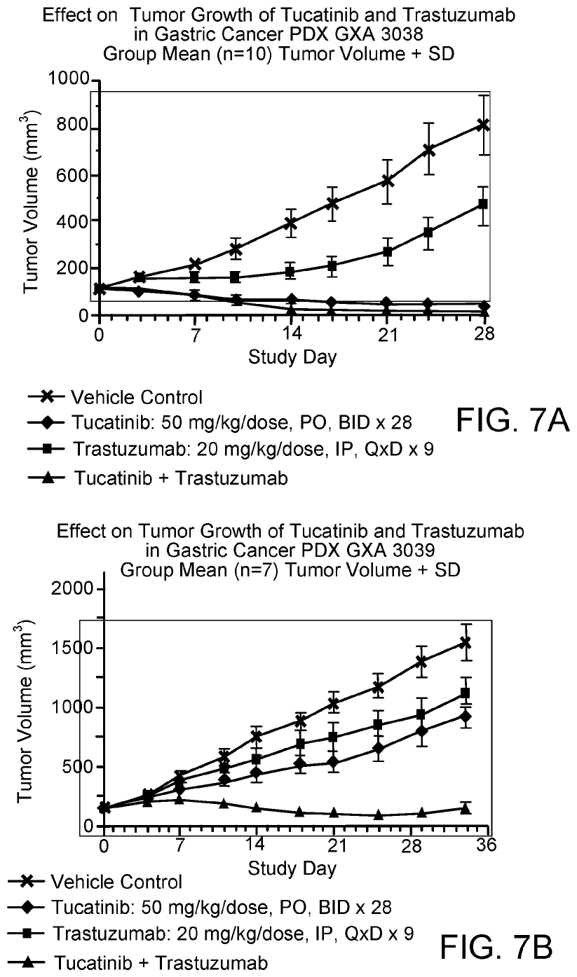

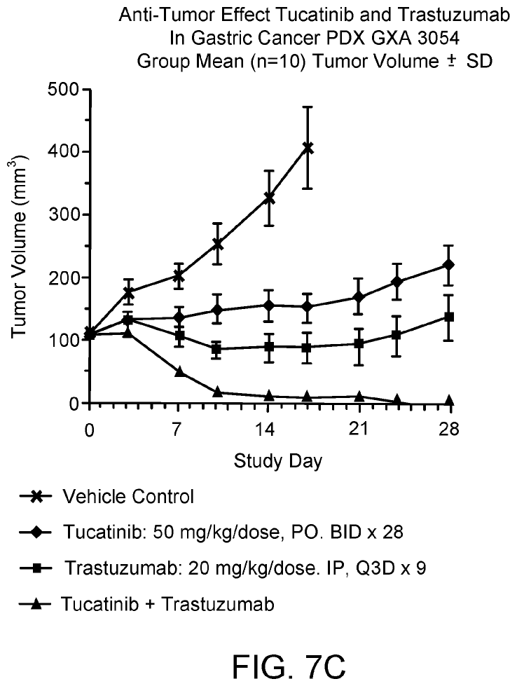

As shown in FIGS. 7A-7C, both tucatinib and trastuzumab inhibited tumor growth in all three gastric cancer PDX models. Furthermore, when a combination of the two drugs was administered, the inhibition of tumor growth was more pronounced than when either drug was used individually. In the GXA-3038 model, tucatinib, trastuzumab, and a combination of the two drugs produced TGI indices of 110%, 50%, and 116%, respectively, at study day 28. In the GXA-3039 model, tucatinib, trastuzumab, and a combination of the two drugs produced TGI indices of 48%, 38%, and 103%, respectively, at study day 29. In the GXA-3054 model, tucatinib, trastuzumab, and a combination of the two drugs produced TGI indices of 65%, 93%, and 136%, respectively, at study day 17. Surprisingly, a synergistic effect was observed when a combination of the two drugs was administered in all three models.

### Example 5: Combination of Tucatinib and Trastuzumab in a Cholangiocarcinoma PDX Model

In this example, the efficacy of tucatinib and trastuzumab was evaluated in a PDX model of HER2 positive cholangiocarcinoma. Mice were subcutaneously inoculated with CTG-0927 cells and subsequently treated with tucatinib, trastuzumab, or a combination of the two drugs (n=10 per group). Tucatinib was administered orally at a dose of 50 mg/kg twice per day for 28 days (study days 0-27). Trastuzumab was administered intraperitoneally at a dose of 20 mg/kg once every three days. Nine doses of trastuzumab were administered, starting on study day 0. A vehicle-only group was included as a negative control.

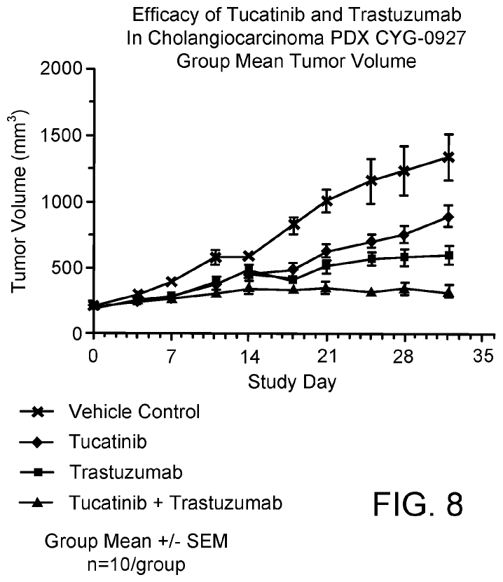

As shown in FIG. 8 both tucatinib and trastuzumab inhibited tumor growth. Furthermore, when a combination of the two drugs was administered, the inhibition of tumor growth was more pronounced than when either drug was used individually. At study day 28, the TGI indices for the tucatinib, trastuzumab, and combination therapy groups were 48%, 63%, and 86%, respectively.

### Example 6: Combination of Tucatinib and Trastuzumab in NSCLC Model

In this example, the efficacy of tucatinib and trastuzumab was evaluated in two different models of HER2 positive NSCLC. For these two studies, Calu-3 and NCI-H2170 cells were used, both of which express high levels of HER2, have gene amplification comparable to that of BT-474 breast cancer cells, and have previously demonstrated good responses to tucatinib in vitro.

Mice were subcutaneously inoculated with Calu-3 or NCI-H2170 cells and subsequently treated with tucatinib, trastuzumab, or a combination of the two drugs (n=10 per group). For the Calu-3 study, tucatinib was administered orally at a dose of 50 mg/kg twice per day for 21 days, beginning on study day 7. Trastuzumab was administered intraperitoneally at a dose of 20 mg/kg once every three days, beginning on study day 7. Seven doses of trastuzumab were administered. A vehicle-only group was included as a negative control. Three individual animals received dose holidays (one in the negative control group and two in the combination therapy group).

For the NCI-H2170 study, tucatinib was administered orally at a dose of 50 mg/kg twice per day for 21 days, beginning on study day 18. Trastuzumab was administered intraperitoneally at a dose of 20 mg/kg twice per week, beginning on study day 18. A vehicle-only group was included as a negative control.

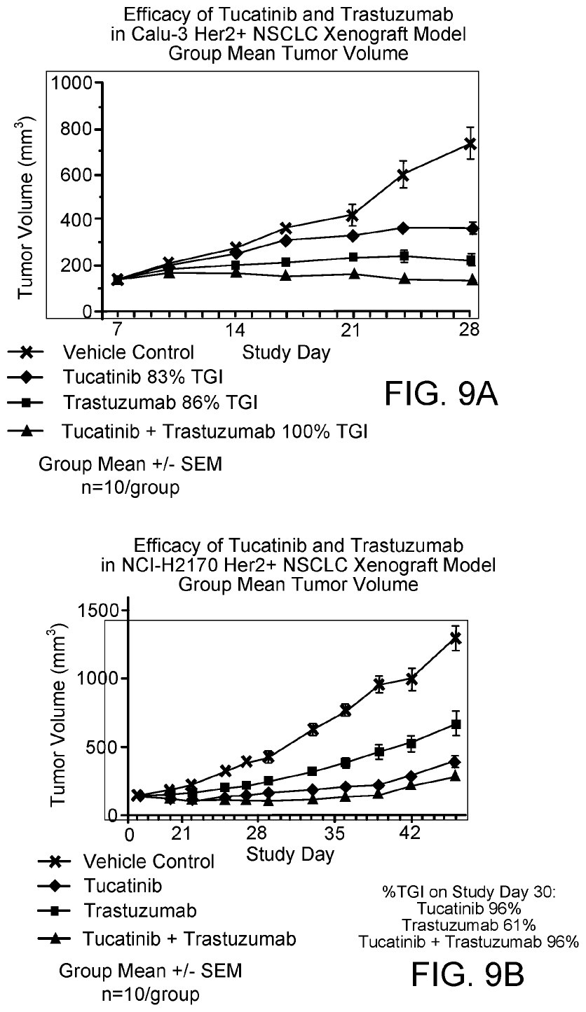

As shown in FIGS. 9A and 9B and Table 15, both tucatinib and trastuzumab inhibited tumor growth in both NSCLC models. Furthermore, when a combination of the two drugs was administered, the inhibition of tumor growth was more pronounced than when either drug was used individually. For the Calu-3 model, tucatinib, trastuzumab, and a combination of the two drugs produced tumor growth inhibition (TGI) indices of 63%, 86%, and 100%, respectively, at study day 28. Surprisingly, a synergistic effect was observed in the combination therapy group. For the NCI-2170 model, tucatinib, trastuzumab, and a combination of the two drugs produced TGI indices of 91%, 61%, and 98%, respectively, at study day 39.

TABLE 15

| | | | | | | | Predicted |
|---|---|---|---|---|---|---|---|
| | | | | Observed TGI (%) | | | % TGI |
| Tumor | Cancer | Type of | | | | Tucatinib + | Tucatinib + |
| name | Type | Xenograft | Vendor | Tucatinib | Trastuzumab | Trastuzumab | Trastuzumab |
| Calu-3 | NSCLC | CDX | BioDuro | 63 | 86 | 100 | 95 |
| NCH- | NSCLC | CDX | In house | 91 | 61 | 98 | 97 |
| H2170 | | | | | | | |
| CTG- | CRC | PDX | Champions | 104 | 109 | 124 | 100 |
| 0121 | | | Oncology | | | | |
| CTG- | CRC | PDX | Champions | 50 | 36 | 103 | 68 |
| 0784 | | | Oncology | | | | |
| CTG- | CRC | PDX | Champions | 117 | 80 | 137 | 103 |
| 0383 | | | Oncology | | | | |
| CTG- | Esophageal | PDX | Champions | 49 | 55 | 85 | 77 |
| 0137 | | | Oncology | | | | |
| CTG- | Esophageal | PDX | Champions | 69 | −34 | 120 | 59 |
| 0138 | | | Oncology | | | | |
| CTG- | Cholangio- | PDX | Champions | 48 | 63 | 86 | 81 |
| 0927 | carcinoma | | Oncology | | | | |
| GXA- | Gastric | PDX | Oncotest | 110 | 50 | 116 | 105 |
| 3038 | carcinoma | | | | | | |
| | (Asian) | | | | | | |
| GXA- | Gastric | PDX | Oncotest | 48 | 38 | 103 | 68 |
| 3039 | carcinoma | | | | | | |
| | (Asian) | | | | | | |
| GXA- | Gastric | PDX | Oncotest | 65 | 93 | 136 | 98 |
| 3054 | carcinoma | | | | | | |
| | (Asian) | | | | | | |

1. A method of treating a HER2 positive cancer in a subject in need thereof, the method comprising administering to the subject a therapeutically effective amount of a combination therapy comprising tucatinib, trastuzumab, a taxane, and a vascular endothelial growth factor receptor 2 (VEGFR-2) antagonist.

2. A method of treating cancer in a subject in need thereof, the method comprising:
(a) identifying the subject as having a HER2 positive cancer; and
(b) administering to the subject a therapeutically effective amount of a combination therapy comprising tucatinib, trastuzumab, a taxane, and a vascular endothelial growth factor receptor 2 (VEGFR-2) antagonist.

3. The method of any one of claim 1 or 2, wherein the trastuzumab is administered to the subject at a dose of about 6 mg/kg.

4. The method of any one of claim 1 or 2, wherein the trastuzumab is administered to the subject at a dose of about 4 mg/kg.

5. The method of any one of claims 1-4, wherein the tucatinib is administered to the subject at a dose of about 150 mg to about 650 mg.

6. The method of any one of claims 1-5, wherein the tucatinib is administered twice daily.

7. The method of any one of claims 1-6, wherein the tucatinib is administered to the subject orally.

8. The method of any one of claims 1-7, wherein the VEGFR-2 antagonist is selected from the group consisting of bevacizumab, ramucirumab, aflibercept, cetuximab, panitumumab, regorafenib, sunitinib, sorafenib, pazopanib, vandetanib, axitinib, cediranib, vatalanib, motesanib, lucatinib, intedanib, semaxanib, apatinib, lenvatinib, carbozantinib, and a combination thereof.

9. The method of any one of claims 1-8, wherein the VEGFR-2 antagonist is a monoclonal antibody selected from the group consisting of bevacizumab, ramucirumab, aflibercept, cetuximab, panitumumab, and combinations thereof.

10. The method of any one of claims 1-9, wherein the VEGFR-2 antagonist is ramucirumab.

11. The method of claim 10, wherein the ramucirumab is administered to the subject at a dose of about 8 mg/kg.

12. The method of any one of claims 1-11 wherein the taxane is selected from the group consisting of paclitaxel, docetaxel, cabazitaxel, larotaxel, BMS-184476, BMS-188797, BMS-275183, milataxel, ortaxel, TL-310, docosahexaenoic acid-paclitaxel (DHA-paclitaxel), nab paclitaxel, EndoTAG+paclitaxel, XRP9881, polymeric-micellar paclitaxel, RPR-109881A, a pharmaceutically acceptable salt or solvate thereof, and a combination thereof.

13. The method of any one of claims 1-12, wherein the taxane is paclitaxel.

14. The method of claim 13, wherein the paclitaxel is administered to the subject at a dose of about 50 mg/m² to about 100 mg/m².

15. The method of any one of claims 12-14, wherein the paclitaxel is administered to the subject at a dose of about 80 mg/m².

16. The method of any one of claims 1-15, wherein the HER2 positive cancer is selected from the group consisting of gastric adenocarcinoma, gastroesophageal junction (GEC) adenocarcinoma, esophageal adenocarcinoma, colorectal carcinoma (CRC), cholangiocarcinoma, gallbladder carcinoma, gastric cancer, lung cancer, biliary cancers, bladder cancer, esophageal cancer, melanoma, ovarian cancer, liver cancer, prostate cancer, pancreatic cancer, small intestine cancer, non-small cell lung cancer, head and neck cancer, uterine cancer, cervical cancer, brain cancer, and breast cancer.

17. The method of any one of claims 1-16, wherein the HER2 positive cancer is gastric adenocarcinoma.

18. The method of any one of claims 1-17, wherein the HER2 positive cancer is gastroesophageal junction (GEC) adenocarcinoma.

19. The method of any one of claims 1-18, wherein the HER2 positive cancer is unresectable or metastatic.

20. The method of any one of claims 1-19, wherein the subject has been previously treated with a HER2-directed antibody.

21. The method of any one of claims 1-20, wherein the subject has not been previously treated with an anti-HER2 and/or an anti-EGFR tyrosine kinase inhibitor.

22. The method of any one of claims 1-21, wherein the subject has not been previously treated with a HER2-directed antibody-drug conjugate.

23. The method of claim 21, wherein the wherein the anti-HER2/EGFR tyrosine kinase inhibitor is selected from the group consisting of tucatinib, lapatinib, neratinib, or afatinib.

24. The method of claim 22, wherein antibody-drug conjugate is selected from the group consisting of ado-trastuzumab (T-DM1) or trastuzumab deruxtecan (DS8201a).

25. The method of any one of claims 1-24, wherein the subject has not been previously treated with an anthracycline.

26. The method of claim 25, wherein the anthracycline is selected from the group consisting of doxorubicin, epirubicin, mitoxantrone, idarubicin, liposomal doxorubicin, and combinations thereof.

27. The method of any one of claims 1-26, wherein the subject was previously treated with at least one anticancer therapy.

28. The method of claim 27, wherein the at least one anticancer therapy is selected from the group consisting of trastuzumab, lapatinib, trastuzumab and a taxane, pertuzumab, and combinations thereof.

29. The method of any one of claim 27 or 28, wherein the subject is refractory to the at least one anticancer therapy.

30. The method of any one of claims 27-29, wherein the subject developed a brain metastasis during the previous treatment with the at least one anticancer therapy.

31. A method for treating a HER2 positive cancer in a subject that has exhibited an adverse event after starting treatment with a combination therapy comprising tucatinib, trastuzumab, a taxane, and a vascular endothelial growth factor receptor 2 (VEGFR-2) antagonist at an initial dosage level, comprising administering to the subject at least one component of the combination therapy at a reduced dosage level.

32. The method of claim 31, wherein the taxane is selected from the group consisting of paclitaxel, docetaxel, cabazitaxel, larotaxel, BMS-184476, BMS-188797, BMS-275183, milataxel, ortaxel, TL-310, docosahexaenoic acid-paclitaxel (DHA-paclitaxel), nab paclitaxel, EndoTAG+paclitaxel, XRP9881, polymeric-micellar paclitaxel, RPR-109881A, a pharmaceutically acceptable salt or solvate thereof, and a combination thereof.

33. The method of any one of claim 31 or 32, wherein the taxane is paclitaxel.

34. The method of claim 33, wherein the paclitaxel is administered to the subject at an initial dose of about 50 mg/m² to about 100 mg/m².

35. The method of any one of claim 33 or 34, wherein the paclitaxel is administered to the subject at an initial dose of about 80 mg/m².

36. The method of any one of claims 33-35, wherein the paclitaxel is administered to the subject at a reduced dose of about 50 mg/m² to about 75 mg/m².

37. The method of any one of claims 33-36 wherein the paclitaxel is administered to the subject at a reduced dose of about 70 mg/m².

38. The method of any one of claims 33-36, wherein the paclitaxel is administered to the subject at a reduced dose of about 60 mg/m².

39. The method of claim 31-38, wherein the tucatinib is administered to the subject at an initial dose of about 150 mg to about 650 mg.

40. The method of claim 31-39, wherein the tucatinib is administered to the subject at an initial dose of about 300 mg.

41. The method of any one of claims 31-40, wherein the tucatinib is administered to the subject at a reduced dose of about 125 mg to about 275 mg.

42. The method of any one of claims 31-41, wherein the VEGFR-2 antagonist is selected from the group consisting of bevacizumab, ramucirumab, aflibercept, cetuximab, panitumumab, regorafenib, sunitinib, sorafenib, pazopanib, vandetanib, axitinib, cediranib, vatalanib, motesanib, lucatinib, intedanib, semaxanib, apatinib, lenvatinib, carbozantinib, and a combination thereof.

43. The method of any one of claims 31-42, wherein the VEGFR-2 antagonist is ramucirumab.

44. The method of any one of claim 42 or 43, wherein the ramucirumab is administered to the subject at an initial dose of about 8 mg/kg.

45. The method of any one of claims 42-44, wherein the ramucirumab is administered to the subject at a reduced dose of about 6 mg/kg.

46. The method of any one of claims 42-44, wherein the ramucirumab is administered to the subject at a reduced dose of about 5 mg/kg.

47. A method of treating a HER2 positive cancer in a subject in need thereof, the method comprising:
(a) administering to the subject a therapeutically effective amount of a combination therapy comprising tucatinib, trastuzumab, a taxane, and a vascular endothelial growth factor receptor 2 (VEGFR-2) antagonist; and
(b) administering an effective amount of an anti-diarrheal agent.

48. A method of reducing the severity or incidents of diarrhea, or preventing diarrhea in a subject having a HER2 positive cancer and being treated with an effective amount of a combination therapy comprising tucatinib, trastuzumab, a taxane, and a vascular endothelial growth factor receptor 2 (VEGFR-2) antagonist, the method comprising administering an effective amount of an anti-diarrheal agent prophylactically.

49. A method of reducing the likelihood of a subject developing diarrhea, wherein the subject has a HER2 positive cancer and is being treated with an effective amount of a combination therapy comprising tucatinib, trastuzumab, a taxane, and a vascular endothelial growth factor receptor 2 (VEGFR-2) antagonist the method comprising administering an effective amount of an anti-diarrheal agent prophylactically.

50. The method of any one of claims 47-49, wherein the combination therapy and the anti-diarrheal agent are administered concurrently.

51. The method of any one of claims 47-49, wherein the anti-diarrheal agent is administered prior to administration of the combination therapy.

52. The method of any one of claims 47-51, wherein the subject is exhibiting symptoms of diarrhea.

53. The method of any one of claims 47-51, wherein the subject is not exhibiting symptoms of diarrhea.


SEQUENCE LISTING (4 sequences)

SEQ ID NO: 1 - protein, 449 aa, Artificial
  trastuzumab heavy chain
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 2 - protein, 214 aa, Artificial
  trastuzumab light chain
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 108 aa, Artificial
  trastuzumab light chain variable domain
DIQMTQSPSSLSASVGDRVTITCRASQDVNTAVAWYQQKPGKAPKLLIYSASFLESGVPSRFSGSRSGTDFTLTISSLQPEDFATYYCQQHYTTPPTFGQTKVEIKRT

SEQ ID NO: 4 - protein, 120 aa, Artificial
  trastuzumab heavy chain variable domain
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDVWGQGTLVTVSS